(19) United States
(12) Patent Application Publication
(10) Pub. No.: US 2023/0183362 A1
(43) Pub. Date: Jun. 15, 2023

(54) METHODS FOR TREATING PRURIGO NODULARIS BY ADMINISTERING AN IL-4R ANTAGONIST

(71) Applicants: SANOFI BIOTECHNOLOGY, Paris (FR); REGENERON PHARMACEUTICALS, INC., Tarrytown, NY (US)

(72) Inventors: Elizabeth Laws, Paris (FR); John O'Malley, Bridgewater, NJ (US); Heribert Staudinger, Bridgewater, NJ (US); Naimish Patel, Bridgewater, NJ (US); Ashish Bansal, Tarrytown, NY (US)

(21) Appl. No.: 17/969,033

(22) Filed: Oct. 19, 2022

Related U.S. Application Data
(60) Provisional application No. 63/300,492, filed on Jan. 18, 2022.
(60) Provisional application No. 63/257,876, filed on Oct. 20, 2021.

(30) Foreign Application Priority Data
Mar. 4, 2022 (EP) 22315048.3

(51) Int. Cl. C07K 16/28; A61P 17/04; A61P 17/02

(52) U.S. Cl. CPC C07K 16/2866; A61P 17/04; A61P 17/02; A61K 2039/505

(57) ABSTRACT

Methods for treating or preventing prurigo nodularis (PN) in a subject are provided. Methods comprising administering to a subject in need thereof a therapeutic composition comprising an interleukin-4 receptor (IL-4R) antagonist, such as an anti-IL-4R antibody or antigen-binding fragment thereof, are provided.

### RELATED APPLICATIONS

This application claims the benefit of U.S. Provisional Application Nos. 63/257,876 filed Oct. 20, 2021, and 63/300,492 filed Jan. 18, 2022, and EP Application No. EP 22315048.3 filed Mar. 4, 2022, each of which is incorporated by reference in its entirety.

### SEQUENCE LISTING

The content of the electronically submitted Sequence Listing in XML format (Name: 732138_SA9-314_ST26.xml; Size: 285,145 bytes; and Date of Creation: Feb. 24, 2023) is incorporated herein by reference in its entirety.

### FIELD OF THE INVENTION

The disclosure relates to the treatment and/or prevention of prurigo nodularis (PN) in a subject in need thereof. The disclosure relates to the administration of an interleukin-4 receptor (IL-4R) antagonist to treat or prevent PN in a subject in need thereof.

### BACKGROUND

Prurigo nodularis (PN) is a skin disease characterized by multiple, intensely itchy skin eruptions in symmetrically distributed areas of the extremities. (Zeidler C, et al. Chronic prurigo of nodular type: A Review. Acta Dermatol Venereol 2018; 98(2):173-9.) The main symptom is prolonged, repetitive and uncontrollable rubbing, scratching and uncontrollable itching which leads to hyperkeratotic eroding papules and nodules on the skin. A broadly accepted definition for chronic prurigo has been published by the European Academy of Dermatology and Venereology (EADV). (Pereira M P, et al. European academy of dermatology and venereology European prurigo project: expert consensus on the definition, classification and terminology of chronic prurigo. J Eur Acad Dermatol Venereol. 2018; 32(7):1059-65.) The convened experts agreed that chronic prurigo should be used as an umbrella term for the range of clinical manifestations (e.g., papular, nodular, plaque or umbilicated types). Prurigo nodularis is considered a distinct disease defined by the presence of chronic pruritus for ≥6 weeks, history and/or signs of repeated scratching and multiple localized/generalized pruriginous skin lesions (whitish, hyperpigmented, or pink papules, nodules and/or plaques.)

It is difficult to treat and entails a high disease burden. Approximately 50% of patients have either past or current history of atopic dermatitis (AD) or other atopic disorders. (Iking A, et al., Stander S. Prurigo as a symptom of atopic and non-atopic diseases: aetiological survey in a consecutive cohort of 108 patients. J Eur Acad Dermatol Venereol 2013; 27(5):550-7.)

Due to the central manifestation of itch, PN carries a significant burden of disease. (Pereira M P, et al. European academy of dermatology and venereology European prurigo project: expert consensus on the definition, classification and terminology of chronic prurigo. J Eur Acad Dermatol Venereol 2018; 32(7):1059-65.) The effect on quality of life due to PN has been reported to be higher than other common skin disorders like AD and psoriasis. (Steinke S, et al. Humanistic burden of chronic pruritus in patients with inflammatory dermatoses: Results of the European Academy of Dermatology and Venereology Network on Assessment of Severity and Burden of Pruritus (PruNet) cross-sectional trial. J Am Acad Dermatol 2018; 79(3):457-63.) Patients report chronic sleep loss due to constant itching; constant burning, stinging, and pain at affected area; and chronic depression, anxiety, anger, disgust, and shame; and hence overall experience a great impact on their quality of life. According to a 5-year cross-sectional study on 909 adult PN patients in the Johns Hopkins hospital system (Boozalis E, et al. Ethnic differences and comorbidities of 909 prurigo nodularis patients J Am Acad Dermatol 2018; 79(4):714-9), PN is a key contributing factor to mood disorders such as anxiety and depression.

Data on the epidemiology of PN are limited. Most studies show a predominance of older patients with a median age of more than 50 years. (Iking A, et al. Prurigo as a symptom of atopic and non-atopic diseases: aetiological survey in a consecutive cohort of 108 patients. J Eur Acad Dermatol Venereol. 2013; 27(5):550-7.) Prurigo nodularis is only occasionally observed in younger patients, in whom it is often associated with atopic conditions. (Tanaka M, et al. Prurigo nodularis consists of two distinct forms: early-onset atopic and late-onset non-atopic. Dermatology. 1995; 190(4):26976.)

There are no United States (US) Food and Drug Administration (FDA) or European Medicines Agency (EMA) approved targeted therapies indicated for the treatment of PN, whether atopic or non-atopic forms. Accordingly, a need exists for novel therapies to treat PN.

### BRIEF SUMMARY OF THE INVENTION

In one aspect, a method for treating a subject having prurigo nodularis comprising administering to the subject an initial dose of about 600 mg of an antibody or an antigen-binding fragment thereof that specifically binds to interleukin-4 receptor (IL-4R) comprising three heavy chain complementarity determining region (HCDR) sequences comprising SEQ ID NOs: 3, 4, and 5, and three light chain complementarity determining region (LCDR) sequences comprising SEQ ID NOs: 6, 7, and 8, and one or more secondary doses of about 300 mg of the antibody or the antigen-binding fragment thereof, is provided.

In certain exemplary embodiments, the secondary doses are administered every other week (q2w).

In certain exemplary embodiments, the subject was previously ineffectively treated with medium-to-superpotent topical corticosteroids.

In certain exemplary embodiments, the subject has a baseline WI-NRS score that is equal to or greater than 7.

In certain exemplary embodiments, the subject has a minimum of 20 PN nodules in total on both legs, and/or both arms and/or trunk at baseline.

In certain exemplary embodiments, the subject has a baseline IGA PN score of greater than or equal to 3.

In certain exemplary embodiments, the subject has PN that is not adequately controlled with topical therapies or when those therapies are not advisable.

In certain exemplary embodiments, the subject is a candidate for systemic therapy.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2.

In certain exemplary embodiments, the antibody is dupilumab.

In certain exemplary embodiments, the antibody or antigen-binding fragment thereof is administered using an autoinjector, a needle and syringe, or a pen.

In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, the subject is an adult.

In another aspect, a method for the treatment of prurigo nodularis that reduces or eliminates a prurigo nodularis patient's dependence on low to medium potency topical corticosteroids and/or topical calcineurin inhibitors comprising (a) selecting a patient with prurigo nodularis that is uncontrolled with a background therapy comprising low to medium potency topical corticosteroids and/or topical calcineurin inhibitors; (b) administering to the patient a defined dose of an antibody or antigen-binding fragment thereof that specifically binds to an interleukin-4 receptor (IL-4R) at a defined frequency for an initial treatment period while maintaining the patient's background therapy for the initial treatment period; and (c) gradually reducing or eliminating the dosage of low to medium potency topical corticosteroids and/or topical calcineurin inhibitors administered to the patient over the course of a subsequent treatment period while continuing to administer the antibody or antigen-binding fragment thereof at the defined frequency and dose used during the initial treatment period, wherein the antibody or antigen-binding fragment thereof comprises three heavy chain complementarity determining region (HCDR) sequences comprising SEQ ID NOs: 3, 4, and 5, and three light chain complementarity determining region (LCDR) sequences comprising SEQ ID NOs: 6, 7, and 8, is provided.

In certain exemplary embodiments, the antibody or antigen-binding fragment thereof is administered to the subject as an initial dose followed by one or more secondary doses.

In certain exemplary embodiments, the initial dose is about 300 mg and each secondary dose is about 300 mg.

In certain exemplary embodiments, the initial dose is about 600 mg and each secondary dose is about 300 mg.

In certain exemplary embodiments, the secondary doses are administered every other week (q2w).

In certain exemplary embodiments, the subject was previously ineffectively treated with medium-to-superpotent topical corticosteroids.

In certain exemplary embodiments, the subject has a baseline WI-NRS score that is equal to or greater than 7.

In certain exemplary embodiments, the subject has a minimum of 20 PN nodules in total on both legs, and/or both arms and/or trunk at baseline.

In certain exemplary embodiments, the subject has a baseline IGA PN score of greater than or equal to 3.

In certain exemplary embodiments, the subject has PN that is not adequately controlled with topical therapies or when those therapies are not advisable.

In certain exemplary embodiments, the subject is a candidate for systemic therapy.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2.

In certain exemplary embodiments, the antibody is dupilumab.

In certain exemplary embodiments, the antibody or antigen-binding fragment thereof is administered using an autoinjector, a needle and syringe, or a pen.

In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, the subject is an adult.

In another aspect, a method for treating a subject having prurigo nodularis comprising administering to the subject an initial dose of an antibody or an antigen-binding fragment thereof that specifically binds to interleukin-4 receptor (IL-4R) comprising three heavy chain complementarity determining region (HCDR) sequences comprising SEQ ID NOs: 3, 4, and 5, and three light chain complementarity determining region (LCDR) sequences comprising SEQ ID NOs: 6, 7, and 8, and one or more secondary doses of the antibody or the antigen-binding fragment thereof, wherein the treatment with the antibody or antigen-binding fragment thereof results in a decrease in the need for treatment of the subject with superpotent topical corticosteroid rescue medication, is provided.

In certain exemplary embodiments, the initial dose is about 300 mg and each secondary dose is about 300 mg.

In certain exemplary embodiments, the initial dose is about 600 mg and each secondary dose is about 300 mg.

In certain exemplary embodiments, the secondary doses are administered every other week (q2w).

In certain exemplary embodiments, the subject was previously ineffectively treated with medium-to-superpotent topical corticosteroids.

In certain exemplary embodiments, the subject has a baseline WI-NRS score that is equal to or greater than 7.

In certain exemplary embodiments, the subject has a minimum of 20 PN nodules in total on both legs, and/or both arms and/or trunk at baseline.

In certain exemplary embodiments, the subject has a baseline IGA PN score of greater than or equal to 3.

In certain exemplary embodiments, the subject has PN that is not adequately controlled with topical therapies or when those therapies are not advisable.

In certain exemplary embodiments, the subject is a candidate for systemic therapy.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2.

In certain exemplary embodiments, the antibody is dupilumab.

In certain exemplary embodiments, the antibody or antigen-binding fragment thereof is administered using an autoinjector, a needle and syringe, or a pen.

In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, the subject is an adult.

In another aspect, a method for treating pruritus associated with prurigo nodularis in a subject comprising administering to the subject an initial dose of an antibody or an antigen-binding fragment thereof that specifically binds to interleukin-4 receptor (IL-4R) comprising three heavy chain complementarity determining region (HCDR) sequences comprising SEQ ID NOs: 3, 4, and 5, and three light chain complementarity determining region (LCDR) sequences comprising SEQ ID NOs: 6, 7, and 8, and one or more secondary doses of the antibody or the antigen-binding fragment thereof, wherein the treatment with the antibody or antigen-binding fragment thereof results in a decrease in the need for treatment of the subject with superpotent topical corticosteroid rescue medication, is provided.

In certain exemplary embodiments, the initial dose is about 300 mg and each secondary dose is about 300 mg.

In certain exemplary embodiments, the initial dose is about 600 mg and each secondary dose is about 300 mg.

In certain exemplary embodiments, the secondary doses are administered every other week (q2w).

In certain exemplary embodiments, the pruritus is refractory to topical therapy.

In certain exemplary embodiments, the subject was previously ineffectively treated with medium-to-superpotent topical corticosteroids.

In certain exemplary embodiments, the subject has a baseline WI-NRS score that is equal to or greater than 7.

In certain exemplary embodiments, the subject has a minimum of 20 PN nodules in total on both legs, and/or both arms and/or trunk at baseline.

In certain exemplary embodiments, the subject has a baseline IGA PN score of greater than or equal to 3.

In certain exemplary embodiments, the subject has PN that is not adequately controlled with topical therapies or when those therapies are not advisable.

In certain exemplary embodiments, the subject is a candidate for systemic therapy.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2.

In certain exemplary embodiments, the antibody is dupilumab.

In certain exemplary embodiments, the antibody or antigen-binding fragment thereof is administered using an autoinjector, a needle and syringe, or a pen.

In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, the subject is an adult.

In another aspect, a method for treating a subject having prurigo nodularis comprising administering to the subject an initial dose of an antibody or an antigen-binding fragment thereof that specifically binds to interleukin-4 receptor (IL-4R) comprising three heavy chain complementarity determining region (HCDR) sequences comprising SEQ ID NOs: 3, 4, and 5, and three light chain complementarity determining region (LCDR) sequences comprising SEQ ID NOs: 6, 7, and 8, and one or more secondary doses of the antibody or the antigen-binding fragment thereof, wherein the treatment with the antibody or antigen-binding fragment thereof results in a decrease in the need for treatment of the subject with systemic immunosuppressants, is provided.

In certain exemplary embodiments, the initial dose is about 300 mg and each secondary dose is about 300 mg.

In certain exemplary embodiments, the initial dose is about 600 mg and each secondary dose is about 300 mg.

In certain exemplary embodiments, the secondary doses are administered every other week (q2w).

In certain exemplary embodiments, the subject was previously ineffectively treated with medium-to-superpotent topical corticosteroids.

In certain exemplary embodiments, the subject has a baseline WI-NRS score that is equal to or greater than 7.

In certain exemplary embodiments, the subject has a minimum of 20 PN nodules in total on both legs, and/or both arms and/or trunk at baseline.

In certain exemplary embodiments, the subject has a baseline IGA PN score of greater than or equal to 3.

In certain exemplary embodiments, the subject has PN that is not adequately controlled with topical therapies or when those therapies are not advisable.

In certain exemplary embodiments, the subject is a candidate for systemic therapy.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2.

In certain exemplary embodiments, the antibody is dupilumab.

In certain exemplary embodiments, the antibody or antigen-binding fragment thereof is administered using an autoinjector, a needle and syringe, or a pen.

In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, the subject is an adult.

In another aspect, a method for treating a subject having prurigo nodularis comprising administering to the subject an initial dose of an antibody or an antigen-binding fragment thereof that specifically binds to interleukin-4 receptor (IL-4R) comprising three heavy chain complementarity determining region (HCDR) sequences comprising SEQ ID NOs: 3, 4, and 5, and three light chain complementarity determining region (LCDR) sequences comprising SEQ ID NOs: 6, 7, and 8, and one or more secondary doses of the antibody or the antigen-binding fragment thereof, and wherein the treatment results in the subject having a decrease in worst itch numeric rating scale (WI-NRS) score, is provided.

In certain exemplary embodiments, the decrease in WI-NRS score is selected from the group consisting of 4, 5, 6, 7, 8, 9, and 10.

In certain exemplary embodiments, the decrease in WI-NRS score occurs with 12 weeks of treatment.

In certain exemplary embodiments, the decrease in WI-NRS score occurs with 24 weeks of treatment.

In certain exemplary embodiments, the initial dose is about 300 mg and each secondary dose is about 300 mg.

In certain exemplary embodiments, the initial dose is about 600 mg and each secondary dose is about 300 mg.

In certain exemplary embodiments, the secondary doses are administered every other week (q2w).

In certain exemplary embodiments, the subject was previously ineffectively treated with medium-to-superpotent topical corticosteroids.

In certain exemplary embodiments, the subject has a baseline WI-NRS score that is equal to or greater than 7.

In certain exemplary embodiments, the subject has a minimum of 20 PN nodules in total on both legs, and/or both arms and/or trunk at baseline.

In certain exemplary embodiments, the subject has a baseline IGA PN score of greater than or equal to 3.

In certain exemplary embodiments, the subject has PN that is not adequately controlled with topical therapies or when those therapies are not advisable.

In certain exemplary embodiments, the subject is a candidate for systemic therapy.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2.

In certain exemplary embodiments, the antibody is dupilumab.

In certain exemplary embodiments, the antibody or antigen-binding fragment thereof is administered using an autoinjector, a needle and syringe, or a pen.

In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, the subject is an adult.

In another aspect, a method for treating a subject having prurigo nodularis comprising administering to the subject an initial dose of an antibody or an antigen-binding fragment thereof that specifically binds to interleukin-4 receptor (IL-4R) comprising three heavy chain complementarity determining region (HCDR) sequences comprising SEQ ID NOs: 3, 4, and 5, and three light chain complementarity determining region (LCDR) sequences comprising SEQ ID NOs: 6, 7, and 8, and one or more secondary doses of the antibody or the antigen-binding fragment thereof, wherein the treatment results in the subject having a decrease in investigator's global assessment for prurigo nodularis (IGA PN) score, is provided.

In certain exemplary embodiments, the decrease in IGA PN score is selected from the group consisting of 5, 4, 3, 2, and 1.

In certain exemplary embodiments, the subject achieves an IGA PN score of 0 or 1.

In certain exemplary embodiments, the decrease in IGA PN score occurs with 12 weeks of treatment.

In certain exemplary embodiments, the decrease in IGA PN score occurs with 24 weeks of treatment.

In certain exemplary embodiments, the initial dose is about 300 mg and each secondary dose is about 300 mg.

In certain exemplary embodiments, the initial dose is about 600 mg and each secondary dose is about 300 mg.

In certain exemplary embodiments, the secondary doses are administered every other week (q2w).

In certain exemplary embodiments, the subject was previously ineffectively treated with medium-to-superpotent topical corticosteroids.

In certain exemplary embodiments, the subject has a baseline WI-NRS score that is equal to or greater than 7.

In certain exemplary embodiments, the subject has a minimum of 20 PN nodules in total on both legs, and/or both arms and/or trunk at baseline.

In certain exemplary embodiments, the subject has a baseline IGA PN score of greater than or equal to 3.

In certain exemplary embodiments, the subject has PN that is not adequately controlled with topical therapies or when those therapies are not advisable.

In certain exemplary embodiments, the subject is a candidate for systemic therapy.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2.

In certain exemplary embodiments, the antibody is dupilumab.

In certain exemplary embodiments, the antibody or antigen-binding fragment thereof is administered using an autoinjector, a needle and syringe, or a pen.

In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, the subject is an adult.

In another aspect, a method for treating a subject having prurigo nodularis comprising administering to the subject an initial dose of an antibody or an antigen-binding fragment thereof that specifically binds to interleukin-4 receptor (IL-4R) comprising three heavy chain complementarity determining region (HCDR) sequences comprising SEQ ID NOs: 3, 4, and 5, and three light chain complementarity determining region (LCDR) sequences comprising SEQ ID NOs: 6, 7, and 8, and one or more secondary doses of the antibody or the antigen-binding fragment thereof, wherein the subject has co-morbid mild atopic dermatitis, is provided.

In certain exemplary embodiments, the initial dose is about 300 mg and each secondary dose is about 300 mg.

In certain exemplary embodiments, the initial dose is about 600 mg and each secondary dose is about 300 mg.

In certain exemplary embodiments, the secondary doses are administered every other week (q2w).

In certain exemplary embodiments, the subject was previously ineffectively treated with medium-to-superpotent topical corticosteroids.

In certain exemplary embodiments, the subject has a baseline WI-NRS score that is equal to or greater than 7.

In certain exemplary embodiments, the subject has a minimum of 20 PN nodules in total on both legs, and/or both arms and/or trunk at baseline.

In certain exemplary embodiments, the subject has a baseline IGA PN score of greater than or equal to 3.

In certain exemplary embodiments, the subject has PN that is not adequately controlled with topical therapies or when those therapies are not advisable.

In certain exemplary embodiments, the subject is a candidate for systemic therapy.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2.

In certain exemplary embodiments, the antibody is dupilumab.

In certain exemplary embodiments, the antibody or antigen-binding fragment thereof is administered using an autoinjector, a needle and syringe, or a pen.

In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, the subject is an adult.

In another aspect, a method for treating a subject having prurigo nodularis comprising selecting a subject having prurigo nodularis, and administering to the subject an initial dose of about 600 mg of an antibody or an antigen-binding fragment thereof that specifically binds to interleukin-4 receptor (IL-4R) comprising three heavy chain complementarity determining region (HCDR) sequences comprising SEQ ID NOs: 3, 4, and 5, and three light chain complementarity determining region (LCDR) sequences comprising SEQ ID NOs: 6, 7, and 8, and one or more secondary doses of about 300 mg of the antibody or the antigen-binding fragment thereof is provided.

In another aspect, an antibody or an antigen-binding fragment thereof that specifically binds to interleukin-4 receptor (IL-4R) comprising three heavy chain complementarity determining region (HCDR) sequences comprising SEQ ID NOs: 3, 4, and 5, and three light chain complementarity determining region (LCDR) sequences comprising SEQ ID NOs: 6, 7, and 8, and comprising an initial dose of about 600 mg of the antibody or the antigen-binding fragment thereof, and one or more secondary doses of about 300 mg of the antibody or the antigen-binding fragment thereof, for use in treating prurigo nodularis is provided.

In another aspect, a use of an antibody or an antigen-binding fragment thereof that specifically binds to interleukin-4 receptor (IL-4R) comprising three heavy chain complementarity determining region (HCDR) sequences comprising SEQ ID NOs: 3, 4, and 5, and three light chain complementarity determining region (LCDR) sequences comprising SEQ ID NOs: 6, 7, and 8, for the manufacture of a medicament for the treatment of prurigo nodularis, wherein the use comprises administering an initial dose of about 600 mg of the antibody or the antigen-binding fragment thereof, and one or more secondary doses of about 300 mg of the antibody or the antigen-binding fragment thereof is provided.

### BRIEF DESCRIPTION OF THE FIGURES

The foregoing and other features and advantages of the disclosure will be more fully understood from the following detailed description of illustrative embodiments taken in conjunction with the accompanying drawings. The file of this patent contains at least one drawing/photograph executed in color. Copies of this patent with color drawing(s)/photograph(s) will be provided by the Office upon request and payment of the necessary fee.

FIG. 1 schematically depicts the overview of the study design of Example 1. The study was a multi-center, 24-week treatment, parallel, double-blind, randomized, placebo-controlled study to evaluate the use of dupilumab in patients with PN inadequately controlled on topical prescription therapies or when those therapies are not advisable. Participants received either dupilumab in a 600 mg loading dose followed by 300 mg every other week (q2w) or matched placebo.

FIG. 2A-FIG. 2D depict a table of the schedule of activities for the two randomized, placebo-controlled studies of dupilumab in patients with PN inadequately controlled on topical prescription therapies or when those therapies are not advisable (Example 1).

FIG. 3 depicts the questionnaire used for determining worst itch numeric rating scale (WI-NRS).

FIG. 4 depicts the questionnaire used for determining investigator's global assessment of prurigo nodularis (IGA PN).

FIG. 5 depicts the questionnaire used for determining prurigo activity score (PAS).

FIG. 6 schematically depicts the two Phase 3 studies of similar design and population described in Example 1. Both studies evaluated the use of dupilumab in patients with PN inadequately controlled on topical prescription therapies or in patients with for which topical prescription therapies were not advisable.

FIG. 7A-B depict tables of the statistical testing hierarchy for the PRIME and PRIME2 studies of Example 1. As shown in FIG. 7A, for PRIME, the primary and all multiplicity adjusted secondary endpoints were met with statistical significance including WI-NRS≥4, IGA PN-S score of 0 or 1, WI-NRS≥4 and IGA PN-S score of 0 or 1, WI-NRS (itch) percent change from baseline, DLQI change from baseline, skin pain-NRS change from baseline, and HADS change from baseline (all at 24 weeks). As shown in FIG. 7B, for PRIME2, primary, key secondary, and other multiplicity-controlled endpoints were met with statistical significance including WI-NRS≥4 at 12 and 24 weeks, IGA PN-S score of 0 or 1 at 12 and 24 weeks, WI-NRS≥4 and IGA PN-S score of 0 or 1 at 24 weeks, WI-NRS % mean change from baseline at 24 weeks, DLQI at 24 weeks, and skin pain-NRS at 24 weeks.

FIG. 8A-D graphically depict the proportion of patients with WI-NRS≥4 for both placebo and dupilumab treatment groups. For PRIME, as shown in FIG. 8A, the proportion of participants who reached ≥4-point reduction of WI-NRS score (0-10) at week 24 with dupilumab was 45 (60.0%) and with placebo was 14 (18.4%), p<0.0001. For PRIME2, as shown in FIG. 8B, the proportion of participants who reached ≥4-point reduction of WI-NRS (0-10) at week 12 with dupilumab was 29 (37.2%) and with placebo was 18 (22.0%), p=0.0216. The proportion of participants who reached ≥4-point reduction of WI-NRS (0-10) at week 24 with dupilumab was 57.7% and with placebo was 19.5%, (p<0.0001). FIG. 8C-D graphically depict the proportion of participants with a WI-NRS improvement from baseline ≥4 over time until week 36 in the PRIME study (FIG. 8C) and PRIME2 study (FIG. 8D).

FIG. 9A-D graphically depict the proportion of participants who reached an Investigator's Global Assessment PN-Stage (IGA PN-S) score of 0 or 1 for the dupilumab and placebo treatment groups. For PRIME, as shown in FIG. 9A, the proportion of participants who reached an IGA PN-S score of 0 or 1 at week 24 with dupilumab was 48.0% and with placebo was 18.4%, (p=0.0004). For PRIME2, as shown in FIG. 9B, the proportion of participants who reached an IGA PN-S score of 0 or 1 at week 24 with dupilumab was 44.9% and with placebo was 15.9%, (p<0.0001). For week 12, the proportion was 25.6% with dupilumab and 12.2% with placebo, (p=0.0194). FIG. 9C-D graphically depict the proportion of participants with an IGA PN-S score of 0 or 1 score from baseline over time until week 36 in the PRIME study (FIG. 9C) and PRIME 2 study (FIG. 9D).

FIG. 10A-D graphically depict the proportion of participants with concomitant improvement (reduction) in WI-NRS by ≥4 from baseline and an IGA PN-S score of 0 or 1 for the dupilumab and placebo treatment groups. For PRIME, as shown in FIG. 10A, the proportion of participants with concomitant improvement (reduction) in WI-NRS by ≥4 from baseline to week 24 and an IGA PN-S score of 0 or 1 at week 24 with dupilumab was 38.7% and with placebo was 9.2%, (p<0.0001). For PRIME2, as shown in FIG. 10B, the proportion of participants with concomitant improvement (reduction) in WI-NRS by ≥4 from baseline to week 24 and an IGA PN-S score of 0 or 1 at week 24 with dupilumab was 32.1% and with placebo was 8.5%, (p=0.0001). FIG. 10C-D graphically depict the proportion of participants with both an improvement (reduction) in WI-NRS by ≥4 from baseline and an IGA PN-S score of 0 or 1 over time up to week 36 in the PRIME study (FIG. 10C) and PRIME2 study (FIG. 10D).

FIG. 11A-D graphically depict WI-NRS least squares (LS) mean % A from baseline for the dupilumab and placebo treatment groups. For PRIME, as shown in FIG. 11A, at week 24, the LS mean % change from baseline was −48.89 for the dupilumab treatment group and −22.22 for the placebo treatment group (p<0.0001). For PRIME2, as shown in FIG. 11B, at week 24, the LS mean % change from baseline was −59.34 for the dupilumab treatment group and −36.18 for the placebo treatment group (p<0.0001). FIG. 11C-D graphically depict the mean percent change from baseline in WI-NRS over time up to week 36 in the PRIME study (FIG. 11C) and PRIME2 study (FIG. 11D).

FIG. 12A-B graphically depict time to first use of rescue and/or prohibited medications or procedures. In both PRIME (FIG. 12A) and PRIME2 (FIG. 12B), dupilumab treatment as compared to placebo reduced the time to first use of rescue and/or prohibited medications or procedures.

FIG. 13A-B depicts the skin of a patient in the PRIME study at baseline (FIG. 13A) and at week 24 (FIG. 13B) after starting treatment with 300 mg dupilumab administered q2w. At baseline the patient had an IGA PN-S score of 4 and an average WI-NRS score of 9.4. At 24 weeks of treatment, the patient had an IGA PN-S score of 0 and an average WI-NRS score of 1.3.

FIG. 14 depicts a table of the baseline disease characteristics for patients in the PRIME/EFC16459 and PRIME2/EFC16460 studies. The total enrolled patients in both studies had a mean (SD) WI-NRS of 8.5 (1.0) at baseline.

### DETAILED DESCRIPTION

Before the disclosure is described, it is to be understood that disclosure is not limited to particular methods and experimental conditions described, as such methods and conditions may vary. It is also to be understood that the terminology used herein is for the purpose of describing particular embodiments only, and is not intended to be limiting, because the scope of the disclosure will be limited only by the appended claims.

Unless defined otherwise, all technical and scientific terms used herein have the same meaning as commonly understood by one of ordinary skill in the art to which this disclosure belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of the named disorder or condition.

Although any methods and materials similar or equivalent to those described herein can be used in the practice of the disclosures herein, the typical methods and materials are now described. All publications mentioned herein are incorporated herein by reference in their entirety.

The present disclosure provides methods and compositions for treating prurigo nodularis (PN).

As used herein, “prurigo nodularis” refers to the presence of chronic pruritus for ≥6 weeks, as well as, a history of and/or signs of repeated scratching and multiple localized/generalized pruriginous skin lesions on a subject.

As used herein, “pruriginous skin lesions” refers to papules, nodules and/or plaques on a subject that are whitish, hyperpigmented, or pink.

As used herein, “treating prurigo nodularis” refers to treating one or more of the symptoms of prurigo nodularis, including, but not limited to, decreasing the number of lesions, decreasing the size of lesions, reducing pruritus associated with prurigo nodularis, and the like.

In certain embodiments, a subject with prurigo nodularis has one or more comorbid atopic inflammatory conditions. As used herein, “atopic inflammatory conditions” include, but are not limited to, one or more of allergic rhinitis, allergic fungal rhinosinusitis, chronic sinusitis, allergic bronchopulmonary aspergillosis (ABPA), allergic conjunctivitis, allergic rhinoconjunctivitis, asthma, eosinophilic esophagitis, atopic conjunctivitis, atopic dermatitis, aspirin hypersensitivity, non-steroidal anti-inflammatory drug (NSAID) hypersensitivity (e.g., NSAIDs exacerbated respiratory disease, or NSAID-ERD), perennial allergic rhinitis (PAR), atopic dermatitis (AD), food allergy, hives or urticaria, and exercise induced bronchospasm. In certain exemplary embodiments, a subject with prurigo nodularis has comorbid atopic dermatitis, e.g., mild atopic dermatitis, moderate atopic dermatitis, moderate-to-severe atopic dermatitis or severe atopic dermatitis.

### Methods for Improving PN-Associated Patient-Reported Outcome (PRO) Measures and Clinician-Reported Outcome (ClinRO) Measures

Methods for improving one or more PN-associated patient-reported outcome (PRO) measures in a subject in need thereof, wherein the methods comprise administering a pharmaceutical composition comprising an IL-4R antagonist to the subject, are provided. Methods for improving one or more PN-associated clinician-reported outcome (ClinRO) measures in a subject in need thereof, wherein the methods comprise administering a pharmaceutical composition comprising an IL-4R antagonist to the subject, are provided.

Examples of PN-associated PRO measures include: (1) worst-itch numerical rating scale (WI-NRS), (2) dermatology life quality index (DLQI), (3) pain numeric scale, (4) sleep numeric scale, (5) hospital anxiety and depression scale, (6) patient global impression of change (PGIC), (7) patient global impression of severity (PGIS), and (8) Euroqol-5 dimensions (EQ-5D) score.

An “improvement in a PN-associated PRO measure” means an increase from baseline of one or more of sleep numeric scale score, and Euroqol-5 dimensions (EQ-5D) score and/or a decrease from baseline of one or more of worst-itch numerical rating scale score (WI-NRS), pain numeric scale score, hospital anxiety and depression scale (HADS) score, dermatology life quality index (DLQI) score, patient global impression of change (PGIC) score, and patient global impression of severity (PGIS) score. As used herein, the term “baseline,” with regard to a PN-associated PRO measure, means the numerical value of the PRO measure for a patient prior to or at the time of administration of a pharmaceutical composition comprising an IL-4R antagonist.

Examples of PN-associated ClinRO measures include: (1) investigator's global assessment for prurigo nodularis (IGA PN) and (2) prurigo activity score (PAS).

An “improvement in a PN-associated ClinRO measure” means a decrease from baseline of one or more of investigator's global assessment for prurigo nodularis (IGA PN) score or prurigo activity score (PAS). As used herein, the term “baseline,” with regard to a PN-associated ClinRO measure, means the numerical value of the ClinRO measure for a patient prior to or at the time of administration of a pharmaceutical composition comprising an IL-4R antagonist.

To determine whether an PN-associated parameter has “improved,” the parameter is quantified at baseline and at a time point after administration of the pharmaceutical composition described herein. For example, an PN-associated parameter may be measured at day 1, day 2, day 3, day 4, day 5, day 6, day 7, day 8, day 9, day 10, day 11, day 12, day 14, or at week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with the pharmaceutical composition. The difference between the value of the parameter at a particular time point following initiation of treatment and the value of the parameter at baseline is used to establish whether there has been an “improvement” in the PN-associated parameter (e.g., an increase or decrease, as the case may be, depending on the specific parameter being measured).

The terms “acquire” or “acquiring” as used herein, refer to obtaining possession of a physical entity, or a value, e.g., a numerical value, by “directly acquiring” or “indirectly acquiring” the physical entity or value, such as a PN-associated parameter. “Directly acquiring” means performing a process (e.g., performing a synthetic or analytical method) to obtain the physical entity or value. “Indirectly acquiring” refers to receiving the physical entity or value from another party or source (e.g., a third-party laboratory that directly acquired the physical entity or value.) Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”).

Information that is acquired indirectly can be provided in the form of a report, e.g., supplied in paper or electronic form, such as from an online database or application (an “App”). The report or information can be provided by, for example, a healthcare institution, such as a hospital or clinic; or a healthcare provider, such as a doctor or nurse.

Itch-Free Days: According to certain embodiments, administration of an IL-4R antagonist to a patient results in an increase from baseline in itch-free days experienced by a subject. For example, administration of an IL-4R antagonist to a subject in need thereof causes an increase from baseline in itch-free days experienced by a subject of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days per month.

Number of PN Nodules: According to certain embodiments, administration of an IL-4R antagonist to a patient results in a decrease from baseline in PN nodules (i.e., lesions). For example, administration of an IL-4R antagonist to a subject in need thereof causes a decrease from baseline in PN nodules of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 nodules.

In some embodiments, the patient has a minimum of 20 PN lesions in total on both legs, and/or both arms and/or trunk at baseline before administration of the IL-4R antagonist.

Worst-Itch Numeric Rating Scale: According to some embodiments, administration of an IL-4R antagonist to a patient results in a decrease from baseline of worst-itch numerical rating scale (WI-NRS) score. WI-NRS is a PRO comprised of a single item rated on a scale from 0 (“no itch”) to 10 (“worst imaginable itch”) (Stander S, et al. Serlopitant reduced pruritus in patients with prurigo nodularis in a phase 2, randomized, placebo-controlled trial. J Am Acad Dermatol. 2019; 80(5):1395-402.) Participants are asked to rate the intensity of their worst pruritus (itch) over the past 24 hours using this scale. Daily WI-NRS scores are summed over a 7-day period to create an average weekly WI-NRS score.

Therapeutic methods are provided that result in a decrease in WI-NRS score from baseline. For example, administration of an IL-4R antagonist to a subject in need thereof causes a decrease in WI-NRS score from baseline of about 1, 2, 3, 4, 5, 6, 7, 8, 9, or 10 points. In some embodiments, a subject has a baseline WI-NRS score of equal to or greater than 7 before treatment with the IL-4R antagonist.

Investigator's Global Assessment for Prurigo Nodularis: According to some embodiments, administration of an IL-4R antagonist to a patient results in a decrease from baseline of investigator's global assessment for prurigo nodularis (IGA PN) score. IGA PN is a clinician-reported outcome (ClinRO) that allows clinicians to assess the activity of PN (IGA PN-A) using a 5-point scale from 0 (clear) to 4 (severe); and the stage of the disease (IGA PN-S) using a 5-point scale from 0 (clear) to 4 (severe): 0=clear, no nodules; 1=almost clear, 1-5 nodules; 2=mild, 6-19 nodules; 3=moderate, 20-99 nodules; and 4=severe, ≥100 nodules.

Therapeutic methods are provided that result in a decrease in IGA PN score from baseline. For example, administration of an IL-4R antagonist to a subject in need thereof causes a decrease in IGA PN score from baseline of about 1, 2, 3, or 4 points.

Prurigo Activity Score: According to some embodiments, administration of an IL-4R antagonist to a patient results in a decrease from baseline of prurigo activity score (PAS). The prurigo activity score (PAS) is a ClinRO measurement. The original PAS questionnaire Version 0.9 consists of 7 items, developed by expert clinicians in PN (Polking J, et al. Prurigo Activity Score (PAS): validity and reliability of a new instrument to monitor chronic prurigo. J Eur Acad Dermatol Venereol. 2018; 32(10):1754-60.) The items of the PAS evaluate the pruriginous lesions in terms of: type (visible lesions: Item 1a; predominant lesions: Item 1b); estimated number (Item 2); distribution (Item 3, 4); and size (biggest lesion: Item 6a; representative lesion: Item 6b). Other items evaluate the representative body area and exact number of lesions (Item 5), the activity in terms of percentage of pruriginous lesions with excoriations/crusts on top (reflecting active scratching; Item 7a) and the percentage of healed pruriginous lesions (reflecting healing of chronic prurigo; Item 7b).

Therapeutic methods are provided that result in a decrease in PAS score from baseline.

Dermatology life quality index (DLQI): According to certain embodiments, administration of an IL-4R antagonist to a patient results in a decrease from baseline of the DLQI score. The Dermatology life quality index (DLQI) is a PRO developed to measure dermatology-specific HRQoL in adult participants. (See Finlay A Y, Khan G K. Dermatology life quality index (DLQI): a simple practical measure for routine clinical use. Clin Exp Dermatol. 1994; 19:210-6.) The instrument comprises 10 items assessing the impact of skin disease on participants' health-related quality of life (HRQoL) over the previous week. The items cover symptoms, leisure activities, work/school or holiday time, personal relationships including intimate, the side effects of treatment, and emotional reactions to having a skin disease. It is a validated questionnaire used in clinical practice and clinical trials. (See Chernyshov P V. The evolution of quality of life assessment and use in dermatology. Dermatology. 2019; 235(3):167-74.) Response scale is a 4-point Likert scale (0=“not at all” and 3=“very much”) for 9 items. The remaining 1 item about work/studying asks whether work/study has been prevented and then (if “no”) to what degree the skin condition has been a problem at work/study; the item is rated on a 3-point Likert scale (not at all′ to ‘a lot’). Overall scoring ranges from 0 to 30, with a high score indicative of a poor HRQoL.

Therapeutic methods are provided that result in a decrease in DLQI score from baseline. For example, administration of an IL-4R antagonist to a subject in need thereof causes a decrease in DLQI score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 points.

Pain Numeric Rating Scale: According to certain embodiments, administration of an IL-4R antagonist to a patient results in a decrease from baseline of pain numeric rating scale (NRS) score. In the pain NRS participants are asked to rate their worst skin pain in the past 24 hours using a 0 to 10 numeric rating scale (NRS), with 0=no pain to 10=worst pain possible.

Therapeutic methods are provided that result in a decrease in pain NRS score from baseline. For example, administration of an IL-4R antagonist to a subject in need thereof causes a decrease in pain NRS score from baseline of about 1, 2, 3, 4, 5, 6, 7, 8, 9, or 10 points.

Sleep Numeric Rating Scale: According to certain embodiments, administration of an IL-4R antagonist to a patient results in an increase from baseline of sleep numeric rating scale (NRS) score. In the sleep NRS, participants are asked to rate their sleep quality on their past night upon awakening, using a 0 to 10 NRS, with 0=worst possible sleep and 10=best possible sleep.

Therapeutic methods are provided that result in an increase in sleep NRS score from baseline. For example, administration of an IL-4R antagonist to a subject in need thereof causes an increase in pain NRS score from baseline of about 1, 2, 3, 4, 5, 6, 7, 8, 9, or 10 points.

Hospital Anxiety and Depression Scale: According to certain embodiments, administration of an IL-4R antagonist to a patient results in a decrease from baseline of hospital anxiety and depression scale (HADS) score. The hospital anxiety and depression scale (HADS) is a PRO instrument for screening anxiety and depression in non-psychiatric populations. Repeated administration also provides information about changes to a patient's emotional state. (Zigmond A S, Snaith R P. The hospital anxiety and depression scale. Acta Psychiatr Scand. 1983; 67(6):361-70 and Herrmann C. International experiences with the Hospital Anxiety and Depression Scale—a review of validation data and clinical results. J Psychosom Res. 1997; 42(1):17-41.) The HADS consists of 14 items, 7 each for anxiety and depression symptoms. Possible scores range from 0 to 21 for each subscale. The following cut-off scores are recommended for both subscales: 0 to 7: normal; 8 to 10: borderline abnormal (borderline case); and 11 to 21: abnormal.

Therapeutic methods are provided that result in a decrease in HADS score from baseline. For example, administration of an IL-4R antagonist to a subject in need thereof causes a decrease in HADS score from baseline of about 1, 2, 3, 4, 5, 6, 7, 8, 9, 10, 11, 12, 13, 14, 15, 16, 17, 18, 19, 20, or 21.

Use of Anti-Depressants: According to certain embodiments, administration of an IL-4R antagonist to a patient results in a decrease from baseline of anti-depressant use. Therapeutic methods are provided that result in a decrease or elimination of anti-depressant use from baseline. In certain embodiments, administration of an IL-4R antagonist to a subject in need thereof results in a decrease in anti-depressant use by the subject by about 10%, about 15%, about 20%, about 25%, about 30%, about 35%, about 40%, about 45%, about 50%, about 55%, about 60%, about 65%, about 70%, about 75%, about 80%, about 85%, or about 90% or more. In other embodiments, administration of an IL-4R antagonist to a subject in need thereof results in the elimination of anti-depressant use by the subject.

Patient Global Impression of Change (PGIC): According to certain embodiments, administration of an IL-4R antagonist to a patient results in a decrease from baseline of PGIC score. The patient global impression of change (PGIC) is a 1-item questionnaire that asks the participant to provide the overall self-assessment of change in their PN overall on a 7-point scale, compared to just before participant started taking the study treatment. Response choices are: 0=“very much better,” 1=“moderately better,” 2=“a little better,” 3=“no change,” 4=“a little worse,” 5=“moderately worse,” 6=“very much worse.” (See Guy W et al. ECDEU Assessment Manual for Psychopharmacology. Rockville, Md.: US Department of Health, Education, and Welfare Public Health Service Alcohol, Drug Abuse, and Mental Health Administration, 1976.)

Therapeutic methods are provided that result in a decrease in PGIC score from baseline. For example, administration of an IL-4R antagonist to a subject in need thereof causes a decrease in PGIC score from baseline of about 1, 2, 3, 4, 5, or 6 points.

Patient Global Impression of Severity (PGIS): According to certain embodiments, administration of an IL-4R antagonist to a patient results in a decrease from baseline of PGIS score. The Patient Global Impression of Severity (PGIS) is a 1-item questionnaire that asks participants to provide the overall self-assessment of their disease severity on a 4-point scale for the past week. Response choices are: 1=“none,” 2=“mild,” 3=“moderate,” 4=“severe.” (See Guy W et al. ECDEU Assessment Manual for Psychopharmacology. Rockville, Md.: US Department of Health, Education, and Welfare Public Health Service Alcohol, Drug Abuse, and Mental Health Administration, 1976.)

Therapeutic methods are provided that result in a decrease in PGIS score from baseline. For example, administration of an IL-4R antagonist to a subject in need thereof causes a decrease in PGIS score from baseline of about 1, 2, or 3.

Euroqol-5 dimensions (EQ-5D): According to certain embodiments, administration of an IL-4R antagonist to a patient results in an increase from baseline of EQ-5D. The Euroqol-5 dimensions (EQ-5D) is a standardized PRO measure of health status developed by the EuroQol Group in order to provide a simple, generic measure of health for clinical and economic appraisal. The adult version of the questionnaire is adapted to patients aged 16 and older. The EQ-5D consists of 2 parts: the descriptive system and the EQ visual analogue scale (EQ VAS). The EQ-5D 5L descriptive system comprises the following 5 dimensions: mobility, self-care, usual activities, pain/discomfort, and anxiety/depression. Each dimension has 5 levels of perceived problems: “no problem,” “slight problems,” “moderate problems,” “severe problems,” and “inability to do the activity.” (See Herdman M, et al. Development and preliminary testing of the new five-level version of EQ-5D (EQ-5D-5L). Qual. Life Res. 2011; 20(10):1727-36.) The respondent is asked to indicate his/her health state by ticking (or placing a cross) in the box against the most appropriate statement in each of the 5 dimensions; this results in a 1-digit number expressing the level for that dimension. The digits for 5 dimensions can be combined in a 5-digit number describing the respondent's health state. The EQ VAS records the respondent's self-rated health on a vertical, VAS where the endpoints are labeled “best imaginable health state (100)” and “worst imaginable health state (0).” This information can be used as a quantitative measure of health outcome as judged by the individual respondents.

Therapeutic methods are provided that result in an increase in EQ VAS score from baseline. For example, administration of an IL-4R antagonist to a subject in need thereof causes an increase in EQ VAS score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 points.

### Interleukin-4 Receptor Antagonists

The methods featured herein comprise administering to a subject in need thereof a therapeutic composition comprising an IL-4R antagonist. As used herein, an “IL-4R antagonist” is any agent that binds to or interacts with IL-4R and inhibits the normal biological signaling function of IL-4R when IL-4R is expressed on a cell in vitro or in vivo. Non-limiting examples of categories of IL-4R antagonists include small molecule IL-4R antagonists, anti-IL-4R aptamers, peptide-based IL-4R antagonists (e.g., “peptibody” molecules), and antibodies or antigen-binding fragments of antibodies that specifically bind human IL-4R. According to certain embodiments, the IL-4R antagonist comprises an anti-IL-4R antibody that can be used in the context of the methods described elsewhere herein. For example, in one embodiment, the IL-4R antagonist is an antibody or antigen-binding fragment thereof that specifically binds to an IL-4R, and comprises the heavy chain and light chain (complementarity determining region) CDR sequences from the heavy chain variable region (HCVR) and light chain variable region (LCVR) of SEQ ID NOs:1 and 2, respectively.

The term “human IL4R” (hIL-4R) refers to a human cytokine receptor that specifically binds to interleukin-4 (IL-4), such as IL-4Rα.

The term “antibody” refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2, and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments, the FRs of the anti-IL-4R antibody (or antigen-binding portion thereof) may be identical to the human germline sequences, or may be naturally or artificially modified. An amino acid consensus sequence may be defined based on a side-by-side analysis of two or more CDRs.

The term “antibody” also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds to an antigen to form a complex. Antigen-binding fragments of an antibody may be derived, e.g., from full antibody molecules using any suitable standard techniques, such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment.”

An antigen-binding fragment of an antibody will typically comprise at least one variable domain. The variable domain may be of any size or amino acid composition and will generally comprise at least one CDR that is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody described herein include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (X) V_{L}-C_{H}3; (Xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids that result in a flexible or semi-flexible linkage between adjacent variable and/or constant domains in a single polypeptide molecule, typically the hinge region may consist of between 2 to 60 amino acids, typically between 5 to 50, or typically between 10 to 40 amino acids. Moreover, an antigen-binding fragment of an antibody described herein may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, may be adapted for use in the context of an antigen-binding fragment of an antibody described herein using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody” includes antibodies having variable and constant regions derived from human germline immunoglobulin sequences. The human antibodies described herein may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” does not include antibodies in which CDR sequences derived from the germline of another mammalian species, such as a mouse, have been grafted onto human framework sequences.

The term “recombinant human antibody” includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. Antibodies having one or more mutations in the hinge, C_{H}2, or C_{H}3 region, which may be desirable, for example, in production, to improve the yield of the desired antibody form, are provided.

An “isolated antibody” means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody”. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-4R includes antibodies that bind IL-4R or portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM, or less than about 0.5 nM, as measured in a surface plasmon resonance assay. An isolated antibody that specifically binds human IL-4R may, however, have cross-reactivity to other antigens, such as IL-4R molecules from other (non-human) species.

The anti-IL-4R antibodies useful for the methods may comprise one or more amino acid substitutions, insertions, and/or deletions (e.g., 1, 2, 3, 4, 5, 6, 7, 8, 9, or 10 substitutions and/or 1, 2, 3, 4, 5, 6, 7, 8, 9, or 10 insertions and/or 1, 2, 3, 4, 5, 6, 7, 8, 9, or 10 deletions) in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. Methods involving the use of antibodies, and antigen-binding fragments thereof, that are derived from any of the amino acid sequences disclosed herein, wherein one or more amino acids (e.g., 1, 2, 3, 4, 5, 6, 7, 8, 9, or 10 amino acids) within one or more framework and/or one or more (e.g., 1, 2, 3, 4, 5, 6, 7, 8, 9, 10, 11 or 12 with respect to the tetrameric antibody or 1, 2, 3, 4, 5 or 6 with respect to the HCVR and LCVR of an antibody) CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”), are provided. A person of ordinary skill in the art, starting with the heavy and light chain variable region sequences disclosed herein, can easily produce numerous antibodies and antigen-binding fragments that comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the disclosure.

Methods involving the use of anti-IL-4R antibodies comprising variants of any of the HCVR, LCVR, and/or CDR amino acid sequences disclosed herein having one or more conservative substitutions. For example, the use of anti-IL-4R antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein, are provided.

The term “surface plasmon resonance” refers to an optical phenomenon that allows for the analysis of real-time interactions by detection of alterations in protein concentrations within a biosensor matrix, for example using the BIAcore™ system (Biacore Life Sciences division of GE Healthcare, Piscataway, N.J.).

The term “K_{D}” refers to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, or at least about 96%, 97%, 98% or 99% of the nucleotide bases, as measured by any well-known algorithm of sequence identity, such as FASTA, BLAST or Gap, as discussed below.

As applied to polypeptides, the term “substantial similarity” or “substantially similar” means that two peptide sequences, when optimally aligned, such as by the programs GAP or BESTFIT using default gap weights, share at least 95% sequence identity, or at least 98% or 99% sequence identity. In exemplary embodiments, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. (See, e.g., Pearson (1994) Methods Mol. Biol. 24: 307-331, herein incorporated by reference.) Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Exemplary conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof (See, e.g., GCG Version 6.1.) Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another exemplary algorithm when comparing a sequence of the disclosure to a database containing a large number of sequences from different organisms is the computer program BLAST, especially BLASTP or TBLASTN, using default parameters. (See, e.g., Altschul et al. (1990) J. Mol. Biol. 215:403-410 and Altschul et al. (1997) Nucleic Acids Res. 25:3389-402, each of which is herein incorporated by reference.)

### Preparation of Human Antibodies

Methods for generating human antibodies in transgenic mice are known in the art. Any such known methods can be used to make human antibodies that specifically bind to human IL-4R.

Using VELOCIMMUNE® technology (see, for example, U.S. Pat. No. 6,596,541, Regeneron Pharmaceuticals) or any other known method for generating monoclonal antibodies, high affinity chimeric antibodies to IL-4R are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate a fully human antibody described herein, for example wild-type or modified IgG1 or IgG4. While the constant region selected may vary according to specific use, high affinity antigen-binding and target specificity characteristics reside in the variable region.

In general, the antibodies that can be used in the methods described herein possess high affinities, as described above, when measured by binding to antigen either immobilized on solid phase or in solution phase. The mouse constant regions are replaced with desired human constant regions to generate the fully-human antibodies described herein. While the constant region selected may vary according to specific use, high affinity antigen-binding and target specificity characteristics reside in the variable region.

In one embodiment, human antibody or antigen-binding fragment thereof that specifically binds IL-4R that can be used in the context of the methods described herein comprises the three heavy chain CDRs (HCDR1, HCDR2 and HCDR3) contained within a heavy chain variable region (HCVR) having an amino acid sequence of SEQ ID NO: 1. The antibody or antigen-binding fragment may comprise the three light chain CDRs (LCVR1, LCVR2, LCVR3) contained within a light chain variable region (LCVR) having an amino acid sequence of SEQ ID NO: 2. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In certain embodiments, the antibody or antigen-binding fragment thereof comprises the six CDRs (HCDR1, HCDR2, HCDR3, LCDR1, LCDR2 and LCDR3) from the heavy and light chain variable region amino acid sequence pairs (HCVR/LCVR) of SEQ ID NOs: 1 and 2.

In certain embodiments, the antibody or antigen-binding fragment thereof comprises six CDRs (HCDR1/HCDR2/HCDR3/LCDR1/LCDR2/LCDR3) having the amino acid sequences of SEQ ID NOs: 3/4/5/6/7/8.

In certain embodiments, the antibody or antigen-binding fragment thereof comprises HCVR/LCVR amino acid sequence pair of SEQ ID NOs: 1 and 2.

In certain embodiments, the antibody is dupilumab, which comprises the HCVR/LCVR amino acid sequence pair of SEQ ID NOs: 1 and 2.

In certain embodiments, the antibody sequence is dupilumab, which comprises the heavy chain/light chain amino acid sequence pair of SEQ ID NOs: 9 and 10.

### Dupilumab HCVR Amino Acid Sequence:

(SEQ ID NO: 1)
EVQLVESGGGLEQPGGSLRLSCAGSGFTFRDYAMTWVRQAPGKGLEWVSS
ISGSGGNTYYADSVKGRFTISRDNSKNTLYLQMNSLRAEDTAVYYCAKDR
LSITIRPRYYGLDVWGQGTTVTVS.

### Dupilumab LCVR Amino Acid Sequence:

(SEQ ID NO: 2)
DIVMTQSPLSLPVTPGEPASISCRSSQSLLYSIGYNYLDWYLQKSGQSPQ
LLIYLGSNRASGVPDRFSGSGSGTDFTLKISRVEAEDVGFYYCMQALQTP
YTFGQGTKLEIK.

### Dupilumab HCDR1 Amino Acid Sequence:

| (SEQ ID NO: 3) | |
|---|---|
| | GFTFRDYA. |

### Dupilumab HCDR2 Amino Acid Sequence:

| (SEQ ID NO: 4) | |
|---|---|
| | ISGSGGNT. |

### Dupilumab HCDR3 Amino Acid Sequence:

| (SEQ ID NO: 5) | |
|---|---|
| | AKDRLSITIRPRYYGL. |

### Dupilumab LCDR1 Amino Acid Sequence:

| (SEQ ID NO: 6) | |
|---|---|
| | QSLLYSIGYNY |

### Dupilumab LCDR2 Amino Acid Sequence:

| (SEQ ID NO: 7) | |
|---|---|
| | LGS. |

### Dupilumab LCDR3 Amino Acid Sequence:

| (SEQ ID NO: 8) | |
|---|---|
| | MQALQTPYT |

### Dupilumab HC Amino Acid Sequence:

(SEQ ID NO: 9)
EVQLVESGGGLEQPGGSLRLSCAGSGFTFRDYAMTWVRQAPGKGLEWVSS
ISGSGGNTYYADSVKGRFTISRDNSKNTLYLQMNSLRAEDTAVYYCAKDR
LSITIRPRYYGLDVWGQGTTVTVSSASTKGPSVFPLAPCSRSTSESTAAL
GCLVKDYFPEPVTVSWNSGALTSGVHTFPAVLQSSGLYSLSSVVTVPSSS
LGTKTYTCNVDHKPSNTKVDKRVESKYGPPCPPCPAPEFLGGPSVFLFPP
KPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSL
G (amino acids 1-124 = HCVR; amino acids 125-451 =
HC constant).

### Dupilumab LC Amino Acid Sequence:

(SEQ ID NO: 10)
DIVMTQSPLSLPVTPGEPASISCRSSQSLLYSIGYNYLDWYLQKSGQSPQ
LLIYLGSNRASGVPDRFSGSGSGTDFTLKISRVEAEDVGFYYCMQALQTP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC (amino acids 1-112 = LCVR;
amino acids 112-219 = LC constant).

In certain embodiments, an antibody or antigen-binding fragment thereof of the disclosure comprises light chain variable region (LCVR) and heavy chain variable region (HCVR) sequence pairs (LCVR/HCVR) selected from the group consisting of SCB-VL-39/SCB-VH-92; SCB-VL-40/SCB-VH-92; SCB-VL-41/SCB-VH-92; SCB-VL-42/SCB-VH-92; SCB-VL-43/SCB-VH-92; SCB-VL-44/SCB-VH-92; SCB-VL-44/SCB-VH-62; SCB-VL-44/SCB-VH-68; SCB-VL-44/SCB-VH-72; SCB-VL-44/SCB-VH-82; SCB-VL-44/SCB-VH-85; SCB-VL-44/SCB-VH-91; SCB-VL-44/SCB-VH-93; SCB-VL-45/SCB-VH-92; SCB-VL-46/SCB-VH-92; SCB-VL-47/SCB-VH-92; SCB-VL-48/SCB-VH-92; SCB-VL-49/SCB-VH-92; SCB-VL-50/SCB-VH-92; SCB-VL-51/SCB-VH-92; SCB-VL-51/SCB-VH-93; SCB-VL-52/SCB-VH-92; SCB-VL-52/SCB-VH-62; SCB-VL-52/SCB-VH-91; SCB-VL-53/SCB-VH-92; SCB-VL-54/SCB-VH-92; SCB-VL-54/SCB-VH-62; SCB-VL-54/SCB-VH-68; SCB-VL-54/SCB-VH-72; SCB-VL-54/SCB-VH-82; SCB-VL-54/SCB-VH-85; SCB-VL-54/SCB-VH-91; SCB-VL-55/SCB-VH-92; SCB-VL-55/SCB-VH-62; SCB-VL-55/SCB-VH-68; SCB-VL-55/SCB-VH-72; SCB-VL-55/SCB-VH-82; SCB-VL-55/SCB-VH-85; SCB-VL-55/SCB-VH-91; SCB-VL-56/SCB-VH-92; SCB-VL-57/SCB-VH-92; SCB-VL-57/SCB-VH-93; SCB-VL-57/SCB-VH-59; SCB-VL-57/SCB-VH-60; SCB-VL-57/SCB-VH-61; SCB-VL-57/SCB-VH-62; SCB-VL-57/SCB-VH-63; SCB-VL-57/SCB-VH-64; SCB-VL-57/SCB-VH-65; SCB-VL-57/SCB-VH-66; SCB-VL-57/SCB-VH-67; SCB-VL-57/SCB-VH-68; SCB-VL-57/SCB-VH-69; SCB-VL-57/SCB-VH-70; SCB-VL-57/SCB-VH-71; SCB-VL-57/SCB-VH-72; SCB-VL-57/SCB-VH-73; SCB-VL-57/SCB-VH-74; SCB-VL-57/SCB-VH-75; SCB-VL-57/SCB-VH-76; SCB-VL-57/SCB-VH-77; SCB-VL-57/SCB-VH-78; SCB-VL-57/SCB-VH-79; SCB-VL-57/SCB-VH-80; SCB-VL-57/SCB-VH-81; SCB-VL-57/SCB-VH-82; SCB-VL-57/SCB-VH-83; SCB-VL-57/SCB-VH-84; SCB-VL-57/SCB-VH-85; SCB-VL-57/SCB-VH-86; SCB-VL-57/SCB-VH-87; SCB-VL-57/SCB-VH-88; SCB-VL-57/SCB-VH-89; SCB-VL-57/SCB-VH-90; SCB-VL-57/SCB-VH-91; SCB-VL-58/SCB-VH-91; SCB-VL-58/SCB-VH-92; and SCB-VL-58/SCB-VH-93.

In certain embodiments, an antibody or antigen-binding fragment thereof of the disclosure comprises a LCVR/HCVR sequence pair of SCB-VL-44/SCB-VH-92.

In certain embodiments, an antibody or antigen-binding fragment thereof of the disclosure comprises a LCVR/HCVR sequence pair of SCB-VL-54/SCB-VH-92.

In certain embodiments, an antibody or antigen-binding fragment thereof of the disclosure comprises a LCVR/HCVR sequence pair of SCB-VL-55/SCB-VH-92.

In certain embodiments, an antibody or antigen-binding fragment thereof of the disclosure comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 of SCB-55-LCDR1, and LCDR2 of SCB-55-LCDR2, and an LCDR3 of SCB-55-LCDR3.

In certain embodiments, an antibody or antigen-binding fragment thereof of the disclosure comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 of SCB-55-LCDR1, and LCDR2 of SCB-54-LCDR2, and an LCDR3 of SCB-55-LCDR3.

In certain embodiments, an antibody or antigen-binding fragment thereof of the disclosure comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 of SCB-55-LCDR1, and LCDR2 of SCB-54-LCDR2, and an LCDR3 of SCB-44-LCDR3.

The antibodies recited below in Table 1 are described in more detail in U.S. Pat. No. 10,774,141, incorporated herein by reference in its entirety for all purposes.

TABLE 1

| Sequence ID | Sequence |
|---|---|
| SCB-VL-39 | EIVLTQSPGTLSLSPGERATLSCRASQSVSNSYLAWYQQKPGQAPR |
| SEQ ID NO. 11 | LLIFGASSRATGIPDRFSGSGSGTDFTLTISRLEPEDFAVYYCQQY |
| | GSSPPWTFGQGTKVEIK |
| SCB-VL-40 | EIVLTQSPGTLSLSPGERATLSCRASQSVSSSYLAWYQQKPGQAPR |
| SEQ ID NO. 12 | LLIYGASSRATGIPDRFSGSGSGTDFTLTISRLEPEDFAVYYCQQY |
| | GSSPPWTFGQGTKVEIK |
| SCB-VL-41 | EIVLTQSPGTLSLSPGERATLSCRASQSVSSSYLAWYQQKPGQAPR |
| SEQ ID NO. 13 | LLIFGASSRAPGIPDRFSGSGSGTDFTLTISRLEPEDFAVYYCQQY |
| | GSSPPWTFGQGTKVEIK |
| SCB-VL-42 | EIVLTQSPGTLSLSPGERATLSCRASQSVSNSYLAWYQQKPGQAPR |
| SEQ ID NO. 14 | LLIYGASSRATGIPDRFSGSGSGTDFTLTISRLEPEDFAVYYCQQY |
| | GSSPPWTFGQGTKVEIK |
| SCB-VL-43 | EIVLTQSPGTLSLSPGERATLSCRASQSVSNSYLAWYQQKPGQAPR |
| SEQ ID NO. 15 | LLIFGASSRAPGIPDRFSGSGSGTDFTLTISRLEPEDFAVYYCQQY |
| | GSSPPWTFGQGTKVEIK |
| SCB-VL-44 | EIVLTQSPGTLSLSPGERATLSCRASQSVSSSYLAWYQQKPGQAPR |
| SEQ ID NO. 16 | LLIYGASSRAPGIPDRFSGSGSGTDFTLTISRLEPEDFAVYYCQQY |
| | GSSPPWTFGQGTKVEIK |
| SCB-VL-45 | EIVLTQSPGTLSLSPGERATLSCRASQSVSSSYLAWYQQKPGQAPR |
| SEQ ID NO. 17 | LLIFGASSRATGIPDRFSGSGSGTDFTLTISRLEPEDFAVYYCQQY |
| | DHSPPWTFGQGTKVEIK |
| SCB-VL-46 | EIVLTQSPGTLSLSPGERATLSCRASQSVSSSYLAWYQQKPGQAPR |
| SEQ ID NO. 18 | LLIFGASSRATGIPDRFSGSGSGTDFTLTISRLEPEDFAVYYCQQY |
| | GSSAGWTFGQGTKVEIK |
| SCB-VL-47 | EIVLTQSPGTLSLSPGERATLSCRASQSVSSSYLAWYQQKPGQAPR |
| SEQ ID NO. 19 | LLIFGASSRATGIPDRFSGSGSGTDFTLTISRLEPEDFAVYYCQQY |
| | DHSAGWTFGQGTKVEIK |
| SCB-VL-48 | EIVLTQSPGTLSLSPGERATLSCRASQSVSNSYLAWYQQKPGQAPR |
| SEQ ID NO. 20 | LLIFGASSRATGIPDRFSGSGSGTDFTLTISRLEPEDFAVYYCQQY |
| | DHSPPWTFGQGTKVEIK |
| SCB-VL-49 | EIVLTQSPGTLSLSPGERATLSCRASQSVSSSYLAWYQQKPGQAPR |
| SEQ ID NO. 21 | LLIYGASSRATGIPDRFSGSGSGTDFTLTISRLEPEDFAVYYCQQY |
| | DHSPPWTFGQGTKVEIK |
| SCB-VL-50 | EIVLTQSPGTLSLSPGERATLSCRASQSVSSSYLAWYQQKPGQAPR |
| SEQ ID NO. 22 | LLIFGASSRAPGIPDRFSGSGSGTDFTLTISRLEPEDFAVYYCQQY |
| | DHSPPWTFGQGTKVEIK |
| SCB-VL-51 | EIVLTQSPGTLSLSPGERATLSCRASQSVSSSYLAWYQQKPGQAPR |
| SEQ ID NO. 23 | LLIYGASSRAPGIPDRFSGSGSGTDFTLTISRLEPEDFAVYYCQQY |
| | DHSAGWTFGQGTKVEIK |
| SCB-VL-52 | EIVLTQSPGTLSLSPGERATLSCRASQSVSNSYLAWYQQKPGQAPR |
| SEQ ID NO. 24 | LLIFGASSRAPGIPDRFSGSGSGTDFTLTISRLEPEDFAVYYCQQY |
| | DHSAGWTFGQGTKVEIK |
| SCB-VL-53 | EIVLTQSPGTLSLSPGERATLSCRASQSVSNSYLAWYQQKPGQAPR |
| SEQ ID NO. 25 | LLIYGASSRATGIPDRFSGSGSGTDFTLTISRLEPEDFAVYYCQQY |
| | DHSAGWTFGQGTKVEIK |
| SCB-VL-54 | EIVLTQSPGTLSLSPGERATLSCRASQSVSSSYLAWYQQKPGQAPR |
| SEQ ID NO. 26 | LLIFGASSRAPGIPDRFSGSGSGTDFTLTISRLEPEDFAVYYCQQY |
| | DHSAGWTFGQGTKVEIK |
| SCB-VL-55 | EIVLTQSPGTLSLSPGERATLSCRASQSVSSSYLAWYQQKPGQAPR |
| SEQ ID NO. 27 | LLIYGASSRATGIPDRFSGSGSGTDFTLTISRLEPEDFAVYYCQQY |
| | DHSAGWTFGQGTKVEIK |
| SCB-VL-56 | EIVLTQSPGTLSLSPGERATLSCRASQSVSNSYLAWYQQKPGQAPR |
| SEQ ID NO. 28 | LLIFGASSRATGIPDRFSGSGSGTDFTLTISRLEPEDFAVYYCQQY |
| | DHSAGWTFGQGTKVEIK |
| SCB-VL-57 | EIVLTQSPGTLSLSPGERATLSCRASQSVSSSYLAWYQQKPGQAPR |
| SEQ ID NO. 29 | LLIFGASSRATGIPDRFSGSGSGTDFTLTISRLEPEDFAVYYCQQY |
| | GSSPPWTFGQGTKVEIK |
| SCB-VL-58 | EIVLTQSPGTLSLSPGERATLSCRASQSVSNSYLAWYQQKPGQAPR |
| SEQ ID NO. 30 | LLIYGASSRAPGIPDRFSGSGSGTDFTLTISRLEPEDFAVYYCQQY |
| | DHSAGWTFGQGTKVEIK |
| SCB-VH-59 | EVQLVESGGGLVHPGGSLRLSCAGSGFTFSRNAMFWVRQAPGKGLE |
| SEQ ID NO. 31 | WVSGIGTGGATNYADSVKGRFTISRDNAKNSLYLQMNSLRAEDMAV |
| | YYCARGRYYFDYWGQGTLVTVSS |
| SCB-VH-60 | EVQLVQSGGGLVQPGGSLRLSCAGSGFTFSRNAMFWVRQAPGKGLE |
| SEQ ID NO. 32 | WVSGIGTGGATNYADSVKGRFTISRDNAKNSLYLQMNSLRAEDMAV |
| | YYCARGRYYFDYWGQGTLVTVSS |
| SCB-VH-61 | EVQLVQSGGGLVHPGGSLRLSCAASGFTFSRNAMFWVRQAPGKGLE |
| SEQ ID NO. 33 | WVSGIGTGGATNYADSVKGRFTISRDNAKNSLYLQMNSLRAEDMAV |
| | YYCARGRYYFDYWGQGTLVTVSS |
| SCB-VH-62 | EVQLVQSGGGLVHPGGSLRLSCAGSGFTFSRNAMFWVRQAPGKGLE |
| SEQ ID NO. 34 | WVSGIGTGGATSYADSVKGRFTISRDNAKNSLYLQMNSLRAEDMAV |
| | YYCARGRYYFDYWGQGTLVTVSS |
| SCB-VH-63 | EVQLVQSGGGLVHPGGSLRLSCAGSGFTFSRNAMFWVRQAPGKGLE |
| SEQ ID NO. 35 | WVSGIGTGGATNYADSVKGRFTISRDNAKNSLYLQMNSLRAEDTAV |
| | YYCARGRYYFDYWGQGTLVTVSS |
| SCB-VH-64 | EVQLVESGGGLVQPGGSLRLSCAGSGFTFSRNAMFWVRQAPGKGLE |
| SEQ ID NO. 36 | WVSGIGTGGATNYADSVKGRFTISRDNAKNSLYLQMNSLRAEDMAV |
| | YYCARGRYYFDYWGQGTLVTVSS |
| SCB-VH-65 | EVQLVESGGGLVHPGGSLRLSCAASGFTFSRNAMFWVRQAPGKGLE |
| SEQ ID NO. 37 | WVSGIGTGGATNYADSVKGRFTISRDNAKNSLYLQMNSLRAEDMAV |
| | YYCARGRYYFDYWGQGTLVTVSS |
| SCB-VH-66 | EVQLVQSGGGLVQPGGSLRLSCAASGFTFSRNAMFWVRQAPGKGLE |
| SEQ ID NO. 38 | WVSGIGTGGATNYADSVKGRFTISRDNAKNSLYLQMNSLRAEDMAV |
| | YYCARGRYYFDYWGQGTLVTVSS |
| SCB-VH-67 | EVQLVQSGGGLVHPGGSLRLSCAGSGFTFSRNAMFWVRQAPGKGLE |
| SEQ ID NO. 39 | WVSGIGTGGATSYADSVKGRFTISRDNAKNSLYLQMNSLRAEDTAV |
| | YYCARGRYYFDYWGQGTLVTVSS |
| SCB-VH-68 | EVQLVQSGGGLVHPGGSLRLSCAGSGFTFSRNAMFWVRQAPGKGLE |
| SEQ ID NO. 40 | WVSGIGTGGATNYADSVKGRFTISRDNAKNSLYLQMNSLRAEDMAV |
| | YYCARGRYYFPWWGQGTLVTVSS |
| SCB-VH-69 | EVQLVESGGGLVHPGGSLRLSCAGSGFTFSRNAMFWVRQAPGKGLE |
| SEQ ID NO. 41 | WVSGIGTGGATNYADSVKGRFTISRDNAKNSLYLQMNSLRAEDMAV |
| | YYCARGRYYFPWWGQGTLVTVSS |
| SCB-VH-70 | EVQLVQSGGGLVQPGGSLRLSCAGSGFTFSRNAMFWVRQAPGKGLE |
| SEQ ID NO. 42 | WVSGIGTGGATNYADSVKGRFTISRDNAKNSLYLQMNSLRAEDMAV |
| | YYCARGRYYFPWWGQGTLVTVSS |
| SCB-VH-71 | EVQLVQSGGGLVHPGGSLRLSCAASGFTFSRNAMFWVRQAPGKGLE |
| SEQ ID NO. 43 | WVSGIGTGGATNYADSVKGRFTISRDNAKNSLYLQMNSLRAEDMAV |
| | YYCARGRYYFPWWGQGTLVTVSS |
| SCB-VH-72 | EVQLVQSGGGLVHPGGSLRLSCAGSGFTFSRNAMFWVRQAPGKGLE |
| SEQ ID NO. 44 | WVSGIGTGGATSYADSVKGRFTISRDNAKNSLYLQMNSLRAEDMAV |
| | YYCARGRYYFPWWGQGTLVTVSS |
| SCB-VH-73 | EVQLVQSGGGLVHPGGSLRLSCAGSGFTFSRNAMFWVRQAPGKGLE |
| SEQ ID NO. 45 | WVSGIGTGGATNYADSVKGRFTISRDNAKNSLYLQMNSLRAEDTAV |
| | YYCARGRYYFPWWGQGTLVTVSS |
| SCB-VH-74 | EVQLVQSGGGLVHPGRSLRLSCAGSGFTFSRNAMFWVRQAPGKGLE |
| SEQ ID NO. 46 | WVSGIGTGGATNYADSVKGRFTISRDNAKNSLYLQMNSLRAEDMAV |
| | YYCARGRYYFDYWGQGTLVTVSS |
| SCB-VH-75 | EVQLVQSGGGLVHPGGSLRLTCAGSGFTFSRNAMFWVRQAPGKGLE |
| SEQ ID NO. 47 | WVSGIGTGGATNYADSVKGRFTISRDNAKNSLYLQMNSLRAEDMAV |
| | YYCARGRYYFDYWGQGTLVTVSS |
| SCB-VH-76 | EVQLVQSGGGLVHPGGSLRLSCAGSGFTFSRNAMHWVRQAPGKGLE |
| SEQ ID NO. 48 | WVSGIGTGGATNYADSVKGRFTISRDNAKNSLYLQMNSLRAEDMAV |
| | YYCARGRYYFDYWGQGTLVTVSS |
| SCB-VH-77 | EVQLVQSGGGLVHPGGSLRLSCAGSGFTFSRNAMFWVRQAPGEGLE |
| SEQ ID NO. 49 | WVSGIGTGGATNYADSVKGRFTISRDNAKNSLYLQMNSLRAEDMAV |
| | YYCARGRYYFDYWGQGTLVTVSS |
| SCB-VH-78 | EVQLVQSGGGLVHPGGSLRLSCAGSGFTFSRNAMFWVRQAPGKGLE |
| SEQ ID NO. 50 | WVSGIGTGGATNYADSVKGRFTISRDEAKNSLYLQMNSLRAEDMAV |
| | YYCARGRYYFDYWGQGTLVTVSS |
| SCB-VH-79 | EVQLVQSGGGLVHPGGSLRLSCAGSGFTFSRNAMFWVRQAPGKGLE |
| SEQ ID NO. 51 | WVSGIGTGGATNYADSVKGRFTISRDNAKNSLYLQMNSLRAGDMAV |
| | YYCARGRYYFDYWGQGTLVTVSS |
| SCB-VH-80 | EVQLVQSGGGLVHPGGSLRLSCAGSGFTFDDYAMFWVRQAPGKGLE |
| SEQ ID NO. 52 | WVSGIGTGGATNYADSVKGRFTISRDNAKNSLYLQMNSLRAEDMAV |
| | YYCARGRYYFDYWGQGTLVTVSS |
| SCB-VH-81 | EVQLVQSGGGLVQPGGSLRLSCAASGFTFSRNAMFWVRQAPGKGLE |
| SEQ ID NO. 53 | WVSGIGTGGATSYADSVKGRFTISRDNAKNSLYLQMNSLRAEDTAV |
| | YYCARGRYYFPWWGQGTLVTVSS |
| SCB-VH-82 | EVQLVESGGGLVHPGGSLRLSCAASGFTFSRNAMFWVRQAPGKGLE |
| SEQ ID NO. 54 | WVSGIGTGGATSYADSVKGRFTISRDNAKNSLYLQMNSLRAEDTAV |
| | YYCARGRYYFPWWGQGTLVTVSS |
| SCB-VH-83 | EVQLVESGGGLVQPGGSLRLSCAGSGFTFSRNAMFWVRQAPGKGLE |
| SEQ ID NO. 55 | WVSGIGTGGATSYADSVKGRFTISRDNAKNSLYLQMNSLRAEDTAV |
| | YYCARGRYYFPWWGQGTLVTVSS |
| SCB-VH-84 | EVQLVESGGGLVQPGGSLRLSCAASGFTFSRNAMFWVRQAPGKGLE |
| SEQ ID NO. 56 | WVSGIGTGGATNYADSVKGRFTISRDNAKNSLYLQMNSLRAEDTAV |
| | YYCARGRYYFPWWGQGTLVTVSS |
| SCB-VH-85 | EVQLVESGGGLVQPGGSLRLSCAASGFTFSRNAMFWVRQAPGKGLE |
| SEQ ID NO. 57 | WVSGIGTGGATSYADSVKGRFTISRDNAKNSLYLQMNSLRAEDMAV |
| | YYCARGRYYFPWWGQGTLVTVSS |
| SCB-VH-86 | EVQLVQSGGGLVHPGGSLRLSCAASGFTFSRNAMFWVRQAPGKGLE |
| SEQ ID NO. 58 | WVSGIGTGGATSYADSVKGRFTISRDNAKNSLYLQMNSLRAEDTAV |
| | YYCARGRYYFPWWGQGTLVTVSS |
| SCB-VH-87 | EVQLVQSGGGLVQPGGSLRLSCAGSGFTFSRNAMFWVRQAPGKGLE |
| SEQ ID NO. 59 | WVSGIGTGGATSYADSVKGRFTISRDNAKNSLYLQMNSLRAEDTAV |
| | YYCARGRYYFPWWGQGTLVTVSS |
| SCB-VH-88 | EVQLVESGGGLVHPGGSLRLSCAGSGFTFSRNAMFWVRQAPGKGLE |
| SEQ ID NO. 60 | WVSGIGTGGATSYADSVKGRFTISRDNAKNSLYLQMNSLRAEDTAV |
| | YYCARGRYYFPWWGQGTLVTVSS |
| SCB-VH-89 | EVQLVQSGGGLVHPGGSLRLSCAGSGFTFSRNAMFWVRQAPGKGLE |
| SEQ ID NO. 61 | WVSGIGTGGATSYADSVKGRFTISRDNAKNSLYLQMNSLRAEDTAV |
| | YYCARGRYYFPWWGQGTLVTVSS |
| SCB-VH-90 | EVQLVESGGGLVQPGGSLRLSCAASGFTFSRNAMFWVRQAPGKGLE |
| SEQ ID NO. 62 | WVSGIGTGGATNYADSVKGRFTISRDNAKNSLYLQMNSLRAEDMAV |
| | YYCARGRYYFPWWGQGTLVTVSS |
| SCB-VH-91 | EVQLVESGGGLVQPGGSLRLSCAASGFTFSRNAMFWVRQAPGKGLE |
| SEQ ID NO. 63 | WVSGIGTGGATSYADSVKGRFTISRDNAKNSLYLQMNSLRAEDTAV |
| | YYCARGRYYFDYWGQGTLVTVSS |
| SCB-VH-92 | EVQLVQSGGGLVHPGGSLRLSCAGSGFTFSRNAMFWVRQAPGKGLE |
| SEQ ID NO. 64 | WVSGIGTGGATNYADSVKGRFTISRDNAKNSLYLQMNSLRAEDMAV |
| | YYCARGRYYFDYWGQGTLVTVSS |
| SCB-VH-93 | EVQLVESGGGLVQPGGSLRLSCAASGFTFSRNAMFWVRQAPGKGLE |
| SEQ ID NO. 65 | WVSGIGTGGATSYADSVKGRFTISRDNAKNSLYLQMNSLRAEDTAV |
| | YYCARGRYYFPWWGQGTLVTVSS |
| SCB-92-HCDR1 | RNAMF |
| SEQ ID NO. 66 | |
| SCB-92-HCDR3 | GIGTGGATSYADSVKG |
| SEQ ID NO. 67 | |
| SCB-92-HCDR3 | GRYYFDY |
| SEQ ID NO. 68 | |
| SCB-55-LCDR1 | RASQSVSSSYLA |
| SEQ ID NO. 69 | |
| SCB-55-LCDR2 | GASSRAT |
| SEQ ID NO. 70 | |
| SCB-55-LCDR3 | QQYDHSAGWT |
| SEQ ID NO. 71 | |
| SCB-54-LCDR2 | GASSRAP |
| SEQ ID NO. 72 | |
| SCB-44-LCDR3 | QQYGSSPPWT |
| SEQ ID NO. 73 | |

In certain embodiments, an antibody or antigen-binding fragment thereof of the disclosure comprises light chain variable region (LCVR) and heavy chain variable region (HCVR) sequence pairs (LCVR/HCVR) selected from the group consisting of MEDI-1-VL/MEDI-1-VH through MEDI-42-VL/MEDI-42-VH.

In certain embodiments, an antibody or antigen-binding fragment thereof of the disclosure comprises a LCVR/HCVR sequence pair of MEDI-37GL-VL/MEDI-37GL-VH.

In certain embodiments, an antibody or antigen-binding fragment thereof of the disclosure comprises an HCVR comprising an HCDR1 sequence of MEDI-37GL-HCDR1, an HCDR2 sequence of MEDI-37GL-HCDR2, and an HCDR3 sequence of MEDI-37GL-HCDR3, and an LCVR comprising an LCDR1 of MEDI-37GL-LCDR1, and LCDR2 of MEDI-37GL-LCDR2, and an LCDR3 of MEDI-37GL-LCDR3.

The antibodies recited below in Table 2 are described in more detail in U.S. Pat. No. 8,877,189, incorporated herein by reference in its entirety for all purposes.

TABLE 2

| Sequence ID | Sequence |
|---|---|
| MEDI-1-VH | QVQLVQSGAEVKKPGASVKVSCKASGYAFTSYYMHWARQAPGQGL |
| SEQ ID NO. 74 | EWMGIINPSGGSTSYAQKFQGRVTMTRDTSTSTVYMELSSLRSED |
| | TAVYYCARGKWWLDYWGKGTLVTVSS |
| MEDI-1-VL | QSVLTQPPSVSAAPGQKVTISCSGGSSNIGNSYVSWYQQLPGTAP |
| SEQ ID NO. 75 | KLLIYDNNKRPSGIPDRFSGSKSGTSATLAITGLQTGDEADYYCG |
| | TWDTSLSANYVFGTGTKLTVL |
| MEDI-2-VH | QVQLVQSGAEVKKPGASVKVSCKASGYAFTSYYMHWARQAPGQGL |
| SEQ ID NO. 76 | EWMGIINPSGGSTSYAQKFQGRVTMTRDTSTSTVYMELSSLRSED |
| | TAVYYCARGKWWLYNWGKGTLVTVSS |
| MEDI-2-VL | QSVLTQPPSVSAAPGQKVTISCSGGSSNIGNSYVSWYQQLPGTAP |
| SEQ ID NO. 77 | KLLIYDNNKRPSGIPDRFSGSKSGTSATLAITGLQTGDEADYYCG |
| | TWDTSQPPNPLFGTGTKLTVL |
| MEDI-3-VH | QVQLVQSGAEVKKPGASVKVSCKASGYAFTSYYMHWARQAPGQGL |
| SEQ ID NO. 78 | EWMGIINPSGGSTSYAQKFQGRVTMTRDTSTSTVYMELSSLRSED |
| | TAVYYCARGKLLKNPWGKGTLVTVSS |
| MEDI-3-VL | QSVLTQPPSVSAAPGQKVTISCSGGSSNIGNSYVSWYQQLPGTAP |
| SEQ ID NO. 79 | KLLIYDNNKRPSGIPDRFSGSKSGTSATLAITGLQTGDEADYYCG |
| | TWFGTPASNYVFGTGTKLTVL |
| MEDI-4-VH | QVQLVQSGAEVKKPGASVKVSCKASGYAFTSYYMHWARQAPGQGL |
| SEQ ID NO. 80 | EWMGIINPSGGSTSYAQKFQGRVTMTRDTSTSTVYMELSSLRSED |
| | TAVYYCARGKWWLYNWGKGTLVTVSS |
| MEDI-4-VL | QSVLTQPPSVSAAPGQKVTISCSGGSSNIGNSYVSWYQQLPGTAP |
| SEQ ID NO. 81 | KLLIYDNNKRPSGIPDRFSGSKSGTSATLAITGLQTGDEADYYCG |
| | TWDTSSPPQPIFGTGTKLTVL |
| MEDI-5-VH | QVQLVQSGAEVKKPGASVKVSCKASGYAFTSYYMHWARQAPGQGL |
| SEQ ID NO. 82 | EWMGIINPSGGSTSYAQKFQGRVTMTRDTSTSTVYMELSSLRSED |
| | TAVYYCARGKWWLYDWGKGTLVTVSS |
| MEDI-5-VL | QSVLTQPPSVSAAPGQKVTISCSGGSSNIGNSYVSWYQQLPGTAP |
| SEQ ID NO. 83 | KLLIYDNNKRPSGIPDRFSGSKSGTSATLAITGLQTGDEADYYCG |
| | TWDTSSPPQPIFGTGTKLTVL |
| MEDI-6-VH | QVQLVQSGAEVKKPGASVKVSCKASGYAFTSYYMHWARQAPGQGL |
| SEQ ID NO. 84 | EWMGIINPSGGSTSYAQKFQGRVTMTRDTSTSTVYMELSSLRSED |
| | TAVYYCARGKYWMYDWGKGTLVTVSS |
| MEDI-6-VL | QSVLTQPPSVSAAPGQKVTISCSGGSSNIGNSYVSWYQQLPGTAP |
| SEQ ID NO. 85 | KLLIYDNNKRPSGIPDRFSGSKSGTSATLAITGLQTGDEADYYCG |
| | TWDTSTTYHPIFGTGTKLTVL |
| MEDI-7-VH | QVQLVQSGAEVKKPGASVKVSCKASGYAFTSYYMHWARQAPGQGL |
| SEQ ID NO. 86 | EWMGIINPSGGSTSYAQKFQGRVTMTRDTSTSTVYMELSSLRSED |
| | TAVYYCARGKWWWQYWGKGTLVTVSS |
| MEDI-7-VL | QSVLTQPPSVSAAPGQKVTISCSGGSSNIGNSYVSWYQQLPGTAP |
| SEQ ID NO. 87 | KLLIYDNNKRPSGIPDRFSGSKSGTSATLAITGLQTGDEADYYCG |
| | TWDTSSPPQPIFGTGTKLTVL |
| MEDI-8-VH | QVQLVQSGAEVKKPGASVKVSCKASGYAFTSYYMHWARQAPGQGL |
| SEQ ID NO. 88 | EWMGIINPSGGSTSYAQKFQGRVTMTRDTSTSTVYMELSSLRSED |
| | TAVYYCARGKWWWQYWGKGTLVTVSS |
| MEDI-8-VL | QSVLTQPPSVSAAPGQKVTISCSGGSSNIGNSYVSWYQQLPGTAP |
| SEQ ID NO. 89 | KLLIYDNNKRPSGIPDRFSGSKSGTSATLAITGLQTGDEADYYCG |
| | TWDTSTTYHPIFGTGTKLTVL |
| MEDI-9-VH | QVQLVQSGAEVKKPGASVKVSCKASGYAFTSYYMHWARQAPGQGL |
| SEQ ID NO. 90 | EWMGIINPSGGSTSYAQKFQGRVTMTRDTSTSTVYMELSSLRSED |
| | TAVYYCARGKWWLYNWGKGTLVTVSS |
| MEDI-9-VL | QSVLTQPPSVSAAPGQKVTISCSGGSSNIGNSYVSWYQQLPGTAP |
| SEQ ID NO. 91 | KLLIYDNNKRPSGIPDRFSGSKSGTSATLAITGLQTGDEADYYCG |
| | TWDTSTTMYPLFGTGTKLTVL |
| MEDI-10-VH | QVQLVQSGAEVKKPGASVKVSCKASGYAFTSYYMHWARQAPGQGL |
| SEQ ID NO. 92 | EWMGIINPSGGSTSYAQKFQGRVTMTRDTSTSTVYMELSSLRSED |
| | TAVYYCARGKWWLYDWGKGTLVTVSS |
| MEDI-10-VL | QSVLTQPPSVSAAPGQKVTISCSGGSSNIGNSYVSWYQQLPGTAP |
| SEQ ID NO. 93 | KLLIYDNNKRPSGIPDRFSGSKSGTSATLAITGLQTGDEADYYCG |
| | TWDTSTVLTPIFGTGTKLTVL |
| MEDI-11-VH | QVQLVQSGAEVKKPGASVKVSCKASGYAFTSYYMHWARQAPGQGL |
| SEQ ID NO. 94 | EWMGIINPSGGSTSYAQKFQGRVTMTRDTSTSTVYMELSSLRSED |
| | TAVYYCARGKWWFYDWGKGTLVTVSS |
| MEDI-11-VL | QSVLTQPPSVSAAPGQKVTISCSGGSSNIGNSYVSWYQQLPGTAP |
| SEQ ID NO. 95 | KLLIYDNNKRPSGIPDRFSGSKSGTSATLAITGLQTGDEADYYCG |
| | TWDTSPSMIPLFGTGTKLTVL |
| MEDI-12-VH | QVQLVQSGAEVKKPGASVKVSCKASGYAFTSYYMHWARQAPGQGL |
| SEQ ID NO. 96 | EWMGIINPSGGSTSYAQKFQGRVTMTRDTSTSTVYMELSSLRSED |
| | TAVYYCARGKWWFYDWGKGTLVTVSS |
| MEDI-12-VL | QSVLTQPPSVSAAPGQKVTISCSGGSSNIGNSYVSWYQQLPGTAP |
| SEQ ID NO. 97 | KLLIYDNNKRPSGIPDRFSGSKSGTSATLAITGLQTGDEADYYCG |
| | TWDTSTTMYPLFGTGTKLTVL |
| MEDI-13-VH | QVQLVQSGAEVKKPGASVKVSCKASGYAFTSYYMHWARQAPGQGL |
| SEQ ID NO. 98 | EWMGIINPSGGSTSYAQKFQGRVTMTRDTSTSTVYMELSSLRSED |
| | TAVYYCARGKWWLYDWGKGTLVTVSS |
| MEDI-13-VL | QSVLTQPPSVSAAPGQKVTISCSGGSSNIGNSYVSWYQQLPGTAP |
| SEQ ID NO. 99 | KLLIYDNNKRPSGIPDRFSGSKSGTSATLAITGLQTGDEADYYCG |
| | TWDTSTTLQPLFGTGTKLTVL |
| MEDI-14-VH | QVQLVQSGAEVKKPGASVKVSCKASGYAFTSYYMHWARQAPGQGL |
| SEQ ID NO. 100 | EWMGIINPSGGSTSYAQKFQGRVTMTRDTSTSTVYMELSSLRSED |
| | TAVYYCARGKWWLYNWGKGTLVTVSS |
| MEDI-14-VL | QSVLTQPPSVSAAPGQKVTISCSGGSSNIGNSYVSWYQQLPGTAP |
| SEQ ID NO. 101 | KLLIYDNNKRPSGIPDRFSGSKSGTSATLAITGLQTGDEADYYCG |
| | TWDTSPPTKPLFGTGTKLTVL |
| MEDI-15-VH | QVQLVQSGAEVKKPGASVKVSCKASGYAFTSYYMHWARQAPGQGL |
| SEQ ID NO. 102 | EWMGIINPSGGSTSYAQKFQGRVTMTRDTSTSTVYMELSSLRSED |
| | TAVYYCARGKWWLYNWGKGTLVTVSS |
| MEDI-15-VL | QSVLTQPPSVSAAPGQKVTISCSGGSSNIGNSYVSWYQQLPGTAP |
| SEQ ID NO. 103 | KLLIYDNNKRPSGIPDRFSGSKSGTSATLAITGLQTGDEADYYCG |
| | TWDTSTHRHPLFGTGTKLTVL |
| MEDI-16-VH | QVQLVQSGAEVKKPGASVKVSCKASGYAFTSYYMHWARQAPGQGL |
| SEQ ID NO. 104 | EWMGIINPSGGSTSYAQKFQGRVTMTRDTSTSTVYMELSSLRSED |
| | TAVYYCARGKWWLYNWGKGTLVTVSS |
| MEDI-16-VL | QSVLTQPPSVSAAPGQKVTISCSGGSSNIGNSYVSWYQQLPGTAP |
| SEQ ID NO. 105 | KLLIYDNNKRPSGIPDRFSGSKSGTSATLAITGLQTGDEADYYCG |
| | TWDTSTTYHPIFGTGTKLTVL |
| MEDI-17-VH | QVQLVQSGAEVKKPGASVKVSCKASGYAFTSYYMHWARQAPGQGL |
| SEQ ID NO. 106 | EWMGIINPSGGSTSYAQKFQGRVTMTRDTSTSTVYMELSSLRSED |
| | TAVYYCARGKWWWQHWGKGTLVTVSS |
| MEDI-17-VL | QSVLTQPPSVSAAPGQKVTISCSGGSSNIGNSYVSWYQQLPGTAP |
| SEQ ID NO. 107 | KLLIYDNNKRPSGIPDRFSGSKSGTSATLAITGLQTGDEADYYCG |
| | TWDTSPVDRPIFGTGTKLTVL |
| MEDI-18-VH | QVQLVQSGAEVKKPGASVKVSCKASGYAFTSYYMHWARQAPGQGL |
| SEQ ID NO. 108 | EWMGIINPSGGSTSYAQKFQGRVTMTRDTSTSTVYMELSSLRSED |
| | TAVYYCARGKWWWQHWGKGTLVTVSS |
| MEDI-18-VL | QSVLTQPPSVSAAPGQKVTISCSGGSSNIGNSYVSWYQQLPGTAP |
| SEQ ID NO. 109 | KLLIYDNNKRPSGIPDRFSGSKSGTSATLAITGLQTGDEADYYCG |
| | TWDTSTTPMPVFGTGTKLTVL |
| MEDI-19-VH | QVQLVQSGAEVKKPGASVKVSCKASGYAFTSYYMHWARQAPGQGL |
| SEQ ID NO. 110 | EWMGIINPSGGSTSYAQKFQGRVTMTRDTSTSTVYMELSSLRSED |
| | TAVYYCARGKWWWQHWGKGTLVTVSS |
| MEDI-19-VL | QSVLTQPPSVSAAPGQKVTISCSGGSSNIGNSYVSWYQQLPGTAP |
| SEQ ID NO. 111 | KLLIYDNNKRPSGIPDRFSGSKSGTSATLAITGLQTGDEADYYCG |
| | TWDTSTTYHPIFGTGTKLTVL |
| MEDI-20-VH | QVQLVQSGAEVKKPGASVKVSCKASGYAFTSYYMHWARQAPGQGL |
| SEQ ID NO. 112 | EWMGIINPSGGSTSYAQKFQGRVTMTRDTSTSTVYMELSSLRSED |
| | TAVYYCARGKYWMYDWGKGTLVTVSS |
| MEDI-20-VL | QSVLTQPPSVSAAPGQKVTISCSGGSSNIGNSYVSWYQQLPGTAP |
| SEQ ID NO. 113 | KLLIYDNNKRPSGIPDRFSGSKSGTSATLAITGLQTGDEADYYCG |
| | TWDTSTVWEWPFGTGTKLTVL |
| MEDI-21-VH | QVQLVQSGAEVKKPGASVKVSCKASGYAFTSYYMHWARQAPGQGL |
| SEQ ID NO. 114 | EWMGIINPSGGSASYAQKFQGRVTMTRDTSTSTVYMELSSLRSED |
| | TAVYYCARGKYWMYDWGKGTLVTVSS |
| MEDI-21-VL | QSVLTQPPSVSAAPGQKVTISCSGGSSNIGNSYVSWYQQLPGTAP |
| SEQ ID NO. 115 | KLLIYDNNKRPSGIPDRFSGSKSGTSATLAITGLQTGDEAVYFCG |
| | TWDTSTVWEWPFGTGTKLTVL |
| MEDI-22-VH | QVQLVQSGAEVKKPGASVKVSCKASGYAFTSYYMHWARQAPGQGL |
| SEQ ID NO. 116 | EWMGIINPSGGSTSYAQKFQGRVTMTRDTSTSTVYMELSSLRSED |
| | TAVYYCARGKYWMYDWGKGTLVTVSS |
| MEDI-22-VL | QPVLTQPPSVSAAPGQKVTISCSGGSSNIGNSYVSWYQQLPGTAP |
| SEQ ID NO. 117 | KLLIYDNNKRPSGIPDRFSGSKSGTSATLAITGLQTGDEADYFCG |
| | TWDTSTVWEWPFGTGTKLTVL |
| MEDI-23-VH | QVQLVQSGAEVRKPGASVKVSCKASGYAFTSYYMHWARQAPGQGL |
| SEQ ID NO. 118 | EWMGIINPSGGSTSYAQKFQGRVTMTRDTSTSTVYMELSSLRSED |
| | TAVYYCARGKYWMYDWGKGTLVTVSS |
| MEDI-23-VL | QSVLTQPPSVSAAPGQKVTISCSGGSSNIGNNYVSWYQQLPGTAP |
| SEQ ID NO. 119 | KLLIYDNNKRPPGIPDRFSGSKSGTSATLAITGLQTGDEADYYCG |
| | TWDTSTVWEWPFGTGTKLTVL |
| MEDI-24-VH | QVQLVQSGAEVKKPGASVKVSCKASGYAFTSYYMHWARQAPGQGL |
| SEQ ID NO. 120 | EWMGIINPRGGSTSYAQKFQGRVTMTRDTSTSTVYMELSSLRSED |
| | TAVYYCARGKYWMYDWGKGTLVTVSS |
| MEDI-24-VL | QSVLTQPPSVSAAPGQKVTISCSGGSSNIGNSYVSWYQQLPGTAP |
| SEQ ID NO. 121 | KLLIYDNNKRPSGIPDRFSGSKSGTSATLAITGLQTGDEADYFCG |
| | TWDTSTVWEWPFGTGTKLTVL |
| MEDI-25-VH | QVQLVQSGAEVKKPGASVKVSCKASGYAFTSYYMHWARQAPGQGL |
| SEQ ID NO. 122 | EWMGIINPRGGSASYAQKFQGRVSMTRDTSTSTVYMELSSLRSED |
| | TAVYYCARGKYWMYDWGKGTLVTVSS |
| MEDI-25-VL | QSVLTQPPSVSAAPGQKVTISCSGGSSNIGNSYVSWYQQLPGTAP |
| SEQ ID NO. 123 | KLLIYDNNKRPSGIPDRFSGSKSGTTATLAITGLQTGDEADYYCG |
| | TWVTSTVWEWPFGTGTKLTVL |
| MEDI-26-VH | QVQLVQSGAEVKKPGASVKVSCKASGYAFTSYYMHWARQAPGQGL |
| SEQ ID NO. 124 | EWMGIINPSGGSTSYAQKFQGRVTMTRDTSTSTVYMELSSLRSED |
| | TAVYYCARGKYWMYDWGKGTLVTVSS |
| MEDI-26-VL | QSVLTQPPSVSAAPGQKVTISCSGGSSNIGNSYVSWYQQLPGTAP |
| SEQ ID NO. 125 | KLLIYDNNKRPSGIPDRFSGSKSGTSATLAITGLQTGDEADYFCG |
| | TWDTSTVWEWPFGTGTKLTVL |
| MEDI-27-VH | QVQLVQSGAEVRKPGASVKVSCKASGYAFTSYYMHWARQAPGQGL |
| SEQ ID NO. 126 | EWMGIINPSGGSTSYAQKFQGRVTMTRDTSTSTVYMELSSLRPED |
| | TAVYYCARGKYWMYDWGKGTQVTVSS |
| MEDI-27-VL | QSVLTQPPLVSAAPGQKVTISCSGGSSNIGNSYVSWYQRLPGTAP |
| SEQ ID NO. 127 | KLLIYDNNKRPSGIPDRFSGSKSGTSATLAITGLQTGDEADYYCG |
| | TWDTSTVWEWPFGTGTKLTVL |
| MEDI-28-VH | QVQLVQSGAEVKKPGASVKVSCKASGYAFTSYYMHWARQAPGQGL |
| SEQ ID NO. 128 | EWMGIINPSGGSTSYAQKFQGRVTMTRDTSTSTVYMELSSLRSED |
| | TAVYYCARGKYWMYDWGNGTLVTVSS |
| MEDI-28-VL | LPVLTQPPSVSAAPGQKVTISCSGGSSSIGNSYVSWYQQLPGAAP |
| SEQ ID NO. 129 | KLLIYDNNKRPSGIPDRFSGFRSGTSATLAITGLQTGDEADYYCG |
| | TWDTSPVWEWPFGTGTKLTVL |
| MEDI-29-VH | QVQLVQSGAEVKKPGASVKVSCKASGYAFTSYYMHWARQAPGQGL |
| SEQ ID NO. 130 | EWMGIINPSGGSTSYAQKFQGRVTMTRDTSTSTVYMELSSLRSED |
| | TAVYYCARGKYWMYDWGKGTRVTVSS |
| MEDI-29-VL | QSVLTQPPSVSAAPGQKVTISCSGGSSNIGNSYVSWYQQLPGTAP |
| SEQ ID NO. 131 | KLLIYDNNKRPSGIPDRFSGSKSGTSATLAITGLQTGDEADYYCG |
| | TWDTSPVWEWPFGTGTKLTVL |
| MEDI-30-VH | QVQLVQSGAEVKKPGASVKVSCKASGYAFTSYYMHWARQAPGQGL |
| SEQ ID NO. 132 | EWMGIINPSGGSTSYAQKFQGRVTMTRDTSTSTVYMELSSLRSED |
| | TAVYYCARGKYWMYDWGKGTLVTVSS |
| MEDI-30-VL | QSVLTQPPSVSAAPGQKVTISCSGGSSNIGNSYVSWYQRLPGAAP |
| SEQ ID NO. 133 | KLLIYDNNKRPSGIPDRFSGSKSGTSATLAITGLQTGDEADYYCG |
| | TWDTSTVWEWPFGTGTKLTVL |
| MEDI-31-VH | QVQLVQSGAEVKKPGASVKVSCKASGYAFTSYYMHWARQAPGQGL |
| SEQ ID NO. 134 | EWMGIINPSGGSTSYAQKFQGRVTMTRDTSTSTVYMELSSLRSED |
| | TAVYYCARGKYWMYDWGKGTLVTVSS |
| MEDI-31-VL | QSVLTQPPSVSAAPGQKVTISCSGGSSSIGNSYVSWYQQLPGTAP |
| SEQ ID NO. 135 | KLLIYDNNKRPSGIPDRFSGSKSGTSATLAITGLQTGDEADYYCG |
| | TWATSPVWEWPFGTGTKLTVL |
| MEDI-32-VH | QVQLVQSGAEVKKPGASVKVSCKASGYAFTSYYMHWARQAPGQGL |
| SEQ ID NO. 136 | EWMGIINPSGGSTSYAQKFQGRVTMTRDTSTSTVYMELSSLRSED |
| | TAVYYCARGKYWMYDWGKGTLVTVSS |
| MEDI-32-VL | QSVLTQPPSVSAAPGQKVTISCSGGSSNIGNSYVSWYQQLPGTAP |
| SEQ ID NO. 137 | KLLIYDNNKRPSGIPDRFSGSKSGTSATLAITGLQTGDEADYFCG |
| | TWDTSTAWEWPFGTGTKLTVL |
| MEDI-33-VH | QVQLVQSGAEEKKPGASVKVSCKASGYAFTSYYMHWARQAPGQGL |
| SEQ ID NO. 138 | EWMGIINPSGGSTSYAQKFQGRVTMTRDTSTSTVYMELSSLRSED |
| | TAVYYCARGKYWMYDWGKGTLVTVSS |
| MEDI-33-VL | QSALTQPPSVSAAPGQKVTISCSGGSSNIGNSYVSWYQQLPGTAP |
| SEQ ID NO. 139 | KLLIYDNNKRPSGIPDRFSGSKSGTSATLAITGLQTGDEADYFCG |
| | TWDTSTVWEWPFGTGTKLTVL |
| MEDI-34-VH | QVQLVQSGAEVKKPGASVKVSCKASGYAFTSYYMHWARQAPGQGL |
| SEQ ID NO. 140 | EWMGIINPSGGSTSYAQKFQGRVSMTRDTSTSTVYMELSSLRSED |
| | TAVYYCARGKYWMYDWGKGTLVTVSS |
| MEDI-34-VL | QSVLTQPPSVSAAPGQKVTISCSGGSSNIGNSYVSWYQQLPGTAP |
| SEQ ID NO. 141 | KLLIYDNNKRPSGIPDRFSGSKSGTSATLAITGLQTGDEADYFCG |
| | TWDTSTVWEWPFGTGTKLTVL |
| MEDI-35-VH | QVQLVQSGAEVKKPGASVKVSCKASGYAFTSYYMHWARQAPGQGL |
| SEQ ID NO. 142 | EWMGIINPSGGSTSYAQKFQGRVTMTRDTSTSTVYMELSSLRSED |
| | TAVYYCARGKYWMYDWGKGTLVTVSS |
| MEDI-35-VL | QSVLTQPPSVSAAPGQKVTISCSGGSSNIGNSYVSWYQQLPGTAP |
| SEQ ID NO. 143 | KLLIYDNNKRPSGIPDRFSGSKSGTSATLAITGLQTGDEADYYCG |
| | TWDTSPVWEWPFGTGTKLTVL |
| MEDI-36-VH | QVQLVQSGAEVKKPGASVKVSCKASGYAFTSYYMHWARQAPGQGL |
| SEQ ID NO. 144 | EWMGIINPSGGSASYAQKFQGRVTMTRDTSTSTVYMELSSLRSED |
| | TAVYYCARGKYWMYDWGKGTLVTVSS |
| MEDI-36-VL | QSVLTQPPSVSAAPGQKVTISCSGGSSNIGNSYVSWYQQLPGTAP |
| SEQ ID NO. 145 | KLLIYDNNKRPSGIPDRFSGSKSGTSATLAITGLQTGDEADYYCG |
| | TWDSSTVWEWPFGTGTKLTVL |
| MEDI-37-VH | QVQLVQSGAEVKKPGASVKVSCKASGYAFTSYYMHWARQAPGQGL |
| SEQ ID NO. 146 | EWMGIINPRGGSTSYAQKFQGRVAMTRDTSTSTVYMELSSLRPED |
| | TAVYYCARGKYWMYDWGKGTLVTVSS |
| MEDI-37-VL | QSVLTQPPSVSAAPGQKVTISCSGGGSSIGNSYVSWYQQLPGTAP |
| SEQ ID NO. 147 | KLLIYDNNKRPSGVPDRFSGSKSGTSATLAITGLQTGDEADYYCG |
| | TWDTSPVWEWPFGTGTKLTVL |
| MEDI-38-VH | QVQLVQSGAEVKKPGASVKVSCKASGYAFTSYYMHWARQAPGQGL |
| SEQ ID NO. 148 | EWMGIINPSGGSASYAQKFQGRVTMTRDTSTSTVYMELSSLRSED |
| | TAVYYCARGKYWMYDWGKGTLVTVSS |
| MEDI-38-VL | QSVLTQPPSVSAAPGQKVTISCSGGSSNIGNSYVSWYQQLPGTAP |
| SEQ ID NO. 149 | KLLIYDNNKRPSGIPDRFSGSKSGTSATLAITGLQTGDEADYFCG |
| | TWDTSTVWEWPFGTGTKLTVL |
| MEDI-39-VH | QVQLVQSGAEVKKPGASVKVSCKASGYAFTSYYMHWARQAPGQGL |
| SEQ ID NO. 150 | EWMGIINPRGGSTSYAQKFQGRVTMTRDTSTSTVYMELSSLRSED |
| | TAVYYCARGKYWMYDWGKGTLVTVSS |
| MEDI-39-VL | QSVLTQPPSVSAAPGQKVTISCSGGSSNIGNSYVSWYQQLPGTAP |
| SEQ ID NO. 151 | KLLIYDNNKRPSGIPDRFSGSKSGTSATLAITGLQTGDEADYYCG |
| | TWDTSTAWEWPFGTGTKLTVL |
| MEDI-40-VH | QVQLVQSGAEVKKPGASVKVSCKASGYAFTSYYMHWARQAPGQGL |
| SEQ ID NO. 152 | EWMGIINPSGGSTSYAQKFQGRVTMTRDTSTSTVYMELSSLRSED |
| | TAVYYCARGKYWMYDWGKGTLVTVSS |
| MEDI-40-VL | QSVLTQPPSVSAAPGQKVTISCSGGSSNIGNSYVSWYQQLPGTAP |
| SEQ ID NO. 153 | KLLIYDNNKRPSGIPDRFSGSKSGTSATLAITGLQTGDEADYYCG |
| | TWDSSTVWEWPFGTGTKLTVL |
| MEDI-41-VH | QVQLVQSGAEVRKPGASVKVSCKASGYAFTSYYMHWARQAPGQGL |
| SEQ ID NO. 154 | EWMGIINPSGGSTSYAQKFQGRVTMTRDTSTSTVYMELSSLRPED |
| | TAVYYCARGKYWMYDWGKGTLVTVSG |
| MEDI-41-VL | QSVLTQPPSVSAAPGQKVTISCSGGSTNIGNSYVSWYQRLPGTAP |
| SEQ ID NO. 155 | KLLIYDNNKRPPGIPDRFSGSKSGTSATLAITGLQTGDEADYYCG |
| | TWDTSTVWEWPFGTGTKLTVL |
| MEDI-42-VH | QVQLVQSGAEVKKPGASVKVSCKASGYAFTSYYMHWARQAPGQGL |
| SEQ ID NO. 156 | EWVGIINPSGGSTSYAQKFQGRVTMTRDTSTSTVYMELSSLRSGD |
| | TAVYYCARGKYWMYDWGKGTLVTVSS |
| MEDI-42-VL | QAVLTQPPSVSAAPGQKVTISCSGGSSNIGNSYVSWYQRLPGAAP |
| SEQ ID NO. 157 | KLLIYDNNKRPSGIPDRFSGSKSGTSATLAITGLQTGDEADYYCG |
| | TWDTSTGWEWPFGTGTKLTVL |
| MEDI-37GL- | QVQLVQSGAEVKKPGASVKVSCKASGYAFTSYYMHWVRQAPGQGL |
| VH SEQ ID | EWMGIINPRGGSTSYAQKFQGRVTMTRDTSTSTVYMELSSLRSED |
| NO. 158 | TAVYYCARGKYWMYDWGKGTLVTVSS |
| MEDI-37GL- | QSVLTQPPSVSAAPGQKVTISCSGGGSSIGNSYVSWYQQLPGTAP |
| VL SEQ ID | KLLIYDNNKRPSGIPDRFSGSKSGTSATLGITGLQTGDEADYYCG |
| NO. 159 | TWDTSPVWEWPFGTGTKLTVL |
| MEDI-37GL- | SYYMH |
| HCDR1SEQ | |
| ID NO. 160 | |
| MEDI-37GL- | IINPRGGSTSYAQKFQG |
| HCDR2 SEQ | |
| ID NO. 161 | |
| MEDI-37GL- | GKYWMYD |
| HCDR3 SEQ | |
| ID NO. 162 | |
| MEDI-37GL- | SGGGSSIGNSYVS |
| LCDR1 SEQ ID | |
| NO. 163 | |
| MEDI-37GL- | DNNKRPS |
| LCDR2 SEQ ID | |
| NO. 164 | |
| MEDI-37GL- | GTWDTSPVWEWP |
| LCDR3 SEQ ID | |
| NO. 165 | |

In certain embodiments, an antibody or antigen-binding fragment thereof of the disclosure comprises a LCVR/HCVR sequence pair of AJOU-90-VL/AJOU-83-VH.

In certain embodiments, an antibody or antigen-binding fragment thereof of the disclosure comprises an HCVR comprising an HCDR1 sequence of AJOU-84-HCDR1, an CHDR2 sequence of AJOU-85-HCDR2, and an HCDR3 sequence of AJOU-32-HCDR3, and an LCVR comprising an LCDR1 of AJOU-96-LCDR1, and LCDR2 of AJOU-60-LCDR2, and an LCDR3 of AJOU-68-LCDR3.

The antibodies recited below in Table 3 are described in more detail in WO2020/096381 and Kim et al. (Scientific Reports. 9: 7772. 2019), incorporated herein by reference in their entireties for all purposes.

TABLE 3

| Sequence ID | Sequence |
|---|---|
| AJOU-1-VH | EVQLLESGGGLVQPGGSLRLSCAVSGFTFSNYAMSWVRQAPGKGL |
| SEQ ID NO. 166 | EWVSAISSGGGNIYYADSVKGRFTISRDNSKNTLYLQMNSLRAED |
| | TAVYYCAKLRRYFDYWGQGTLVTVSS |
| AJOU-2-VH | EVQLLESGGGLVQPGGSLRLSCAASGFTFSDYAMSWVRQAPGKGL |
| SEQ ID NO. 167 | EWVSAISSGGSSIYYADSVKGRFTISRDNSKNTLHLQMNSLRAED |
| | TAVYYCARGPQRSATAVFDYWGQGTLVTVSS |
| AJOU-3-VH | EVQLLESGGGLVQPGGSLRLSCAASGFTFSNYAMSWVRQAPGKGL |
| SEQ ID NO. 168 | EWVSWISPNSGNIYYADSVKGRFTISRDNSKNTLYLQMNSLRAED |
| | TAVYYCARRPLSAAWSHSSYYNAMDVWGQGTLVTVSS |
| AJOU-4-VH | EVQLLESGGGLVQPGGSLRLSCAASGFTFSGYAMSWVRQAPGKGL |
| SEQ ID NO. 169 | EWVSLISHSGSNTYYADSVKGRFTISRDNSKNTLYLQMNSLRAED |
| | TAVYYCARPHRAFDYWGQGTLVTVSS |
| AJOU-5-VH | EVQLLESGGGLVQPGGSLRLSCAASGFTFSNYAMSWVRQAPGKGL |
| SEQ ID NO. 170 | EWVSGISHGSGSIYYADSVKGRFTISRDNSKNTLYLQMNSLRAED |
| | TAVYYCARPHRAFDYWGQGTLVTVSS |
| AJOU-6-VH | EVQLLESGGGLVQPGGSLRLSCAASGFTFSNYAMSWVRQAPGKGL |
| SEQ ID NO. 171 | EWVSGISHGNGSIYYADSVKGRFTISRDNSKNTLYLQMNSLRAED |
| | TAVYYCAKTGRHFDYWGQGTLVTVSS |
| AJOU-7-VH | EVQLLESGGGLVQPGGSLRLSCAASGFTFSNYAMSWVRQAPGKGL |
| SEQ ID NO. 172 | EWVSSISPSGSSIYYADSVKGRFTISRDNSKNTLYLQMNSLRAED |
| | TAVYYCARSYRAFDYWGQGTLVTVSS |
| AJOU-8-VH | EVQLLESGGGLVQPGGSLRLSCAASGFTFSNYAMSWVRQAPGKGL |
| SEQ ID NO. 173 | EWVSAISPSGGSIYYADSVKGRFTISRDNSKNTLYLQMNSLRAED |
| | TAVYYCARAKRAFDYWGQGTLVTVSS |
| AJOU-9-VH | EVQLLESGGGLVQPGGSLRLSCAASGFTFSNYAMSWVRQAPGKGL |
| SEQ ID NO. 174 | EWVSAISPGSGSTYYADSVKGRFTISRDNSKNTLYLQMNSLRAED |
| | TAVYYCAKFRRHFDYWGQGTLVTVSS |
| AJOU-10-VH | EVQLLESGGGLVQPGGSLRLSCAASGFTFSNYAMSWVRQAPGKGL |
| SEQ ID NO. 175 | EWVSAISSGGGNIYYADSVKGRFTISRDNSKNTLYLQMNSLRAED |
| | TAVYYCARVHRAFDYWGQGTLVTVSS |
| AJOU-69-VH | EVQLLESGGGLVQPGGSLRLSCAASGFTFSNYAMSWVRQAPGKGL |
| SEQ ID NO. 176 | EWVSAITSSGRSIYYADSVKGRFTISRDNSKNTLYLQMNSLRAED |
| | TAVYYCARVHRAFDYWGQGTLVTVSS |
| AJOU-70-VH | EVQLLESGGGLVQPGGSLRLSCAASGFTFSNYAMSWVRQAPGKGL |
| SEQ ID NO. 177 | EWVSAITSSGANIYYADSVKGRFTISRDNSKNTLYLQMNSLRAED |
| | TAVYYCARVHRAFDYWGQGTLVTVSS |
| AJOU-71-VH | EVQLLESGGGLVQPGGSLRLSCAASGFTFSNYAMSWVRQAPGKGL |
| SEQ ID NO. 178 | EWVSAITSSGGNIYYADSVKGRFTISRDNSKNTLYLQMNSLRAED |
| | TAVYYCARVHRAFDYWGQGTLVTVSS |
| AJOU-72-VH | EVQLLESGGGLVQPGGSLRLSCAASGFTFSNYAMSWVRQAPGKGL |
| SEQ ID NO. 179 | EWVSAITAGGGSIYYADSVKGRFTISRDNSKNTLYLQMNSLRAED |
| | TAVYYCARVHRAFDYWGQGTLVTVSS |
| AJOU-83-VH | EVQLLESGGGLVQPGGSLRLSCAASGFTFSRHAMAWVRQAPGKGL |
| SEQ ID NO. 180 | EWVSAITSSGRSIYYADSVKGRFTISRDNSKNTLYLQMNSLRAED |
| | TAVYYCARVHRAFDYWGQGTLVTVSS |
| AJOU-33-VL | QSVLTQPPSASGTPGQRVTISCSGSSSNIGNNYVNWYQQLPGTAP |
| SEQ ID NO. 181 | KLLIYDNSHRPSGVPDRFSGSKSGTSASLAISGLRSEDEADYYCG |
| | TWDASLSAYVFGGGTKLTVL |
| AJOU-34-VL | QSVLTQPPSASGTPGQRVTISCSGSSSNIGNNNVSWYQQLPGTAP |
| SEQ ID NO. 182 | KLLIYANSKRPSGVPDRFSGSKSGTSASLAISGLRSEDEADYYCG |
| | SWDDSLSAYVFGGGTKLTVL |
| AJOU-35-VL | QSVLTQPPSAPGTPGQRVTISCTGSSSNIGSNSVNWYQQLPGTAP |
| SEQ ID NO. 183 | KLLIYDDSHRPSGVPDRFSGSKSGTSASLAISGLRSEDEADYYCD |
| | AWDSSLSAYVFGGGTKLTVL |
| AJOU-36-VL | QSVLTQPPSASGTPGQRVTLSCTGSSSNIGSNYVSWYQQLPGTAP |
| SEQ ID NO. 184 | KLLIYADSQRPSGVPDRFSGSKSGTSASLAISGLRSEDEADYYCG |
| | TWDDSLSGYVFGGGTKLTVL |
| AJOU-37-VL | QSVLTQPPSASGTPGQRVTISCSSSSSNIGSNYVSWYQQLPGTAP |
| SEQ ID NO. 185 | KLLIYSDSHRPSGVPDRFSGSKSGTSASLAISGLRSEDEADYYCG |
| | SWDYSLSAYVFGGGTKLTVL |
| AJOU-38-VL | QSVLTQPPSASGTPGQRVTISCTGSSSNIGNNTVSWYQQLPGTAP |
| SEQ ID NO. 186 | KLLIYDNSHRPSGVPDRFSGSKSGTSASLAISGLQSEDEADYYCG |
| | SWDYSLSAYVFGGGTKLTVL |
| AJOU-39-VL | QSVLTQPPSASGTPGQRVTISCTGSSSNIGNNDVNWYQQLPGTAP |
| SEQ ID NO. 187 | KLLIYYDSQRPSGVPDRFSGSKSGTSASLAISGLRSEDEADYYCA |
| | TWDASLSAYVFGGGTKLTVL |
| AJOU-40-VL | QSVLTQPPSASGTPGQRVTISCSGSSSNIGSNAVNWYQQLPGTAP |
| SEQ ID NO. 188 | KLLIYYDNQRPSGVPDRFSGSKSGTSASLAISGLRSEDEADYYCG |
| | TWDDSLNGYVFGGGTKLTVL |
| AJOU-41-VL | QSVLTQPPSASGTPGQRVTISCSGSSSNIGNNAVTWYQQLPGTAP |
| SEQ ID NO. 189 | KLLIYDDSHRPSGVPDRFSGSKSGTSASLAISGLRSEDEADYYCG |
| | SWDYSLSAYVFGGGTKLTVL |
| AJOU-42-VL | QSVLTQPPSASGTPGQRVTISCSGSSSNIGSNTFNWYQQLPGTAP |
| SEQ ID NO. 190 | KLLIYADSHRPSGVPDRFSGSKSGTSASLAISGLRSEDEADYYCG |
| | TWDYSLSGYVLGGGTKLTVL |
| AJOU-77-VL | QSVLTQPPSASGTPGQRVTISCSGSSSNIGSNTFNWYQQLPGTAP |
| SEQ ID NO. 191 | KLLIYADSHRPSGVPDRFSGSKSGTSASLAISGLRSEDEADYYCG |
| | TWDYSLSGYVLGGGTKLTVL |
| AJOU-78-VL | QSVLTQPPSASGTPGQRVTISCSGSSSNIGSNTFNWYQQLPGTAP |
| SEQ ID NO. 192 | KLLIYADSHRPSGVPDRFSGSKSGTSASLAISGLRSEDEADYYCG |
| | TWDYSLRGYVLGGGTKLTVL |
| AJOU-79-VL | QSVLTQPPSASGTPGQRVTISCSGSSSNIGSNTFNWYQQLPGTAP |
| SEQ ID NO. 193 | KLLIYADSHRPSGVPDRFSGSKSGTSASLAISGLRSEDEADYYCG |
| | YWDYSLSGYVLGGGTKLTVL |
| AJOU-80-VL | QSVLTQPPSASGTPGQRVTISCSGSSSNIGSNTFNWYQQLPGTAP |
| SEQ ID NO. 194 | KLLIYADSHRPSGVPDRFSGSKSGTSASLAISGLRSEDEADYYCG |
| | TWDYSLSGYVLGGGTKLTVL |
| AJOU-86-VL | QSVLTQPPSASGTPGQRVTISCSGSSANSRTDGFNWYQQLPGTAP |
| SEQ ID NO. 195 | KLLIYADSHRPSGVPDRFSGSKSGTSASLAISGLRSEDEADYYCG |
| | TWDYSLSGYVLGGGTKLTVLG |
| AJOU-87-VL | QSVLTQPPSASGTPGQRVTISCSGSAQFGSRDNFNWYQQLPGTAP |
| SEQ ID NO. 196 | KLLIYADSHRPSGVPDRFSGSKSGTSASLAISGLRSEDEADYYCG |
| | TWDYSLSGYVLGGGTKLTVLG |
| AJOU-88-VL | QSVLTQPPSASGTPGQRVTISCSGSTKQMHNYQFNWYQQLPGTAP |
| SEQ ID NO. 197 | KLLIYADSHRPSGVPDRFSGSKSGTSASLAISGLRSEDEADYYCG |
| | TWDYSLSGYVLGGGTKLTVLG |
| AJOU-89-VL | QSVLTQPPSASGTPGQRVTISCSGSLLRGENLQFNWYQQLPGTAP |
| SEQ ID NO. 198 | KLLIYADSHRPSGVPDRFSGSKSGTSASLAISGLRSEDEADYYCG |
| | TWDYSLSGYVLGGGTKLTVLG |
| AJOU-90-VL | QSVLTQPPSASGTPGQRVTISCSGSPLFPDSGSFNWYQQLPGTAP |
| SEQ ID NO. 199 | KLLIYADSHRPSGVPDRFSGSKSGTSASLAISGLRSEDEADYYCG |
| | TWDYSLSGYVLGGGTKLTVLG |
| AJOU-91-VL | QSVLTQPPSASGTPGQRVTISCSGSAALDLSPSFNWYQQLPGTAP |
| SEQ ID NO. 200 | KLLIYADSHRPSGVPDRFSGSKSGTSASLAISGLRSEDEADYYCG |
| | TWDYSLSGYVLGGGTKLTVLG |
| AJOU-84- | RHAMA |
| HCDR1 SEQ | |
| ID NO. 201 | |
| AJOU-85- | AITSSGRSIYYADSVKG |
| HCDR2 SEQ ID | |
| NO. 202 | |
| AJOU-32- | VHRAFDY |
| HCDR3 SEQ ID | |
| NO. 203 | |
| AJOU-96- | SGSPLFPDSGSFN |
| LCDR1 SEQ ID | |
| NO. 204 | |
| AJOU-60- | ADSHRPS |
| LCDR2 SEQ ID | |
| NO. 205 | |
| AJOU-68- | GTWDYSLSGYV |
| LCDR3 SEQ ID | |
| NO. 206 | |

In certain embodiments, an antibody or antigen-binding fragment thereof of the disclosure comprises light chain variable region (LCVR) and heavy chain variable region (HCVR) sequence pairs (LCVR/HCVR) selected from the group consisting of 11/3, 27/19, 43/35, 59/51, 75/67, 91/83, 107/99, 123/115, 155/147, and 171/163.

The antibodies recited below in Table 4 are described in more detail in U.S. Pat. Nos. 7,605,237 and 7,608,693, incorporated herein by reference in their entireties for all purposes.

TABLE 4

| Sequence ID | Sequence |
|---|---|
| REGN-VH-3 | QVQLVQSGAEVKKPGASVKVSCKASGYTFTNYGISWVRQAPGQG |
| SEQ ID NO. 207 | LEWMGWISVYNGKTNYAQKLQGRVTMTTDTSTTTAYMEMRSLRS |
| | DDTAVYYCARGSGYDLDYWGQGTLVSVSS |
| REGN-VH-19 | EVQLVESGGGLVQPGGSLRLSCAASGFTFSSFWMTWVRQAPGKG |
| SEQ ID NO. 208 | LEWVANIKQDGSEKYYVDSVKGRFTISRDNAKNSLYLQMNSLRA |
| | EDTAVYYCARDPGRTMVRGGIRYYYGMDVWGQGTTVTVSS |
| REGN-VH-35 | EVKLAESGGGLVQPGGSLRLSCAASGFTFSSHWMNWVRQAPGKG |
| SEQ ID NO. 209 | LEWVANIKQDGSDKYYVDSVKGRFTISRDNAKNSLYLQLNSLIA |
| | EDTAVYYCARDRGVRPPRGAFDIWGQGTMVTVSS |
| REGN-VH-51 | QVQLVQSGAEVKKPGASVKVSCKASGYTFNSYGISWVRQAPGQG |
| SEQ ID NO. 210 | LEWMGWIRTYNGNTNYAQKLQGRVTMTTDTSTSTAYMELRSLRS |
| | DDTAVYYCARDEARIVVAGTTPYYYGMDVWGQGTTVTVSS |
| REGN-VH-67 | QVQLVESGGGLVQPGGSLRLSCAVSGFTISDHYMSWIRQAPGKG |
| SEQ ID NO. 211 | LEWISYISSSGSKIYYADSVKGRFTISRDNAKNSLFLQMNSLRA |
| | EDTAVYYCARTRQLVGDYWGQGTLVTVSS |
| REGN-VH-83 | EVQLVESGGGLVQPGRSLRLSCAASGFTFDNYAMHWVRQAPGKG |
| SEQ ID NO. 212 | LEWVSGIRWNSGSIGYADSVKGRFTISRDNAKNSLYLQMNSLRA |
| | EDTALYYCAKEGGYSGYRPGPFFDYWGQGTLVTVSS |
| REGN-VH-99 | QVQLVQSGAEVKKPGASVKVSCKASGYTFTNYGISWVRQAPGQG |
| SEQ ID NO. 213 | LEWMGWISVYNGHTNYAQKLQGRVTMTTDTSTSTAYMELRSLRS |
| | DDTAVYYCARGSGYDFDSWGQGTLVTVSS |
| REGN-VH-115 | QVQLVQSGAEVKKPGASVKVSCKASRYTFTSYDINWVRQATGQG |
| SEQ ID NO. 214 | LEWMGWMNPNSGNTGYAQKFQGRVTMTRNTSTSTAYMELSSLRS |
| | EDTAVYYCARVRRFFDYWGQGTLVTVSS |
| REGN-VH-147 | QVQLVQSGPEVKKPGASVKVSCKASGYTFTNYGISWVRQAPGQG |
| SEQ ID NO. 215 | LEWMGWISVYNGNINYAQKLQGRVTMTTDTSTSTAYMDLRSLRS |
| | DDTAVYYCARGSGYDFDYWGQGTLVTVSS |
| REGN-VH-163 | QVQLVQSGAEVKKPGASVKVSCKDSAYTFNRYGISWVRQAPGQG |
| SEQ ID NO. 216 | LEWMGWISAYTGNTVYAQKLQGRVTMTTDNSTSTAYMELRSLRS |
| | DDTAVYYCARDKSIFGVVRGFDYWGQGTLVTVSS |
| REGN-VL-11 | AIQMTQSPSSLSASVGDRVTITCRASQGIRNALGWYQQKPGKAP |
| SEQ ID NO. 217 | KLLIYAASSLQSGVPSRFSGSGSGTDFTLTFSSLQPEDFATYYC |
| | LQDFNYPYTFGQGTKLEIK |
| REGN-VL-27 | DIQMTQSPSSVSASVGDRVTISCRASQGVSSWLAWYQQKPGNAP |
| SEQ ID NO. 218 | KLLISAASSIQSGVPSRFSGSGSGTDFTLTISSLQPEDFATYYC |
| | QQANSFPLTFGGGTKVEIK |
| REGN-VL-43 | DIQMTQSPSSVSASVGDRVTITCRASQGISSWLAWYQQKPGKAP |
| SEQ ID NO. 219 | KLLIYAASSFQSGVPSRFSGSGSGTDFTLTISSLQPEDFATYFC |
| | QQANSFPLTFGGGTTVEIK |
| REGN-VL-59 | DIQMTQSPSSVSASVGDRVTITCRASQDISIWLAWYQQSPGKAP |
| SEQ ID NO. 220 | KLLINVASRLQSGVPSRFSGSGSGTDFTLTINSLQPEDFVTYYC |
| | QQANSFPITFGQGTRLATK |
| REGN-VL-75 | DIQLTQSPSFLSASVGDRVTITCWASQGISSYLAWYQQKPGKAP |
| SEQ ID NO. 221 | KLLIFAASTLQSGVPSRFSGSGSGTEFTLTISSLQPEDFATYYC |
| | QQLNSYPLTFGGGTKVEIR |
| REGN-VL-91 | EIVMTQSPATLSVSPGERATLSCRASQSVNYNLAWYQHKPGQAP |
| SEQ ID NO. 222 | RLLIYGASTRATGIPARFSGSGSGTEFTLTISSLQSEDFAVYYC |
| | QQYNNWPLTFGGGTKVEIK |
| REGN-VL-107 | AIQMTQSSSSLSASVGDRVTITCRASQAIRNALGWYQQKPGKAP |
| SEQ ID NO. 223 | KVLIYAASSLQSGIPSRFSGSGSGTDFTLTISSLQPEDFATYYC |
| | LQDYDYPYTFGQGTKLEIK |
| REGN-VL-123 | DIQLTQSPSFLSASVGDRVTITCWASQGIISYLAWYQQKPGKAP |
| SEQ ID NO. 224 | KLLIYAASTLHSGVPSRFSGSGSGTEFTLTISSLQPEDFATYYC |
| | HQLKSYPITFGQGTRLEIK |
| REGN-VL-155 | AIQMTQSPSSLSASVGDRVTITCRASQDIRNALGWYQQKPGKAP |
| SEQ ID NO. 225 | KLLIYAASSLQSGVPSRFSGSASGTDFTLTISSLQPEDFAAYYC |
| | LQDYNYPYTFGQGTKLEIK |
| REGN-VL-171 | EIVMTQSPVTLSLSPGERATLPCRASQSVSSSLAWYQQKAGQSP |
| SEQ ID NO. 226 | RLLIYGASTRATGIPARFSGSGSGTEFTLTISNLQSEDFAVYYC |
| | QQYNNWPLTFGGGTKVEIK |

The antibodies recited below in Table 5 are described in more detail in WO2022/052974, incorporated herein by reference in its entirety for all purposes.

TABLE 5

| EVQLLESGGGLVQPGGSLRLSCAASGFTLSSYAMHWVR | STSA-C27- |
|---|---|
| QAPGKGLEYVSGISSNGGSTYYANSVKGRFTISRDNPK | VH SEQ ID |
| NTLFLQMSSLRAEDTAVYYCVRVKVGYRGGMDVWGQGT | NO. 227 |
| TVTVSS | |
| EVQLLESGGGLVQPGGSLRLSCAASGFTLSSYAMHWVR | STSA-C27- |
| QAPGKGLEYVSGISPSGSSTYYANSVKGRFTISRDNPK | 6-33-VH |
| NTLFLQMSSLRAEDTAVYYCVRSKVRYRGGMDVWGQGT | SEQ ID NO. |
| TVTVSS | 228 |
| EVQLLESGGGLVQPGGSLRLSCAASGFTLSSYAMHWVR | STSA-C27- |
| QAPGKGLEYVSGISPSGVSTYYANSVKGRFTISRDNPK | 7-33-VH9 |
| NTLFLQMSSLRAEDTAVYYCVRVKVKYRGGMDVWGQGT | SEQ ID NO. |
| TVTVSS | 22 |
| EVQLLESGGGLVQPGGSLRLSCAASGFTLSSYAMHWVR | STSA-C27- |
| QAPGKGLEYVSGISPTSGSTYYANSVKGRFTISRDNPK | 24-56-VH |
| NTLFLQMSSLRAEDTAVYYCVRVKVRYRGGMDVWGQGT | SEQ ID NO. |
| TVTVSS | 230 |
| EVQLLESGGGLVQPGGSLRLSCAASGFTLSSYAMHWVR | STSA-C27- |
| QAPGKGLEYVSGISPTGTSTYYANSVKGRFTISRDNPK | 47-56-VH |
| NTLFLQMSSLRAEDTAVYYCVRVKGAYRGGMDVWGQGT | SEQ ID NO. |
| TVTVSS | 231 |
| EVQLLESGGGLVQPGGSLRLSCAASGFTLSSYAMHWVR | STSA-C27- |
| QAPGKGLEYVSGISSSGSSTYYANSVKGRFTISRDNPK | 33-33-VH |
| NTLFLQMSSLRAEDTAVYYCVRVKVAYRGGMDVWGQGT | SEQ ID NO. |
| TVTVSS | 232 |
| EVQLLESGGGLVQPGGSLRLSCAASGFTLSSYAMHWVR | STSA-C27- |
| QAPGKGLEYVSGISPSSTSTYYANSVKGRFTISRDNPK | 56-56-VH |
| NTLFLQMSSLRAEDTAVYYCVRVKVLYRGGMDVWGQGT | SEQ ID NO. |
| TVTVSS | 233 |
| EVQLLESGGGLVQPGGSLRLSCAASGFTLSSYAMHWVR | STSA-C27- |
| QAPGKGLEYVSGISPSSASTYYANSVKGRFTISRDNPK | 78-78-VH |
| NTLFLQMSSLRAEDTAVYYCVRVKSKYRGGMDVWGQGT | SEQ ID NO. |
| TVTVSS | 234 |
| EVQLLESGGGLVQPGGSLRLSCAASGFTLSSYAMHWVR | STSA-C27- |
| QAPGKGLEYVSGISGNSASTYYANSVKGRFTISRDNPK | 82-58-VH |
| NTLFLQMSSLRAEDTAVYYCVRVKLKYRGGMDVWGQGT | SEQ ID NO. |
| TVTVSS | 235 |
| EVQLLESGGGLVQPGGSLRLSCAASGFTLSSYAMHWVR | STSA-C27- |
| QAPGKGLEYVSGISHSGTSTYYANSVKGRFTISRDNPK | 54-54-VH |
| NTLFLQMSSLRAEDTAVYYCVRVRVLYRGGMDVWGQGT | SEQ ID NO. |
| TVTVSS | 236 |
| EVQLLESGGGLVQPGGSLRLSCAASGFTLSSYAMHWVR | STSA-C27- |
| QAPGKGLEYVSGISPSGVSTYYANSVKGRFTISRDNPK | 36-36-VH |
| NTLFLQMSSLRAEDTAVYYCVRVKVKYRGGMDVWGQGT | SEQ ID NO. |
| TVTVSS | 237 |
| EVQLLESGGGLVQPGGSLRLSCAASGFTLSSYAMHWVR | STSA-C27- |
| QAPGKGLEYVSGISSNGGSTYYANSVKGRFTISRDNPK | 53-53-VH |
| NTLFLQMSSLRAEDTAVYYCVRVFVRYRGGMDVWGQGT | SEQ ID NO. |
| TVTVSS | 238 |
| EVQLLESGGGLVQPGGSLRLSCAASGFTLSSYAMHWVR | STSA-C27- |
| QAPGKGLEYVSGISPTSASTYYANSVKGRFTISRDNPK | 67-67-VH |
| NTLFLQMSSLRAEDTAVYYCVRVKGRYRGGMDVWGQGT | SEQ ID NO. |
| TVTVSS | 239 |
| EVQLLESGGGLVQPGGSLRLSCAASGFTLSSYAMHWVR | STSA-C27- |
| QAPGKGLEYVSGISPTGGSTYYANSVKGRFTISRDNPK | 55-55-VH |
| NTLFLQMSSLRAEDTAVYYCVRVKGRYRGGMDVWGQGT | SEQ ID NO. |
| TVTVSS | 240 |
| EVQLLESGGGLVQPGGSLRLSCAASGFTLSSYAMHWVR | STSA-C27- |
| QAPGKGLEYVSGISHSGNSTYYANSVKGRFTISRDNPK | 59-59-VH |
| NTLFLQMSSLRAEDTAVYYCVRVKRRYRGGMDVWGQGT | SEQ ID NO. |
| TVTVSS | 241 |
| EVQLLESGGGLVQPGGSLRLSCAASGFTLSSYAMHWVR | STSA-C27- |
| QAPGKGLEYVSGISPSSNSTYYANSVKGRFTISRDNPK | 58-58-VH |
| NTLFLQMSSLRAEDTAVYYCVRVKVRYRGGMDVWGQGT | SEQ ID NO. |
| TVTVSS | 242 |
| EVQLLESGGGLVQPGGSLRLSCAASGFTLSSYAMHWVR | STSA-C27- |
| QAPGKGLEYVSGISSSGSSTYYANSVKGRFTISRDNPK | 52-52-VH |
| NTLFLQMSSLRAEDTAVYYCVRVKPAYRGGMDVWGQGT | SEQ ID NO. |
| TVTVSS | 243 |
| EVQLLESGGGLVQPGGSLRLSCAASGFTLSSYAMHWVR | STSA-C27- |
| QAPGKGLEYVSGISYSSASTYYANSVKGRFTISRDNPK | Y2-Y2-VH |
| NTLFLQMSSLRAEDTAVYYCVRVKVRYRGGMDVWGQGT | SEQ ID NO. |
| TVTVSS | 244 |
| ETTLTQSPDTLPLSPGDRASLSCRASQSVSSAYLAWYQ | STSA-C27- |
| QKPGQAPRLLIYGTSRRATGVPGRFSGSGSGTDFTLTI | VL SEQ ID |
| SRLEPEDFAVYYCQLYGSSSVTFGQGTKLEIK | NO. 245 |
| EIVLTQSPGTLSLSPGERATLSCRASQGISSAYLAWYQ | STSA-C27- |
| QKPGQAPRLLIYGTSRRATGIPDRFSGSGSGTDFTLTI | 6-33-VL |
| SRLEPEDFAVYYCQLYGATSVTFGQGTKLEIK | SEQ ID NO. |
| | 246 |
| EIVLTQSPGTLSLSPGERATLSCRASQGISSAYLAWYQ | STSA-C27- |
| QKPGQAPRLLIYGTSRRATGIPDRFSGSGSGTDFTLTI | 7-33-VL |
| SRLEPEDFAVYYCQLYGATSVTFGQGTKLEIK | SEQ ID NO. |
| | 247 |
| EIVLTQSPGTLSLSPGERATLSCRASQSVSSAYLAWYQ | STSA-C27- |
| QKPGQAPRLLIYGTSRRATGIPDRFSGSGSGTDFTLTI | 24-56-VL |
| SRLEPEDFAVYYCQLYGASSVTFGQGTKLEIK | SEQ ID NO. |
| | 248 |
| EIVLTQSPGTLSLSPGERATLSCRASQSVSSAYLAWYQ | STSA-C27- |
| QKPGQAPRLLIYGTSRRATGIPDRFSGSGSGTDFTLTI | 47-56-VL |
| SRLEPEDFAVYYCQLYGASSVTFGQGTKLEIK | SEQ ID NO. |
| | 249 |
| EIVLTQSPGTLSLSPGERATLSCRASQGISSAYLAWYQ | STSA-C27- |
| QKPGQAPRLLIYGTSRRATGIPDRFSGSGSGTDFTLTI | 33-33-VL |
| SRLEPEDFAVYYCQLYGATSVTFGQGTKLEIK | SEQ ID NO. |
| | 250 |
| EIVLTQSPGTLSLSPGERATLSCRASQSVSSAYLAWYQ | STSA-C27- |
| QKPGQAPRLLIYGTSRRATGIPDRFSGSGSGTDFTLTI | 56-56-VL |
| SRLEPEDFAVYYCQLYGASSVTFGQGTKLEIK | SEQ ID NO. |
| | 251 |
| EIVLTQSPGTLSLSPGERATLSCRASQSISTAYLAWYQ | STSA-C27- |
| QKPGQAPRLLIYGTSRRATGIPDRFSGSGSGTDFTLTI | 78-78-VL |
| SRLEPEDFAVYYCQLYGASSVTFGQGTKLEIK | SEQ ID NO. |
| | 252 |
| EIVLTQSPGTLSLSPGERATLSCRASQDISSAYLAWYQ | STSA-C27- |
| QKPGQAPRLLIYGTSRRATGIPDRFSGSGSGTDFTLTI | 82-58-VL |
| SRLEPEDFAVYYCQLYGATSVTFGQGTKLEIK | SEQ ID NO. |
| | 253 |
| EIVLTQSPGTLSLSPGERATLSCRASQDVSSAYLAWYQ | STSA-C27- |
| QKPGQAPRLLIYGTSRRATGIPDRFSGSGSGTDFTLTI | 54-54-VL |
| SRLEPEDFAVYYCQLYGATSVTFGQGTKLEIK | SEQ ID NO. |
| | 254 |
| EIVLTQSPGTLSLSPGERATLSCRASQNISTAYLAWYQ | STSA-C27- |
| QKPGQAPRLLIYGTSRRATGIPDRFSGSGSGTDFTLTI | 36-36-VL |
| SRLEPEDFAVYYCQLYGATSVTFGQGTKLEIK | SEQ ID NO. |
| | 255 |
| EIVLTQSPGTLSLSPGERATLSCRASQDASNAYLAWYQ | STSA-C27- |
| QKPGQAPRLLIYGTSRRATGIPDRFSGSGSGTDFTLTI | 53-53-VL |
| SRLEPEDFAVYYCQLYGSSSVTFGQGTKLEIK | SEQ ID NO. |
| | 256 |
| EIVLTQSPGTLSLSPGERATLSCRASQGVSSAYLAWYQ | STSA-C27- |
| QKPGQAPRLLIYGTSRRATGIPDRFSGSGSGTDFTLTI | 67-67-VL |
| SRLEPEDFAVYYCQLYGRSSVTFGQGTKLEIK | SEQ ID NO. |
| | 257 |
| EIVLTQSPGTLSLSPGERATLSCRASQNISTAYLAWYQ | STSA-C27- |
| QKPGQAPRLLIYGTSRRATGIPDRFSGSGSGTDFTLTI | 55-55-VL |
| SRLEPEDFAVYYCQLYGTSSVTFGQGTKLEIK | SEQ ID NO. |
| | 258 |
| EIVLTQSPGTLSLSPGERATLSCRASQSVSTAYLAWYQ | STSA-C27- |
| QKPGQAPRLLIYGTSRRATGIPDRFSGSGSGTDFTLTI | 59-59-VL |
| SRLEPEDFAVYYCQLYGATSVTFGQGTKLEIK | SEQ ID NO. |
| | 259 |
| EIVLTQSPGTLSLSPGERATLSCRASQDISSAYLAWYQ | STSA-C27- |
| QKPGQAPRLLIYGTSRRATGIPDRFSGSGSGTDFTLTI | 58-58-VL |
| SRLEPEDFAVYYCQLYGATSVTFGQGTKLEIK | SEQ ID NO. |
| | 260 |
| EIVLTQSPGTLSLSPGERATLSCRASQGVSTAYLAWYQ | STSA-C27- |
| QKPGQAPRLLIYGTSRRATGIPDRFSGSGSGTDFTLTI | 52-52-VL |
| SRLEPEDFAVYYCQLYGATSVTFGQGTKLEIK | SEQ ID NO. |
| | 261 |
| EIVLPQSPGTLSLSPGERATLSCRASQGVSSAYLAWYQ | STSA-C27- |
| QKPGQAPRLLIYGTSRRATGIPDRFSGSGSGTDFTLTI | Y2-Y2-VL |
| SRLEPEDFAVYYCQLYGSTSVTFGQGTKLEIK | SEQ ID NO. |
| | 262 |

In certain embodiments, an antibody or antigen-binding fragment thereof of the disclosure comprises heavy chain variable region (HCVR) and light chain variable region (LCVR) sequence pairs (HCVR/LCVR) selected from the group consisting of: Y0188-1/Y0188-1; Y0188-2/Y0188-2; Y0188-3/Y0188-3; Y0188-4/Y0188-4; Y0188-6/Y0188-6; Y0188-8/Y0188-8; Y0188-9/Y0188-9; Y0188-10/Y0188-10; Y0188-14/Y0188-14; HV3-15-14/Y01-14; HV3-15-14/164-14; HV3-15-14/KV4-14; HV3-15-14/KV1-27-14; HV3-15-14/KV1-9-14; HV3-15-14/KV1-NL1-14; HV3-15-14/KV1D-43-14; HV3-48-14/Y01-14; HV3-48-14/164-14; HV3-48-14/KV4-14; HV3-48-14/KV1-27-14; HV3-48-14/KV1-9-14; HV3-48-14/KV1-NL1-14; HV3-48-14/KV1D-43-14; HV3-73*2-14/Y01-14; HV3-73*2-14/164-14; HV3-73*2-14/KV4-14; HV3-73*2-14/KV1-27-14; HV3-73*2-14/KV1-9-14; HV3-73*2-14/KV1-NL1-14; HV3-73*2-14/KV1D-43-14; HV3-72-14/Y01-14; HV3-72-14/164-14; HV3-72-14/KV4-14; HV3-72-14/KV1-27-14; HV3-72-14/KV1-9-14; HV3-72-14/KV1-NL1-14; HV3-72-14/KV1D-43-14; Y01-14/Y01-14; Y01-14/164-14; Y01-14/KV4-14; Y01-14/KV1-27-14; Y01-14/KV1-9-14; Y01-14/KV1-NL1-14; Y01-14/KV1D-43-14; 162-14/Y01-14; 162-14/164-14; 162-14/KV4-14; 162-14/KV1-27-14; 162-14/KV1-9-14; 162-14/KV1-NL1-14; 162-14/KV1D-43-1L; VH73-14/Y01-14; VH73-14/164-14; VH73-14/KV4-14; VH73-14/KV1-27-14; VH73-14/KV1-9-14; V_{H}73-14/KV1-NL1-14; and VH73-14/KV1D-43-14.

The antibodies recited below in Table 6 are described in more detail in WO2021/213329, incorporated herein by reference in its entirety for all purposes.

TABLE 6

| | |
|---|---|
| Y0188-1 | EVQLVESGGGLVQPKGSLKLSCAASGFTFNTYGMHWVR |
| VH SEQ ID | QAPGKGLEWVAHIRSKSSNYATYYADSVKDRFTISRDD |
| NO. 263 | SQSMLYLQMNNLKTEDTAMYYCVRWFRAMDYWGQGTSV |
| | TVSS |
| Y0188-2 | EVQLIESGGGLVQPKGSLKLSCAASGFTFNMYAMDWVR |
| VH SEQ ID | QAPGKGLEWVARIRSKGSNFETNYADSVKDRFTISRDD |
| NO. 264 | SQSMVYLQMINLKTEDTAMYYCVRHRGGAWFAYWGQGT |
| | LVSVSA |
| Y0188-3 | QVQLVETGGGLVRPGNSLKLSCVTSGFTFSNYRMHWLR |
| VH SEQ ID | QPPGKRLEWIAVITVKSNNYGANYAESVKGRFAISRDD |
| NO. 265 | SKSSVYLEMNRLREEDTATYFCSRERAYGNPFDYWGQG |
| | TTLTVSS |
| Y0188-4 | EVQLVESGGGLVQPKGSLKLSCAASGFTFNMYAMNWVR |
| VH SEQ ID | QAPGQGLEWVARIRSKSNNYATYYADSVKDRFIISRDD |
| NO. 266 | SESMVYLQMSNLRAADTAMYYCVRHLRAMDYWGQGTSV |
| | TVSS |
| Y0188-6 | EVQLVESGGGLVQPKGSLKLSCAASGFSFNMYAMNWVR |
| VH SEQ ID | QAPGKGLEWVARIRTKSNHYSTYYADSVKDRFTISRDD |
| NO. 267 | SASMFYLQMNNLKTEDTAMYFCVRHLRAMDYWGQGTSV |
| | TVSS |
| Y0188-8 | EVQLIESGGGLVQPKGSLKLSCAASGFTFNMYAMDWVR |
| VH SEQ ID | QAPGKGLEWVARIRSKGSNFETNYADSVKDRFTISRDD |
| NO. 268 | SQSMVYLQMNNLKTEDTAMYYCVRHRGGAWFAYWGQGT |
| | LVTVSA |
| Y0188-9 | EVQLVESGGGLVRPKGSLKLSCAASGFSFNTYAMNWVR |
| VH SEQ ID | QAPGKGLEWIVWIRSKSHNYATYYADSVKDRFTISRDD |
| NO. 269 | SESMLYLQMNNLKTEDTAMYYCVRHLRAMDYWGQGTSV |
| | TVSS |
| Y0188-10 | EVRLVESGGGLVQPKGSLKLSCEASGFSFNMYAMNWVR |
| VH SEQ ID | QAPGKGLEWITHIRSKSNNYATYYADSVKDRFIISRDD |
| NO. 270 | SESMVYLQMNNLKTEDTAMYYCVRLLRALDYWGQGTSV |
| | TVSS |
| Y0188-14 | EVQLVESGGGLVQPKGSLKLSCAASGFTFNMYGMHWVR |
| VH SEQ ID | QAPGKGLEWVAHIRSKSSNYATYYADSVKDRLTISRDD |
| NO. 271 | SQSMLYLQMNNLKTEDTAMYYCVRWFRAMDYWGQGTSV |
| | TVSS |
| HV3-15-14 | EVQLVESGGGLVKPGGSLRLSCAASGFTFSMYGMHWVR |
| VH SEQ ID | QAPGKGLEWVGHIRSKSSNYATYYADSVKDRFTISRDD |
| NO. 272 | SKNTLYLQMNSLKTEDTAVYYCTTWFRAMDYWGQGTLV |
| | TVSS |
| HV3-48-14 | EVQLVESGGGLVQPGGSLRLSCAASGFTFSMYGMHWVR |
| VH SEQ ID | QAPGKGLEWVSHIRSKSSNYATYYADSVKDRFTISRDN |
| NO. 273 | AKNSLYLQMNSLRAEDTAVYYCARWFRAMDYWGQGTLV |
| | TVSS |
| HV3-73*2- | EVQLVESGGGLVQPGGSLKLSCAASGFTFSMYGMHWVR |
| 14 VH | QASGKGLEWVGHIRSKSSNYATYYADSVKDRFTISRDD |
| SEQ ID | SKNTAYLQMNSLKTEDTAVYYCTRWFRAMDYWGQGTLV |
| NO. 274 | TVSS |
| HV3-72-14 | EVQLVESGGGLVQPGGSLRLSCAASGFTFSMYGMHWVR |
| VH SEQ ID | QAPGKGLEWVGHIRSKSSNYATYYADSVKDRFTISRDD |
| NO. 275 | SKNSLYLQMNSLKTEDTAVYYCARWFRAMDYWGQGTLV |
| | TVSS |
| Y01-14 | EVQLVESGGGLVQPGGSLRLSCAASGFTFSMYGMHWVR |
| VH SEQ ID | QAPGKGLEWVSHIRSKSSNYATYYADSVKDRFTISRDN |
| NO. 276 | AKNSLYLQMNSLRAEDTAVYYCARWFRAMDYWGQGTLV |
| | TVSS |
| 162-14 | EVQLVESGGGLEQPGGSLRLSCAGSGFTFRMYGMHWVR |
| VH SEQ ID | QAPGKGLEWVSHIRSKSSNYATYYADSVKDRFTISRDN |
| NO. 277 | SKNTLYLQMNSLRAEDTAVYYCAKWFRAMDYWGQGTTV |
| | TVSS |
| VH 73-14 | EVQLVESGGGLVQPGGSLKLSCAASGFTFSMYGMHWVR |
| VH SEQ ID | QASGKGLEWVGHIRSKSSNYATYYADSVKDRFTISRDD |
| NO. 278 | SKNTAYLQMNSLKTEDTAVYYCTRWFRAMDYWGQGTTV |
| | TVSS |
| Y0188-1 | DIVMTQSHKFMSTSVGDRVSITCKASQDVSTAVAWYQE |
| VL SEQ ID | KPGQSPKLLIYWASTRHTGVPDRFTGSGSGTDYTLTIS |
| NO. 279 | SVQAEDLALYYCQQHYSTPLTFGAGTKLELK |
| Y0188-2 | DIVVTQSPASLAVSLGQRATISCRASKSVSTSGYSYMH |
| VL SEQ ID | WYQQKPGQPPKLLIYLASNLESGVPARFSGSGSGTDFT |
| NO. 280 | LNIHPVEEEDVAIYYCQHSRELPLTFGAGTKLELK |
| Y0188-3 | DIQMTQSPSSLSASLGERVSLTCRASQEISGYLSWLQQ |
| VL SEQ ID | KPDGTIKRLIYAASTLDSGVPKRFSGSRSGSDYSLTIS |
| NO. 281 | SLESEDFADYYCLQYGSYPYTFGGGTKLEIK |
| Y0188-4 | DIVLTQSPASLTVSLGQRATISCRASKSVSTSGYSYMH |
| VL SEQ ID | WYQQKPGQPPKLLIYLASNLESGVPARFSGSGSGTDFT |
| NO. 282 | LNIHPVEEEDAATYYCQHSRELPITFGSGTKLEIK |
| Y0188-6 | DIVLTQSPASLVVSLGQRATISCRASQSVSTSGYSYMH |
| VL SEQ ID | WYQQKPGQPPKLLIYLASNVQSGVPARFSGSGSGTDFT |
| NO. 283 | LNIHPVEEEDVATYYCHHNRDLPFTFGSGTKLEIK |
| Y0188-8 | DIVVTQSPASLAVSLGQRATISCRASKSVSTSGYSYMH |
| VL SEQ ID | WYQQKPGQPPKLLIYLASNLESGVPARFSGSGSGTDFT |
| NO. 284 | LNIHPVEEEDVAIYYCQHSRELPLTFGAGTKLELK |
| Y0188-9 | DIVLTQSPASLAVSLGQRATISCRASKSVSASGYSYMH |
| VL SEQ ID | WYQQKPGQPPKLLIYLASNLQSGVPARFSGSGSGTDFT |
| NO. 286 | LNIHPVEEEDAATYYCQHSRELPPTFGGGTKLEIK |
| Y0188-10 | DIVLTQSPASLAVFLGQRATISCRASKSVSTSGYSYMH |
| VL SEQ ID | WYQQKAGQPPKLLIYLASNLESGVPARFSGSGSGTDFT |
| NO. 287 | LNIHPVEEEDAATYYCHHSRELPITFGSGTKLEMK |
| Y0188-14 | DIVMTQSHKFMSTSVGDRVSITCKASQDVSTAVAWYQE |
| VL SEQ ID | KPGQSPKLLIYWASTRHTGVPDRFTGSGSGTDYTLTIS |
| NO. 288 | SVQAEDLALYYCQQHYSTPLTFGAGTKLELK |
| Y01-14 | EIVLTQSPGTLSLSPGERATLSCKASQDVSTAVAWYQQ |
| VL SEQ ID | KPGQAPRLLIYWASTRHTGIPDRFSGSGSGTDFTLTIS |
| NO. 289 | RLEPEDFAVYYCQQHYSTPLTFGQGTKVEIK |
| 164-14 | DIVMTQSPLSLPVTPGEPASISCKASQDVSTAVAWYLQ |
| VL SEQ ID | KSGQSPQLLIYWASTRHTGVPDRFSGSGSGTDFTLKIS |
| NO. 290 | RVEAEDVGFYYCQQHYSTPLTFGQGTKLEIK |
| KV4-14 | DIVMTQSPDSLAVSLGERATINCKASQDVSTAVAWYQQ |
| VL SEQ ID | KPGQPPKLLIYWASTRHTGVPDRFSGSGSGTDFTLTIS |
| NO. 291 | SLQAEDVAVYYCQQHYSTPLTFGGGTKVEIK |
| KV1-27-14 | DIQMTQSPSSLSASVGDRVTITCKASQDVSTAVAWYQQ |
| VL SEQ ID | KPGKVPKLLIYWASTRHTGVPSRFSGSGSGTDFTLTIS |
| NO. 292 | SLQPEDVATYYCQQHYSTPLTFGGGTKVEIK |
| KV1-9-14 | DIQLTQSPSFLSASVGDRVTITCKASQDVSTAVAWYQQ |
| VL SEQ ID | KPGKAPKLLIYWASTRHTGVPSRFSGSGSGTEFTLTIS |
| NO. 293 | SLQPEDFATYYCQQHYSTPLTFGGGTKVEIK |
| KV1-NL1- | DIQMTQSPSSLSASVGDRVTITCKASQDVSTAVAWYQQ |
| 14 VL | KPGKAPKLLLYWASTRHTGVPSRFSGSGSGTDYTLTIS |
| SEQ ID | SLQPEDFATYYCQQHYSTPLTFGGGTKVEIK |
| NO. 294 | |
| KV1D-43- | AIRMTQSPFSLSASVGDRVTITCKASQDVSTAVAWYQQ |
| 14 VL | KPAKAPKLFIYWASTRHTGVPSRFSGSGSGTDYTLTIS |
| SEQ ID | SLQPEDFATYYCQQHYSTPLTFGGGTKVEIK |
| NO. 295 | |

### Pharmaceutical Compositions

Methods that comprise administering an IL-4R antagonist to a patient, wherein the IL-4R antagonist is contained within a pharmaceutical composition are provided. The pharmaceutical compositions described herein are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol. 52:238-311.

The dose of antibody administered to a patient may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-IL-4R antibodies may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical compositions described herein, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, intra-tracheal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition described herein can be delivered subcutaneously or intravenously with a standard needle and syringe. In addition, with respect to subcutaneous delivery, a pen delivery device (e.g., an autoinjector pen) readily has applications in delivering a pharmaceutical composition described herein. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition described herein include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.), and the HUMIRA™ Pen (Abbott Labs, Abbott Park Ill.), to name only a few. Examples of large-volume delivery devices (e.g., large-volume injectors) include, but are not limited to, bolus injectors such as, e.g., BD Libertas West SmartDose, Enable Injections, SteadyMed PatchPump, Sensile SenseTrial, YPsomed YpsoDose, Bespak Lapas, and the like.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

An example sleeve-triggered auto-injector with manual needle insertion is described in International Publication WO2015/004052. Example audible end-of-dose feedback mechanisms are described in International Publications WO2016/193346 and WO2016/193348. An example needle-safety mechanism after using an auto-injector is described in International Publication WO2016/193352. An example needle sheath remover mechanism for a syringe auto-injector is described in International Publication WO2016/193353. An example support mechanism for supporting an axial position of a syringe is described in International Publication WO2016/193355.

For direct administration to the sinuses, the pharmaceutical compositions described herein may be administered using, e.g., a microcatheter (e.g., an endoscope and microcatheter), an aerosolizer, a powder dispenser, a nebulizer or an inhaler. The methods include administration of an IL-4R antagonist to a subject in need thereof, in an aerosolized formulation. For example, aerosolized antibodies to IL-4R may be administered to treat PN in a patient. Aerosolized antibodies can be prepared as described in, for example, U.S. Pat. No. 8,178,098, incorporated herein by reference in its entirety.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mol) adduct of hydrogenated castor oil)), etc. As the oily medium, there are employed, e.g., sesame oil, soybean oil, etc., which may be used in combination with a solubilizing agent such as benzyl benzoate, benzyl alcohol, etc. The injection thus prepared is typically filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

Exemplary pharmaceutical compositions comprising an anti-IL-4R antibody that can be used as described herein are disclosed, e.g., in U.S. Pat. No. 8,945,559.

### Dosage

The amount of IL-4R antagonist (e.g., anti-IL-4R antibody) administered to a subject according to the methods described herein is, generally, a therapeutically effective amount. As used herein, the phrase “therapeutically effective amount” means an amount of IL-4R antagonist that results in improvement in one or more PN-associated PRO measures or PN-associated ClinRO measures (as defined elsewhere herein). A “therapeutically effective amount” also includes an amount of IL-4R antagonist that inhibits, prevents, lessens, or delays the progression of PN in a subject.

In the case of an anti-IL-4R antibody, a therapeutically effective amount can be from about 0.05 mg to about 700 mg, e.g., about 0.05 mg, about 0.1 mg, about 1.0 mg, about 1.5 mg, about 2.0 mg, about 3.0 mg, about 5.0 mg, about 7.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, or about 700 mg of the anti-IL-4R antibody. In certain embodiments, 300 mg of an anti-IL-4R antibody is administered.

The amount of IL-4R antagonist contained within the individual doses may be expressed in terms of milligrams of antibody per kilogram of subject body weight (i.e., mg/kg). For example, the IL-4R antagonist may be administered to a patient at a dose of about 0.0001 to about 10 mg/kg of subject body weight. For example, the IL-4R antagonist can be administered at a dose of 1 mg/kg, 2 mg/kg, 3 mg/kg, 4 mg/kg, 5 mg/kg or 6 mg/kg.

In certain embodiments, the initial dose is about the same as the loading dose. In certain embodiments, the initial dose is about 1.1×, about 1.2×, about 1.3×, about 1.4×, about 1.5×, about 1.6×, about 1.7×, about 1.8×, about 1.9×, about 2.0×, about 2.5×, about 3.0×, or more of the loading dose.

In certain embodiments, two or more (e.g., 2, 3, 4, or 5 or more) doses are administered at the beginning of the treatment regimen as “initial doses” or “loading doses” followed by subsequent doses that are administered on a less frequent basis (e.g., “secondary doses” or “maintenance doses”). In one embodiment, the maintenance dose may be lower than the loading or initial dose. For example, one or more loading doses of 600 mg of IL-4R antagonist may be administered followed by maintenance doses of about 75 mg to about 300 mg. In certain embodiments, the methods comprise an initial dose or loading dose of about 400 mg or about 600 mg of an IL-4R antagonist. In certain embodiments, the methods comprise one or more secondary doses or maintenance doses of about 200 mg or about 300 mg of the IL-4R antagonist.

In one embodiment, the maintenance dose is the same dose as the loading or initial dose. For example, both the loading dose and the maintenance doses of the IL-4R antagonist may be administered in doses of about 75 mg to about 300 mg. In certain embodiments, the methods comprise an initial dose and maintenance doses of about 300 mg of an IL-4R antagonist.

In certain exemplary embodiments, a subject is a pediatric subject having a body weight of more than 30 kg, and the IL-4R antagonist is administered at a dose of about 150 mg, about 200 mg, about 250 mg, about 300 mg, about 350 mg, about 400 mg, about 450 mg, about 500 mg, about 550 mg, or about 600 mg. In some embodiments, a subject is a pediatric subject having a body weight of more than 30 kg, and the IL-4R antagonist is administered at an initial dose or loading dose of about 400 mg and one or more secondary doses or maintenance doses of about 200 mg, and the secondary doses are administered every other week (q2w). In some embodiments, a subject is a pediatric subject having a body weight of more than 30 kg, and the IL-4R antagonist is administered at an initial dose and maintenance doses of about 200 mg, and the maintenance doses are administered every other week (q2w).

In certain exemplary embodiments, a subject is a pediatric subject having a body weight of 30 kg or less and a body weight of at least 15 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In some embodiments, a subject is a pediatric subject having a body weight of 30 kg or less and a body weight of at least 15 kg, and the IL-4R antagonist is administered at an initial dose of about 600 mg and one or more secondary doses or maintenance doses of about 300 mg, and the secondary doses are administered every four weeks (q4w). In some embodiments, a subject is a pediatric subject having a body weight of 30 kg or less and a body weight of at least 15 kg, and the IL-4R antagonist is administered at an initial dose and maintenance doses of about 300 mg, and the maintenance doses are administered every four weeks (q4w).

In certain exemplary embodiments, a subject is an adolescent subject having a body weight of less than 60 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In some embodiments, a subject is an adolescent subject having a body weight of less than 60 kg, and the IL-4R antagonist is administered at an initial dose of about 400 mg and one or more secondary doses or maintenance doses of about 200 mg, and the secondary doses are administered every other week (q2w). In other embodiments, a subject is an adolescent subject having a body weight of less than 60 kg, and the IL-4R antagonist is administered at an initial dose and maintenance doses of about 200 mg, and the maintenance doses are administered every other week (q2w). In certain embodiments, a subject is an adolescent subject having a body weight that is greater than or equal to 30 kg and less than 60 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In some embodiments, a subject is an adolescent subject having a body weight that is greater than or equal to 30 kg and less than 60 kg, and the IL-4R antagonist is administered at an initial dose of about 400 mg and one or more secondary doses or maintenance doses of about 200 mg, and the secondary doses are administered every other week (q2w). In other embodiments, a subject is an adolescent subject having a body weight that is greater than or equal to 30 kg and less than 60 kg, and the IL-4R antagonist is administered at an initial dose and maintenance doses of about 200 mg, and the maintenance doses are administered every other week (q2w).

In certain exemplary embodiments, a subject is an adolescent subject having a body weight of at least 60 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In exemplary embodiments, a subject is an adolescent subject having a body weight of at least 60 kg, and the IL-4R antagonist is administered at an initial dose of about 600 mg and one or more secondary doses or maintenance doses of about 300 mg, and the secondary doses are administered every other week (q2w). In other embodiments, a subject is an adolescent subject having a body weight of at least 60 kg, and the IL-4R antagonist is administered at an initial dose and maintenance doses of about 300 mg, and the maintenance doses are administered every other week (q2w).

In certain exemplary embodiments, a subject is an adult, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In exemplary embodiments, a subject is an adult, and the IL-4R antagonist is administered at an initial dose of about 600 mg and one or more secondary doses or maintenance doses of about 300 mg, and the secondary doses are administered every other week (q2w). In other exemplary embodiments, a subject is an adult, and the IL-4R antagonist is administered at an initial dose of about 300 mg and maintenance doses of about 300 mg, and the maintenance doses are administered every other week (q2w).

In certain exemplary embodiments, an IL-4R antagonist is administered at a concentration of 150 mg/mL using a prefilled device. In some embodiments, a 150 mg/mL IL-4R antagonist solution in a pre-filled device is used to deliver 300 mg IL-4R antagonist in a 2 mL injection. In certain exemplary embodiments, an IL-4R antagonist is administered at a concentration of 175 mg/mL using a prefilled device. In some embodiments, a 175 mg/mL IL-4R antagonist solution in a pre-filled device is used to deliver 200 mg IL-4R antagonist in a 1.14 mL injection.

### Combination Therapies

Certain embodiments of the methods described herein comprise administering to the subject one or more additional therapeutic agents in combination with the IL-4R antagonist. As used herein, the expression “in combination with” means that the additional therapeutic agents are administered before, after, or concurrent with the pharmaceutical composition comprising the IL-4R antagonist. In some embodiments, the term “in combination with” includes sequential or concomitant administration of an IL-4R antagonist and a second therapeutic agent. Methods to treat PN or an associated condition or complication comprising administration of an IL-4R antagonist in combination with a second therapeutic agent for additive or synergistic activity, are provided.

For example, when administered “before” the pharmaceutical composition comprising the IL-4R antagonist, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes, or about 10 minutes prior to the administration of the pharmaceutical composition comprising the IL-4R antagonist. When administered “after” the pharmaceutical composition comprising the IL-4R antagonist, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, or about 72 hours after the administration of the pharmaceutical composition comprising the IL-4R antagonist. Administration “concurrent” with the pharmaceutical composition comprising the IL-4R antagonist means that the additional therapeutic agent is administered to the subject in a separate dosage form within less than 5 minutes (before, after, or at the same time) of administration of the pharmaceutical composition comprising the IL-4R antagonist, or administered to the subject as a single combined dosage formulation comprising both the additional therapeutic agent and the IL-4R antagonist.

In exemplary embodiments, an additional therapeutic agent administered in combination with the IL-4R antagonist is a background therapy. In exemplary embodiments, the background therapy is one or more topical corticosteroids (TCS). In exemplary embodiments, the background therapy is one or more oral corticosteroids (OCS). In other exemplary embodiments, the background therapy is one or more topical calcineurin inhibitors (TCI). In some exemplary embodiments, the background therapy is one or more low to medium potency topical corticosteroids (TCS). In other exemplary embodiments, the background therapy is one or more low to medium potency topical calcineurin inhibitors (TCI). In certain embodiments, the method leads to reduced need of the background therapy. For example, in certain embodiments, the method leads to reduced dose and/or reduced frequency of the background therapy.

The additional therapeutic agent may be, e.g., another IL-4R antagonist (e.g., one or more suitable IL-4R antagonists listed in Tables 1-4), a TCS, a TCI, an IL-1 antagonist (including, e.g., an IL-1 antagonist as set forth in U.S. Pat. No. 6,927,044), an IL-5 antagonist, an IL-5R antagonist, an IL-6 antagonist, an IL-6R antagonist (including, e.g., an anti-IL-6R antibody as set forth in U.S. Pat. No. 7,582,298), or an IL-17 antagonist.

In an exemplary embodiment, the additional therapeutic agent is a medium to superpotent TCS.

In another exemplary embodiment, the additional therapeutic agent is a low to medium potency TCS.

Suitable super-high potency (i.e., group 1) TCSs include, but are not limited to, betamethasone dipropionate (augmented), clobetasol propionate, diflucortolone valerate, fluocinonide, flurandrenolide, halobetasol propionate and the like.

Suitable high potency (i.e., group 2) TCSs include, but are not limited to, amcinonide, betamethasone dipropionate, clobetasol propionate, desoximetasone, diflorasone diacetate, fluocinonide, halcinonide, halobetasol propionate and the like.

Suitable high potency (i.e., group 3) TCSs include, but are not limited to, amcinonide, betamethasone dipropionate, betamethasone valerate, desoximetasone, diflorasone diacetate, diflucortolone valerate, fluocinonide, fluticasone propionate, mometasone furoate, mometasone furoate and the like.

Suitable medium potency (i.e., group 4) TCSs include, but are not limited to, betamethasone dipropionate, clocortolone pivalate, fluocinolone acetonide, flurandrenolide, fluticasone propionate, hydrocortisone valerate, mometasone furoate, triamcinolone acetonide and the like. Particularly suitable medium potency TCSs include triamcinolone acetonide 0.1% cream and fluocinolone acetonide 0.025% ointment.

Suitable lower mid-potency (i.e., group 5) TCSs include, but are not limited to, betamethasone dipropionate, betamethasone valerate, desonide, fluocinolone acetonide, flurandrenolide, fluticasone propionate, hydrocortisone butyrate, hydrocortisone probutate, hydrocortisone valerate, prednicarbate, triamcinolone acetonide and the like.

Suitable low potency (i.e., group 6) TCSs include, but are not limited to, alclometasone dipropionate, betamethasone valerate, desonide, fluocinolone acetonide, triamcinolone acetonide, and the like.

Suitable least potent (i.e., group 7) TCSs include, but are not limited to, hydrocortisone (base, ≥2%), hydrocortisone (base, <2%), hydrocortisone acetate, and the like. A particularly suitable least potent TCSs is hydrocortisone 1% cream.

In a further exemplary embodiment, the additional therapeutic agent is a medium to superpotent TCI.

In a further exemplary embodiment, the additional therapeutic agent is a low to medium potency TCI.

Suitable TCIs include, but are not limited to, clobetasol propionate, betamethasone dipropionate, ASTAGRAF XL™ (i.e., tacrolimus extended-release capsules), CEQUA™ (i.e., cyclosporine 0.09% eye drops), cyclosporine, cyclosporine ophthalmic, ELIDEL™ (i.e., pimecrolimus), ENVARSUS XR™ (i.e., tacrolimus extended-release tablets), GENGRAF™ (i.e., cyclosporine), HECORIA™ (i.e., tacrolimus), LUPKYNIS™ (i.e., voclosporin), NEORAL™ (i.e., cyclosporine capsules and oral solution), pimecrolimus, PROGRAF™ (i.e., tacrolimus capsules), PROTOPIC™ (i.e., tacrolimus ointment), RESTASIS™ (i e, cyclosporine 0.05% eye drops), SANDIMMUNE™ (i.e., cyclosporine capsules and oral solution), tacrolimus, tacrolimus ointment, VERKAZIA™ (i.e., cyclosporine ophthalmic emulsion), voclosporin, and the like.

A particularly suitable super high potency TCS is clobetasol propionate 0.05% cream. A particularly suitable high potency TCS is betamethasone dipropionate 0.05% optimized ointment.

In a further exemplary embodiment, the additional therapeutic agent is an oral corticosteroid (i.e., a systemic corticosteroid).

Suitable oral corticosteroids include, but are not limited to, prednisone, prednisolone, methylprednisolone, hydrocortisone, dexamethasone, cortisone acetate and the like.

### Administration Regimens

According to certain embodiments, multiple doses of an IL-4R antagonist may be administered to a subject over a defined time course. Such methods comprise sequentially administering to a subject multiple doses of an IL-4R antagonist. As used herein, “sequentially administering” means that each dose of IL-4R antagonist is administered to the subject at a different point in time, e.g., on different days separated by a predetermined interval (e.g., hours, days, weeks, or months). Methods that comprise sequentially administering to the patient a single initial dose of an IL-4R antagonist, followed by one or more secondary doses of the IL-4R antagonist, and optionally followed by one or more tertiary doses of the IL-4R antagonist, are provided.

Methods comprising administering to a subject a pharmaceutical composition comprising an IL-4R antagonist at a dosing frequency of about four times a week, twice a week, once a week (q1w), once every two weeks (every two weeks is used interchangeably with every other week, bi-weekly or q2w), once every three weeks (tri-weekly or q3w), once every four weeks (monthly or q4w), once every five weeks (q5w), once every six weeks (q6w), once every seven weeks (q7w), once every eight weeks (q8w), once every nine weeks (q9w), once every ten weeks (g10w), once every eleven weeks (q11w), once every twelve weeks (q12w), or less frequently so long as a therapeutic response is achieved, are provided.

In certain embodiments involving the administration of a pharmaceutical composition comprising an anti-IL-4R antibody, once a week dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every two weeks dosing (every two weeks is used interchangeably with every other week, bi-weekly or q2w) of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every three weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every four weeks dosing (monthly dosing) of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every five weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every six weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every eight weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every twelve weeks dosing of an amount of about 100 mg, about 200 mg, about 300 mg, about 400 mg, about 500 mg or about 600 mg can be employed. In certain exemplary embodiments, the route of administration is subcutaneous.

The term “week” or “weeks” refers to a period of (n×7 days)±3 days, e.g., (n×7 days)±2 days, (n×7 days)±1 day, or (n×7 days), wherein “n” designates the number of weeks, e.g. 1, 2, 3, 4, 5, 6, 8, 12 or more.

The terms “initial dose,” “secondary doses,” and “tertiary doses,” refer to the temporal sequence of administration of the IL-4R antagonist. Thus, the “initial dose” is the dose that is administered at the beginning of the treatment regimen (also referred to as the “baseline dose” or “loading dose”); the “secondary doses” are the doses that are administered after the initial dose; and the “tertiary doses” are the doses that are administered after the secondary doses. The initial, secondary, and tertiary doses may all contain the same amount of IL-4R antagonist, or may differ from one another in terms of frequency of administration. In certain embodiments, however, the amount of IL-4R antagonist contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”). In one embodiment, the maintenance dose may be lower than the loading dose. For example, one or more initial doses or loading doses of 600 mg or 400 mg of IL-4R antagonist may be administered followed by secondary doses or maintenance doses of about 75 mg to about 400 mg. In one embodiment, the secondary dose/maintenance dose may be equal to the initial dose/loading dose. For example, one or more initial doses/loading doses of 300 mg or 200 mg of IL-4R antagonist may be administered followed by secondary doses/maintenance doses of about 300 mg or about 200 mg, respectively. In one embodiment, a loading dose may be split, e.g., two or more doses administered at different time points, e.g., two loading doses wherein a second loading dose is administered two weeks after a first loading dose.

In certain embodiments, the initial dose is about 50 mg to about 600 mg of the IL-4R antagonist. In one embodiment, the initial dose is 600 mg of the IL-4R antagonist. In another embodiment, the initial dose is 400 mg of the IL-4R antagonist. In still other embodiments, the initial dose is 300 mg of the IL-4R antagonist.

In certain embodiments, the secondary dose(s) are about 50 mg to about 600 mg of the IL-4R antagonist. In one embodiment, the maintenance dose is 300 mg of the IL-4R antagonist. In one embodiment, the maintenance dose is 200 mg of the IL-4R antagonist.

In certain embodiments, an initial dose is three times a maintenance dose. In certain embodiments, an initial dose is two times a maintenance dose. In certain embodiments, an initial dose is equal to a maintenance dose. In an exemplary embodiment, the initial dose is 300 mg and the maintenance dose(s) are 300 mg.

In some embodiments, the subject is a child and has a body weight of 30 kg or less and at least 15 kg, the initial dose comprises 600 mg of the antibody or antigen-binding fragment thereof, and the one or more secondary doses comprises 300 mg of the antibody or antigen-binding fragment thereof administered every four weeks (q4w). In other embodiments, the subject is a child and has a body weight of 30 kg or less and at least 15 kg, the initial dose comprises 300 mg of the antibody or antigen-binding fragment thereof, and the one or more secondary doses comprises 300 mg of the antibody or antigen-binding fragment thereof administered every four weeks (q4w).

In some embodiments, the subject is a child and has a body weight of greater than 30 kg, the initial dose comprises 400 mg of the antibody or antigen-binding fragment thereof, and the one or more secondary doses comprises 200 mg of the antibody or antigen-binding fragment thereof administered every other week (every other week is used interchangeably with every two weeks, bi-weekly or q2w). In other embodiments, the subject is a child and has a body weight of greater than 30 kg, the initial dose comprises 200 mg of the antibody or antigen-binding fragment thereof, and the one or more secondary doses comprises 200 mg of the antibody or antigen-binding fragment thereof administered every other week (every other week is used interchangeably with every two weeks, bi-weekly or q2w).

In some embodiments, the subject is an adolescent and has a body weight of less than 60 kg, the initial dose comprises 400 mg of the antibody or antigen-binding fragment thereof, and the one or more secondary doses comprises 200 mg of the antibody or antigen-binding fragment thereof administered every other week (every other week is used interchangeably with every two weeks, bi-weekly or q2w). In other embodiments, the subject is an adolescent and has a body weight of less than 60 kg, the initial dose comprises 200 mg of the antibody or antigen-binding fragment thereof, and the one or more secondary doses comprises 200 mg of the antibody or antigen-binding fragment thereof administered every other week (every other week is used interchangeably with every two weeks, bi-weekly or q2w). In exemplary embodiments, the subject is an adolescent and has a body weight that is greater than or equal to 30 kg and less than 60 kg, the initial dose comprises 400 mg of the antibody or antigen-binding fragment thereof, and the one or more secondary doses comprises 200 mg of the antibody or antigen-binding fragment thereof administered every other week (every other week is used interchangeably with every two weeks, bi-weekly or q2w). In other exemplary embodiments, the subject is an adolescent and has a body weight that is greater than or equal to 30 kg and less than 60 kg, the initial dose comprises 200 mg of the antibody or antigen-binding fragment thereof, and the one or more secondary doses comprises 200 mg of the antibody or antigen-binding fragment thereof administered every other week (every other week is used interchangeably with every two weeks, bi-weekly or q2w).

In some embodiments, the subject is an adolescent and has a body weight of more than 60 kg, the initial dose comprises 600 mg of the antibody or antigen-binding fragment thereof, and the one or more secondary doses comprises 300 mg of the antibody or antigen-binding fragment thereof administered every other week (every other week is used interchangeably with every two weeks, bi-weekly or q2w). In other embodiments, the subject is an adolescent and has a body weight of more than 60 kg, the initial dose comprises 300 mg of the antibody or antigen-binding fragment thereof, and the one or more secondary doses comprises 300 mg of the antibody or antigen-binding fragment thereof administered every other week (every other week is used interchangeably with every two weeks, bi-weekly or q2w).

In some embodiments, the subject is an adult, the initial dose comprises 600 mg of the antibody or antigen-binding fragment thereof, and the one or more secondary doses comprises 300 mg of the antibody or antigen-binding fragment thereof administered every other week (every other week is used interchangeably with every two weeks, bi-weekly or q2w). In other embodiments, the subject is an adult, the initial dose comprises 300 mg of the antibody or antigen-binding fragment thereof, and the one or more secondary doses comprises 300 mg of the antibody or antigen-binding fragment thereof administered every other week (every other week is used interchangeably with every two weeks, bi-weekly or q2w).

In one exemplary embodiment, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose” means, in a sequence of multiple administrations, the dose of IL-4R antagonist that is administered to a patient prior to the administration of the very next dose in the sequence with no intervening doses.

The methods may include administering to a patient any number of secondary and/or tertiary doses of an IL-4R antagonist. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

Methods comprising sequential administration of an IL-4R antagonist and a second therapeutic agent, to a patient to treat PN or an associated condition are provided. In some embodiments, the methods comprise administering one or more doses of an IL-4R antagonist followed by one or more doses (e.g., 2, 3, 4, 5, 6, 7, 8, or more) of a second therapeutic agent. For example, one or more doses of about 75 mg to about 600 mg of the IL-4R antagonist may be administered after which one or more doses (e.g., 2, 3, 4, 5, 6, 7, 8, or more) of a second therapeutic agent (e.g., a TCS or a TCI) may be administered to treat, alleviate, reduce or ameliorate one or more symptoms of PN. In some embodiments, the IL-4R antagonist is administered at one or more doses (e.g., 2, 3, 4, 5, 6, 7, 8, or more) resulting in an improvement in one or more PN-associated parameters followed by the administration of a second therapeutic agent to prevent recurrence of at least one symptom of PN. Alternative embodiments pertain to concomitant administration of an IL-4R antagonist and a second therapeutic agent. For example, one or more doses (e.g., 2, 3, 4, 5, 6, 7, 8, or more) of an IL-4R antagonist are administered and a second therapeutic agent is administered at a separate dosage at a similar or different frequency relative to the IL-4R antagonist. In some embodiments, the second therapeutic agent is administered before, after or concurrently with the IL-4R antagonist.

In certain embodiments, the IL-4R antagonist is administered every other week for 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks or more. In other embodiments, the IL-4R antagonist is administered every four weeks for 12 weeks, 16 weeks, 20 weeks, 24 weeks, 28 weeks, 32 weeks, 36 weeks, 40 weeks, 44 weeks, 48 weeks or more. In specific embodiments, the IL-4R antagonist is administered for at least 24 weeks.

In certain embodiments, a kit comprising a dosage form of an antibody, or an antigen-binding fragment thereof, that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, for the treatment of CIndU is provided. In certain embodiments, the antibody or antigen-binding fragment thereof comprises a heavy chain variable region (HCVR) sequence of SEQ ID NO: 1 and a light chain variable region (LCVR) sequence of SEQ ID NO: 2. In certain embodiments, the antibody is dupilumab.

The kit can comprise a label or package insert, wherein the label or package insert comprises instructions for administering the dosage form for the treatment of PN. The instructions can recite a dosing regimen described further herein for the treatment of PN.

### Treatment Populations

The methods provided herein include administering to a subject in need thereof a therapeutic composition comprising an IL-4R antagonist. The expression “a subject in need thereof” means a human or non-human animal that exhibits one or more symptoms or indicia of PN, or who has been diagnosed with PN.

In a related embodiment, a “subject in need thereof” may be a subject who, prior to receiving an IL-4R antagonist, has been prescribed or is currently taking a TCI or a TCS. In some embodiments, the subject is currently taking a low to medium potency TCI or TCS. For example, methods that comprise administering an IL-4R antagonist to a patient who has been taking a regular course of a low to medium potency TCI or TCS for two or more weeks immediately preceding the administration of the IL-4R antagonist (such prior treatments are referred to herein as “background treatments”) are provided.

In yet other embodiments, the amount of the TCI or TCS is gradually decreased prior to or after the start of IL-4R antagonist administration. In other embodiments, the potency of the TCI or TCS is gradually decreased prior to or after the start of IL-4R antagonist administration.

In another exemplary embodiment, a “subject in need thereof” has a diagnosis of PN refractory to TCSs or TCIs prior to receiving the IL-4R antagonist. In some embodiments, the PN symptoms of the subject persist despite treatment with TCSs or TCIs. In still another exemplary embodiment, a “subject in need thereof” has a diagnosis of PN refractory to medium to superpotent TCSs or TCIs prior to receiving the IL-4R antagonist. In some embodiments, the PN symptoms of the subject persist despite treatment with medium to superpotent TCSs or TCIs. In yet another exemplary embodiment, a “subject in need thereof” has a diagnosis of PN refractory to low to medium potency TCSs or TCIs prior to receiving the IL-4R antagonist. In some embodiments, the PN symptoms of the subject persist despite treatment with low to medium potency TCSs or TCIs.

In another embodiment, a “subject in need thereof” is a subject whose PN is not adequately controlled with topical therapies. In other embodiments, a “subject in need thereof” is a subject who has pruritus or PN refractory to topical therapy. In some embodiments, a “subject in need thereof” is a subject for whom topical therapies are not advisable (i.e. the subject experiences adverse effects associated with topical therapies or is on a medication(s) that cannot be combined with topical therapies.) In still other embodiments, a “subject in need thereof” is a subject who is a candidate for systemic therapy for the treatment of PN or pruritus.

In a further exemplary embodiment, a “subject in need thereof” is a subject for whom TCSs or TCIs are not medically advisable (i.e. the subject has an allergy, a history of an adverse reaction, or other medical history wherein administration of TCSs or TCIs are not advisable.)

In some embodiments, a “subject in need thereof” is selected from the group consisting of: a subject age 18 years old or older, a subject 12 years or older, a subject age 12 to 17 years old (12 to <18 years old), a subject age 6 to 11 years old (6 to <12 years old), and a subject age 2 to 5 years old (2 to <6 years old). In some embodiments, a “subject in need thereof” is selected from the group consisting of: an adult, an adolescent, and a child. In some embodiments, a “subject in need thereof” is selected from the group consisting of: an adult age 18 years of age or older, an adolescent age 12 to 17 years old (12 to <18 years old), a child age 6 to 11 years old (6 to <12 years old), and a child age 2 to 5 years old (2 to <6 years old). The subject can be less than 2 years of age, e.g., 12 to 23 months, or 6 to 11 months.

In some embodiments, a “subject in need thereof” may be a subject who has co-morbid atopic dermatitis or another atopic condition (i.e., the subject has a history of atopy.) In exemplary embodiments, the subject has co-morbid mild atopic dermatitis. In other embodiments, the subject has co-morbid moderate atopic dermatitis, moderate-to-severe atopic dermatitis, or severe atopic dermatitis.

In a further exemplary embodiment, a “subject in need thereof” is a subject who has mild PN, moderate PN, moderate-to-severe PN, or severe PN. In some embodiments, a subject with mild PN has an IGA PN-S score of 2 or mild. In some embodiments, a subject with moderate PN has an IGA PN-S score of 3 or moderate. In some embodiments, a subject with severe PN has an IGA PN-S score of 4 or severe. In some embodiments, a subject with moderate-to-severe PN has an IGA PN-S score of 3 or 4 (moderate or severe). In some embodiments, a subject with moderate-to-severe PN has a minimum of 20 PN nodules in total on both legs, and/or both arms and/or trunk.

In other exemplary embodiments, a “subject in need thereof” may be a subject who has uncontrolled PN. In some embodiments, a subject with uncontrolled PN failed treatment with topical therapies. In some embodiments, a subject with uncontrolled PN has severe itch. In some embodiments, a subject with uncontrolled PN has more than 20 PN nodules.

In other exemplary embodiments, a “subject in need thereof” may be a subject who has severe itch.

### Methods for Assessing Pharmacodynamic PN-Associated Parameters

Methods for assessing one or more pharmacodynamic PN-associated parameters in a subject in need thereof, caused by administration of a pharmaceutical composition comprising an IL-4R antagonist, are provided. A reduction in the incidence of PN symptoms or an improvement in a PN-associated PRO or ClinRO measure may correlate with an improvement in one or more pharmacodynamic PN-associated parameters; however, such a correlation is not necessarily observed in all cases.

Examples of “pharmacodynamic PN-associated parameters” include, for example, the following: (a) biomarker expression levels and (b) serum protein and RNA analysis. An “improvement in a pharmacodynamic PN-associated parameter” means, for example, a decrease from baseline of one or more biomarkers, such as IgE, eosinophil level, c-reactive protein (CRP), IL-6, D-dimer, medium platelet volume (MPV), IL-17, IL-18, IL-31, IL-33, and metalloproteinase-9. As used herein, the term “baseline,” with regard to a pharmacodynamic PN-associated parameter, means the numerical value of the pharmacodynamic PN-associated parameter for a patient prior to or at the time of administration of a pharmaceutical composition described herein.

To assess a pharmacodynamic PN-associated parameter, the parameter is quantified at baseline and at a time point after administration of the pharmaceutical composition. For example, a pharmacodynamic PN-associated parameter may be measured at about day 1, about day 2, about day 3, day 4, about day 5, about day 6, about day 7, about day 8, about day 9, about day 10, about day 11, about day 12, about day 14, or at about week 3, about week 4, about week 5, about week 6, about week 7, about week 8, about week 9, about week 10, about week 11, about week 12, about week 13, about week 14, about week 15, about week 16, about week 17, about week 18, about week 19, about week 20, about week 21, about week 22, about week 23, about week 24, or longer, after the initial treatment with the pharmaceutical composition. The difference between the value of the parameter at a particular time point following initiation of treatment and the value of the parameter at baseline is used to establish whether there has been change, such as an “improvement,” in the pharmacodynamic PN-associated parameter (e.g., an increase or decrease, as the case may be, depending on the specific parameter being measured).

In certain embodiments, administration of an IL-4R antagonist to a patient causes a change, such as a decrease or increase, in expression of a particular biomarker. PN-associated biomarkers include, but are not limited to total IgE, c-reactive protein (CRP), IL-6, D-dimer, medium platelet volume (MPV), IL-17, IL-18, IL-31, IL-33, and metalloproteinase-9. For example, administration of an IL-4R antagonist to a PN patient can cause a decrease in total serum IgE levels. The decrease can be detected at about week 1, about week 2, about week 3, about week 4, about week 5, or longer following administration of the IL-4R antagonist. Biomarker expression can be assayed by methods known in the art. For example, protein levels can be measured by ELISA (Enzyme Linked Immunosorbent Assay). RNA levels can be measured, for example, by reverse transcription coupled to polymerase chain reaction (RT-PCR).

Biomarker expression, as discussed above, can be assayed by detection of protein or RNA in serum. The serum samples can also be used to monitor additional protein or RNA biomarkers related to response to treatment with an IL-4R antagonist or IL-4/IL-13 signaling (e.g., by measuring soluble IL-4Rα, IL-4, IL-13, etc.). In some embodiments, RNA samples are used to determine RNA levels (non-genetic analysis), e.g., RNA levels of biomarkers; and in other embodiments, RNA samples are used for transcriptome sequencing (e.g., genetic analysis).

### Formulations

In some embodiments, the antibody or antigen binding fragment thereof is formulated in a composition comprising: i) about 150 mg/mL of antibody or an antigen-binding fragment thereof that specifically binds to IL-4R, ii) about 20 mM histidine, iii) about 12.5 mM acetate, iv) about 5% (w/v) sucrose, v) about 25 mM arginine hydrochloride, vi) about 0.2% (w/v) polysorbate 80, wherein the pH of the formulation is about 5.9, and wherein the viscosity of the formulation is about 8.5 cPoise.

In alternative embodiments, the antibody or antigen binding fragment thereof is formulated in a composition comprising: i) about 175 mg/mL of antibody or an antigen-binding fragment thereof that specifically binds to IL-4R, ii) about 20 mM histidine, iii) about 12.5 mM acetate, iv) about 5% (w/v) sucrose, v) about 50 mM arginine hydrochloride, and vi) about 0.2% (w/v) polysorbate 80, wherein the pH of the formulation is about 5.9, and wherein the viscosity of the formulation is about 8.5 cPoise.

In specific embodiments, the antibody or antigen-binding fragment thereof comprises an HCVR comprising the amino acid sequence of SEQ ID NO: 1 and an LCVR comprising the amino acid sequence of SEQ ID NO: 2.

In specific embodiments, the antibody comprises dupilumab. Unless otherwise specified, the term “dupilumab” also includes any biosimilars thereof.

Suitable stabilized formulations are also set forth in U.S. Pat. No. 8,945,559, which is incorporated herein by reference in its entirety for all purposes.

The present disclosure is further illustrated by the following example which should not be construed as further limiting. The contents of the figures, tables and all references, patents and published patent applications cited throughout this application are expressly incorporated herein by reference for all purposes.

Furthermore, in accordance with the present disclosure there may be employed conventional molecular biology, microbiology, and recombinant DNA techniques within the skill of the art. Such techniques are explained fully in the literature. See, e.g., Green & Sambrook, Molecular Cloning: A Laboratory Manual, Fourth Edition (2012) Cold Spring Harbor Laboratory Press, Cold Spring Harbor, N.Y.; DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

### EXAMPLES

The following examples are put forth so as to provide those of ordinary skill in the art with a complete disclosure and description of how to make and use the methods and compositions featured in the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The exemplary IL-4R antagonist used in the following Example is the human anti-IL-4R antibody named dupilumab (also referred to herein as “mAb1” or DUPIXENT®).

### Example 1. A Randomized, Double Blind, Placebo-Controlled, Multi-Center, Parallel Group Study to Evaluate the Efficacy and Safety of Dupilumab in Patients with Prurigo Nodularis Who are Inadequately Controlled on Topical Prescription Therapies or when Those Therapies are not Advisable (2 Phase3 Studies of Similar Design and Population—PRIME & PRIME2)

### Objectives

### Primary Objective:

To demonstrate the efficacy of dupilumab on itch response in patients with PN, inadequately controlled on topical prescription therapies or when those therapies are not advisable.

### Secondary Objectives:

To demonstrate the efficacy of dupilumab on additional itch endpoints in patients with PN, inadequately controlled on topical prescription therapies or when those therapies are not advisable.

To demonstrate efficacy of dupilumab on skin lesions of PN.

To demonstrate the improvement in health-related quality of life (HRQoL).

To evaluate safety outcome measures.

To evaluate immunogenicity of dupilumab.

### Endpoints

### Primary Endpoint for PRIME

Proportion of participants with improvement (reduction) in worst-itch numeric rating scale (WI-NRS) by ≥4 from baseline to week 24.

### Primary Endpoint for PRIME2:

Proportion of participants with improvement (reduction) in worst-itch numeric rating scale (WI-NRS) by ≥4 from baseline to week 12.

### Secondary Endpoints:

Proportion of participants with improvement (reduction) in WI-NRS by ≥4 from baseline to Week 24 (PRIME2 study.)

Proportion of participants with both an improvement (reduction) in WI-NRS by ≥4 from baseline to Week 24 and an IGA PN-S 0 or 1 score at Week 24. This endpoint captures the proportion of participants with concomitant improvement (reduction) in WI-NRS and an IGA PN-S score of 0 or 1 on the same day.

Time to onset of effect on pruritus as measured by proportion of participants with an improvement (reduction) in WI-NRS by ≥4 from baseline during the 24-week treatment period.

Change from baseline in WI-NRS at Week 24.

Change from baseline in WI-NRS at Week 12.

Percent change from baseline in WI-NRS at Week 24.

Percent change from baseline in WI-NRS at Week 12.

Percent change from baseline in WI-NRS at Week 4.

Percent change from baseline in WI-NRS at Week 2.

Percent change from baseline in WI-NRS over time until Week 24.

Proportion of participants with WI-NRS reduction ≥4 at Week 4.

Proportion of participants with WI-NRS reduction ≥4 over time until Week 24.

Onset of action in change from baseline in WI-NRS (first p<0.05 difference from placebo in the daily WI-NRS that remains significant at subsequent measurements) until Week 12.

Proportion of participants with Investigator's Global Assessment 0 or 1 score for PN-Stage (IGA PN-S) at Week 24.

Proportion of participants with IGA PN-S 0 or 1 score at Week 12.

Proportion of participants with IGA PN-S 0 or 1 score at Week 8.

Proportion of participants with IGA PN-S 0 or 1 score at Week 4.

Change from baseline in IGA PN-S score at Week 24.

Change from baseline in IGA PN-S score at Week 12.

Change from baseline in IGA PN-S score at Week 8.

Change from baseline in IGA PN-S score at Week 4.

Proportion of participants with Investigator's Global Assessment 0 or 1 score for PN-Activity (IGA PN-A) at Week 24.

Proportion of participants with IGA PN-A 0 or 1 score at Week 12.

Proportion of participants with IGA PN-A 0 or 1 score at Week 8.

Proportion of participants with IGA PN-A 0 or 1 score at Week 4.

Change from baseline in HRQoL, as measured by Dermatology Life Quality Index (DLQI) to Week 24.

Change from baseline in HRQoL, as measured by DLQI to Week 12.

Percentage of participants experiencing treatment-emergent adverse events (TEAEs) or serious adverse events (SAEs) from baseline through Week 24.

Incidence of treatment-emergent antidrug antibodies (ADA) against dupilumab over time.

### Study Design

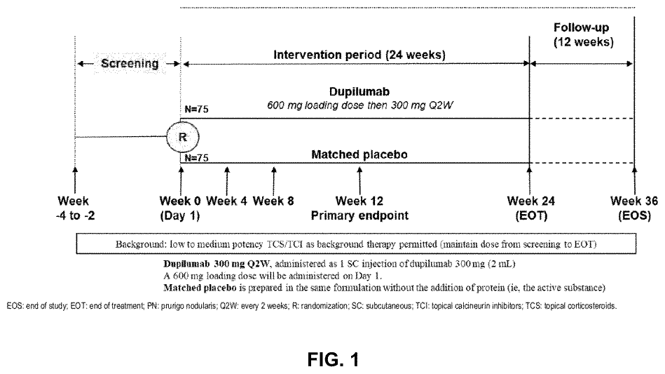

This study was a multi-center, 24-week treatment, parallel, double-blind, randomized, placebo-controlled study to evaluate the use of dupilumab in patients with PN inadequately controlled on topical prescription therapies or when those therapies are not advisable. The study assessed the effect of dupilumab on itch improvement as well as its effect on PN lesions, on patients' HRQoL, anxiety and depression, sleep quality and skin pain, and overall health status. As shown in FIG. 1, this was a parallel, treatment study, with 2 arms, that is blinded/masked for participants and investigators.

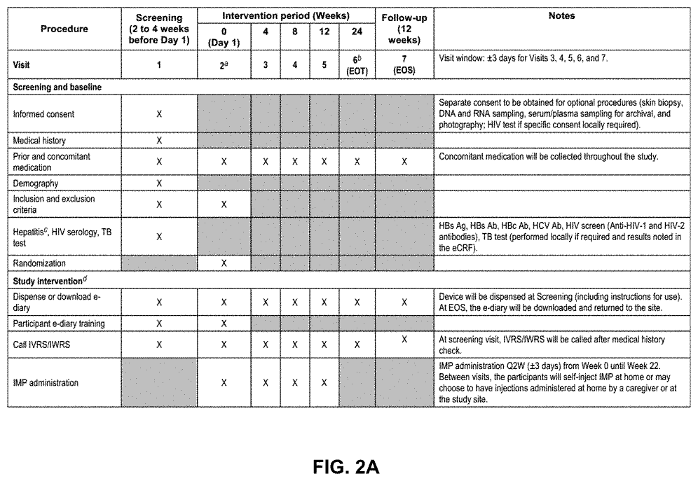

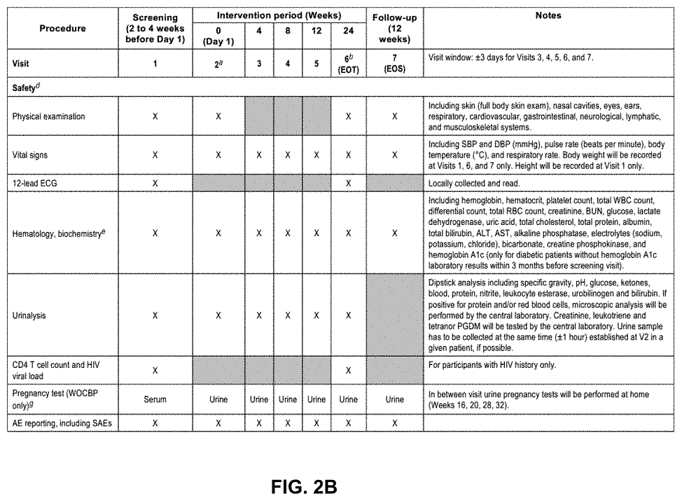

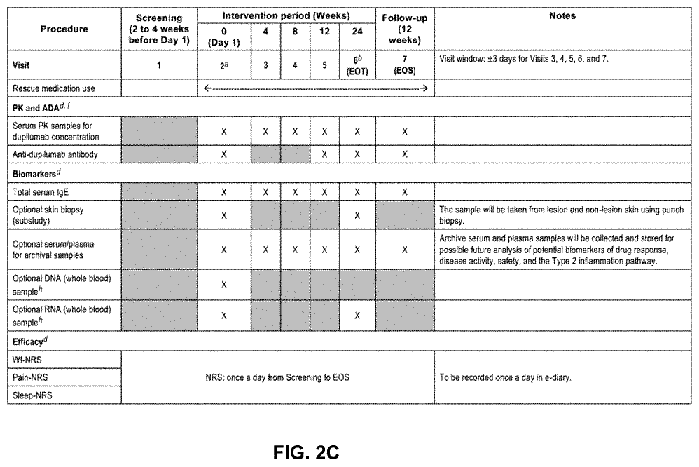

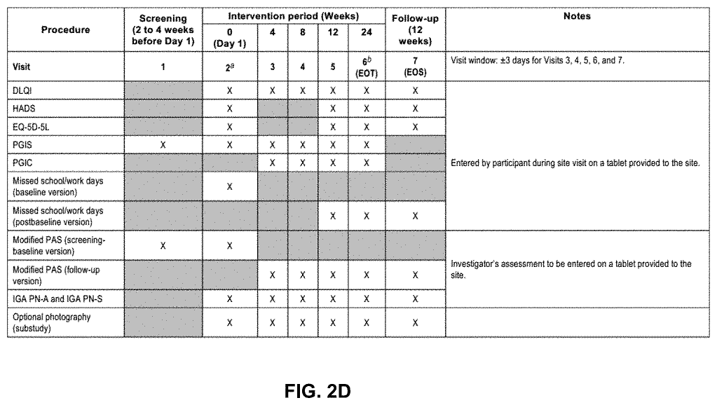

Approximately 150 participants were randomized 1:1. This corresponds to approximately 75 participants who were randomly assigned to each intervention arm. Participants who satisfied the inclusion and exclusion criteria were randomized (1:1) to one of the following investigational medicinal product (IMP) treatment groups: 300 mg Dupilumab and placebo. The study of activities is shown in FIG. 2A-D.

### Duration of Study Period (Per Participant)

Screening period (2-4 weeks); Randomized IMP intervention period (24 weeks); and Follow-up period (12 weeks)

### Study Interventions

### Investigational Medicinal Product:

Dupilumab 300 mg and placebo matching dupilumab 300 mg supplied in prefilled syringes that are visually indistinguishable.

### Dupilumab:

Formulation: dupilumab 300 mg: a 150 mg/mL dupilumab solution in a pre-filled syringe to deliver 300 mg in a 2 mL injection.

Route of administration: subcutaneous (SC) injection.

Dose regimen: 300 mg every 2 weeks (Q2W) after an initial loading dose of 600 mg (2 injections of 300 mg) on Day 1.

### Placebo:

Formulation: identical formulation to the active 300 mg formulation without dupilumab, in a pre-filled syringe to deliver placebo in a 2 mL injection.

Route of administration: SC injection.

Dose regimen: 1 injection Q2W after an initial loading dose (2 injections) on Day 1.

### Non-Investigational Medicinal Products

Participants were required to apply moisturizers (emollients) once or twice daily for at least 5 out of the 7 consecutive days immediately before day 1 and continue until week 36.

If participants were on a stable regimen of low to medium potency TCS or TCI at the screening visit, they could continue their topical steroid application once daily without tapering from screening to week 24. If specific lesions resolved, the participant could stop applying steroids to those sites but was permitted to continue applying to persistent lesions. If participants were on stable regimens of high potency or superpotent steroids, participants should decrease potency to medium potency TCS and continue to apply daily from screening to week 24. Occlusion was not allowed from screening to week 24.

Participants could be rescued with high potency or superpotent TCS/TCI as needed throughout the study.

### Inclusion Criteria

Participants were eligible to be included in the study only if all of the following criteria applied:

### Age

Participants must be 18 to 80 years of age, at the time of signing the informed consent.

### Type of Participant and Disease Characteristics

Patients with a clinical diagnosis of PN, as defined by all of the following:

Diagnosed by a dermatologist for at least 3 months before the screening visit.

On the WI-NRS ranging from 0 to 10, patients must have an average worst itch score of ≥7 in the 7 days prior to Day 1. (Baseline pruritus NRS average score for maximum itch intensity was determined based on the average of daily NRS scores for maximum intensity (the daily score ranges from 0 to 10) during the 7 days immediately preceding randomization. A minimum of 4 daily scores out of the 7 days is required to calculate the baseline average score. For patients who did not have at least 4 daily scores reported during the 7 days immediately preceding the planned randomization date, randomization should be postponed until this requirement is met, but without exceeding the 28-day maximum duration of the screening period.)

Patients needed to have a minimum of 20 PN lesions in total on both legs, and/or both arms and/or trunk, at screening visit and on Day 1. (Patients needed to have bilaterally symmetrical lesions on the extremities. The presence of lesions on at least 2 body surface areas is required.)

History of failing a 2-week course of medium-to-superpotent TCS or when TCS are not medically advisable. (Failure was defined as patients who are unable to achieve and/or maintain remission and low disease activity (similar to IGA PN-S score of ≤2 [≤19 nodules]) despite treatment with a daily regimen of medium-to-superpotent TCS (±TCI as appropriate), applied for at least 14 days, or for the maximum duration recommended by the product prescribing information, whichever is shorter.)

Have applied a stable dose of topical emollient (moisturizer) once or twice daily for at least 5 out of the 7 consecutive days immediately before day 1.

Participants must have been willing and able to complete a daily symptom eDiary for the duration of the study.

### Sex

Participants could be male or female. Contraceptive use by women was consistent with local regulations regarding the methods of contraception for those participating in clinical studies. A female participant was eligible to participate if she was not pregnant or breastfeeding, and at least one of the following conditions applies: not a WOCBP or a WOCBP and agreed to use a contraceptive method during the study (at a minimum until 12 weeks after the last dose of study intervention). A WOCBP must have had a negative highly sensitive pregnancy test (urine or serum as required by local regulations) on day 1 before the first dose of study intervention.

### Informed Consent

Capable of giving signed informed consent. In countries where legal age of majority is above 18 years, a specific ICF must also have been signed by the participant's legally authorized representative.

### Exclusion Criteria

Participants were excluded from the study if any of the following criteria apply:

### Medical Conditions

Presence of skin morbidities other than PN and mild AD that may interfere with the assessment of the study outcomes. Conditions including, but not limited to, the following: scabies, insect bite, lichen simplex chronicus, psoriasis, acne, folliculitis, habitual picking, lymphomatoid papulosis, chronic actinic dermatitis, dermatitis herpetiformis, sporotrichosis, and bullous disease. (NOTE: patients with mild active AD will represent up to 10% of the atopic PN study population.)

PN secondary to medications (e.g., opioids, angiotensin converting enzyme [ACE] inhibitors).

PN secondary to medical conditions such as neuropathy or psychiatric disease (e.g., notalgia paresthetica, brachioradial pruritus, neurotic excoriations, obsessive compulsive disorder, delusions of parasitosis, etc.).

Patients with a documented AD severity moderate to severe within 6 months before the screening visit, or documented diagnosis of moderate to severe AD from screening visit to randomization visit (e.g., IGA AD of 3 or 4, eczema area and severity index [EASI]≥16, scoring atopic dermatitis [SCORAD]≥25).

Severe concomitant illness(es) under poor control that, in the investigator's judgment, would have adversely affected the patient's participation in the study. Examples include, but are not limited to patients with life expectancy shorter than 1 year, patients with uncontrolled diabetes (hemoglobin A1c≥9% according to the laboratory results within 3 months before screening visit), patients with cardiovascular conditions (e.g., Class III or IV heart failure according to the New York Heart Association classification), hepato-biliary conditions (e.g., Child-Pugh Class B or C), neurological conditions (e.g., demyelinating diseases), active major autoimmune diseases (e.g., lupus, inflammatory bowel disease, rheumatoid arthritis, etc.), other severe endocrinological, gastrointestinal, metabolic, pulmonary, or lymphatic diseases.

Severe renal conditions (e.g., patients with uremia and/or on dialysis).

Participants with uncontrolled thyroid disease.

Patients with active TB or non-tuberculous mycobacterial infection, or a history of incompletely treated TB were excluded from the study unless it was well documented by a specialist that the participant had been adequately treated and could start treatment with dupilumab in the medical judgment of the investigator and/or infectious disease specialist. Tuberculosis testing was performed on a country-by-country basis, according to local guidelines if required by regulatory authorities or ethics boards.

Diagnosed active endoparasitic infections; suspected or high risk of endoparasitic infection, unless clinical and (if necessary) laboratory assessment ruled out active infection before randomization.

Active chronic or acute infection (except HIV infection) requiring treatment with systemic antibiotics, antivirals, antiprotozoals, or antifungals within 2 weeks before screening visit or during the screening period.

Known or suspected immunodeficiency, including history of invasive opportunistic infections (e.g., TB, histoplasmosis, listeriosis, coccidioidomycosis, pneumocystosis, aspergillosis) despite infection resolution, or otherwise recurrent infections of abnormal frequency or prolonged duration suggesting an immune-compromised status, as judged by the investigator.

Active malignancy or history of malignancy within 5 years before the baseline visit, except completely treated in situ carcinoma of the cervix, completely treated and resolved non-metastatic squamous or basal cell carcinoma of the skin.

History of systemic hypersensitivity or anaphylaxis to any biologic therapy, including any excipients.

Any other medical or psychological condition including relevant laboratory abnormalities at screening that, in the opinion of the investigator, suggested a new and/or insufficiently understood disease, presented an unreasonable risk to the study patient as a result of his/her participation in this clinical trial, may have made the patient's participation unreliable, or may have interfered with study assessments.

History of substance and/or alcohol abuse.

Planned major surgical procedure during the patient's participation in this study.

### Prior/Concomitant Therapy

Exposure to another systemic or topical investigative drug (monoclonal antibodies as well as small molecules) within a certain time period prior to Visit 1 (screening), as follows: an interval of less than 6 months or <5 PK half-lives for investigative monoclonal antibodies, whichever is longer, and an interval of less than 30 days or <5 PK half-lives, whichever is longer, for investigative small molecules.

Having used any of the following treatments within 4 weeks before the screening visit: systemic immunosuppressive/immunomodulating drugs (e.g., systemic corticosteroids, cyclosporine, mycophenolate-mofetil, interferon gamma, Janus kinase inhibitors, azathioprine, methotrexate, hydroxychloroquine, dapsone, sulfasalazine, colchicine, etc.); intralesional corticosteroid injections and cryotherapy; phototherapy, including tanning beds; naltrexone or other opioid antagonist; or gabapentin, pregabalin, and thalidomide.

Starting to use the following treatments or changed the dose of the following treatments in 3 months before the screening visit or expected the dose of the following treatments to be changed throughout the study: paroxetine, fluvoxamine, or other selective serotonin reuptake inhibitors (SSRIs); serotonin and norepinephrine reuptake inhibitors (SNRIs); or

amitriptyline or other tricyclic or tetracyclic antidepressants.

Previous treatment with biologic medicines within the following timeframe: any cell-depleting agents including but not limited to rituximab: within 6 months before the screening visit; omalizumab: within 5 months before screening visit; or other immunomodulatory biologics: within 5 half-lives (if known) or 16 weeks before the screening visit, whichever is longer.

Initiation of treatment with prescription moisturizers or moisturizers containing additives such as ceramide, hyaluronic acid, urea, menthol, polidocanol, or filaggrin degradation products during the screening period (patients could continue using stable doses of such moisturizers if initiated before the screening visit).

Initiation of treatment with TCS/TCI (any potency) during the screening period or treatment with high potency or superpotent TCS/TCI during the screening period.

For participants who were on a stable regimen of TCS/TCI (maintain same medicine, same dose from 2 weeks prior to screening visit) at the screening visit: application of TCS/TCI on fewer than 6 days during the 7 days immediately preceding randomization or application of TCS/TCI of incorrect potency within 7 days before Day 1.

Treatment with a live (attenuated) vaccine within 4 weeks before the screening visit. (NOTE: For patients who have vaccination with live, attenuated vaccines planned during the course of the study (based on national vaccination schedule/local guidelines), it was determined, after consultation with a physician, whether the administration of vaccine could be postponed until after the end of study, or preponed to before the start of the study, without compromising the health of the patient: patient for whom administration of live (attenuated) vaccine can be safely postponed would be eligible to enroll into the study or Patients who had their vaccination preponed could enroll in the study only after a gap of 4 weeks following administration of the vaccine.)

Planned or anticipated use of any prohibited medications and procedures during screening and study treatment period.

### Prior/Concurrent Clinical Study Experience

Participation in prior dupilumab clinical study; treated in the past with dupilumab; prior use of biologics for PN.

### Diagnostic Assessments

For participants without history of HIV infection before screening visit, positive HIV serology at screening.

For participants with history of HIV infection with CD4+ counts ≤300 cells/4 and/or detectable HIV viral load at screening.

Participants with any of the following result at screening: positive (or indeterminate) HBs Ag, positive total HBc Ab confirmed by positive HBV DNA, or positive HCV Ab confirmed by positive HCV RNA.

### Other Exclusions

Individuals accommodated in an institution because of regulatory or legal order; prisoners or subjects who are legally institutionalized.

Any country-related specific regulation that would have prevented the subject from entering the study.

Participant not suitable for participation, whatever the reason, as judged by the investigator, including medical or clinical conditions, or participants potentially at risk of noncompliance to study procedures.

Participants are employees of the clinical study site or other individuals directly involved in the conduct of the study, or immediate family members of such individuals.

Participants are employees of the clinical study site or other individuals directly involved in the conduct of the study, or immediate family members of such individuals.

Sensitivity to any of the study interventions, or components thereof, or drug or other allergy that, in the opinion of the Investigator, contraindicates participation in the study.

### Study Intervention

Study intervention was defined as any investigational intervention(s), marketed product(s), placebo, or medical device(s) intended to be administered to a study participant according to the study protocol. An overview of the study interventions administered in presented in Table 7 below.

TABLE 7
Overview of study interventions administered

| ARM name | Dupilumab | Placebo |
|---|---|---|
| Intervention name | Dupilumab 300 mg | Placebo matching |
| | | dupilumab 300 mg |
| Type | Biological/Vaccine | Other |
| Dose formulation | A 150 mg/mL dupilumab | Identical formulation |
| | solution in a pre-filled | to the active 300 mg |
| | syringe to deliver | formulation without |
| | 300 mg in 2 mL | dupilumab, in a pre- |
| | | filled syringe to |
| | | deliver placebo in 2 mL |
| Unit dose | 300 mg | 0 mg (placebo) |
| strength(s) | | |
| Dosage level(s) | 300 mg every 14 ± 3 | 0 mg every 14 ± 3 |
| | days after an initial | days with a loading |
| | loading dose of 600 mg | dose of 0 mg |
| Route of | Subcutaneous^{a} | Subcutaneous^{a} |
| administration | | |
| IMP and NIMP | IMP | IMP |
| Packaging and | One glass pre-filled | One glass pre-filled |
| labeling | syringe packed in a | syringe packed in a |
| | patient kit box. Both the | patient kit box. Both |
| | glass pre-filled syringe | glass pre-filled the |
| | and the box will be | syringe and the box |
| | labeled as required per | will be labeled as |
| | country requirement. | required per |
| | | country requirement |

^{a}Subcutaneous injection sites should alternate between the upper thighs, 4 quadrants of the abdomen or the upper arms, so that the same site is not injected twice during consecutive administrations. Injection in the upper arms can only be done by a trained person (parent/legally authorized representative/caregiver trained by Investigator or Delegate) or health care professional but not the participant themselves.
IMP: investigational medicinal product; NIMP: noninvestigational medicinal product.

The Investigator or delegate trained the patient (or caregiver) how to prepare and inject IMP at Visit 2. He/she injected the first of the two injections. The participant (or caregiver) performed the second injection under the supervision of the Investigator or delegate. The patient was also trained by the site staff to recognize potential signs and symptoms of hypersensitivity reaction in order to self-monitor at home for at least 30 minutes (or longer per country specific or local site-specific requirements) following injection. In case of hypersensitivity symptoms the patient were advised to contact healthcare provider/emergency.

When the participant had a study visit, the IMP was administered following clinical procedures and blood collection. Patients were monitored for at least 30 minutes.

Between the protocol-scheduled on-site visits, participants were allowed to self-inject IMP at home. Participants who preferred to have a healthcare professional administer the IMP could choose to have injections administered at home by a nurse or at the study site.

### Non-Investigational Medicinal Products

Starting from the screening visit, participants were instructed to use their daily moisturizer, if it did not contain any compound with known anti-itch effect (such as menthol, polidocanol, pramoxine, lidocaine, prilocaine, capsaicin, naltrexone, N-palmitoylethanolamine, etc.). It was not authorized to change emollients or moisturizers or applying products for itching relief during the course of the study.

Participants were required to apply moisturizers (emollients) once or twice daily for at least 5 out of the 7 consecutive days immediately before Day 1 and continue until week 36. All types of moisturizers were permitted, but patients could not initiate new treatment with prescription moisturizers or over-the-counter moisturizers containing additives during the screening period or during the intervention period. Patients could continue using stable doses of such moisturizers if initiated before the screening visit.

If participants were on a stable regimen of low to medium potency TCS or TCI at the screening visit, they could continue their topical steroid application once daily without tapering from screening to week 24. If specific lesions resolved, the participant could stop applying steroids to those sites but was permitted to continue applying to persistent lesions. If participants were on stable regimens of high potency or superpotent steroids, participants should have decreased potency to medium potency TCS and continued to apply daily from screening to week 24. A stable regimen for TCS was maintaining the same medicine (low to medium potency TCS), and maintaining the same frequency of treatment (once or twice daily) used from 2 weeks prior to screening. A stable regimen for TCI was maintaining the same medicine of TCI and the treatment frequency (once or twice daily) used from 2 weeks prior to screening. If participant's prior regimen was applying once daily, the participant would maintain daily application during study and for participants who had twice daily prior to screening, participation would maintain twice daily regimen during study. If specific lesions resolved, the participant could stop applying steroids to those sites but was permitted to continue applying to persistent lesions. Occlusion was not allowed from screening to week 24.

It was recommended that patients use triamcinolone acetonide 0.1% cream or fluocinolone acetonide 0.025% ointment for medium potency, and hydrocortisone 1% cream for low potency. If rescue with TCS was needed, it was recommended that patients use either betamethasone dipropionate 0.05% optimized ointment for high potency TCS or clobetasol propionate 0.05% cream for super high potency TCS. If patients had tolerance issues with any of these steroids or if they were not commercially available in some countries, they could substitute with products of the same potency from the list provided by the sponsor.

On areas treated with TCS, moisturizers must have been applied once daily only at the time when TCS was not applied (i.e., moisturizers and TCS should not have been used on the same areas at the same time during the day). For example, if TCS was applied in the evening, moisturizers were not be used in the evening on areas treated with TCS, but were applied to those areas in the morning. On areas not treated with TCS, moisturizers were applied twice daily (morning and evening.)

### Storage and Handling

The Investigator or designee had to confirm appropriate temperature conditions had been maintained during transit for all study intervention received and any discrepancies were reported and resolved before use of the study intervention.

Only participants enrolled in the study could receive study intervention and only authorized site staff could supply or administer study intervention. All study intervention must have been stored in a secure, environmentally controlled, and monitored (manual or automated) area in accordance with the labeled storage conditions with access limited to the Investigator and authorized site staff.

The Investigator, institution, or the head of the medical institution (where applicable) was responsible for study intervention accountability, reconciliation, and record maintenance (i.e., receipt, reconciliation, and final disposition records.)

### Randomization and Blinding

All participants were centrally assigned to randomized study intervention using an interactive response technology (IRT). Participants were randomized in 1:1 ratio to treatment arms described in Table 7.

Randomization was stratified by the following factors: documented history of atopy (atopic or non-atopic) (atopic: patients with a physician-documented history of atopic comorbidities defined as AD, allergic rhinitis/rhinoconjunctivitis, asthma, or food allergy, or a current diagnosis of at least one of these atopic comorbidities, per investigator judgement and non-atopic: patients without a physician-documented history of atopic comorbidities defined as AD, allergic rhinitis/rhinoconjunctivitis, asthma or food allergy, and without a current diagnosis of at least one of such atopic comorbidities, per investigator judgement); stable use of TCS/TCI (yes or no); and country/territory code.

A randomized participant was defined as a participant who was allocated to a randomized intervention regardless whether the intervention kit was used or not (i.e., participant registered by the IRT). A participant could not be randomized more than once in the study.

### Methods of Blinding

Dupilumab 300 mg and placebo matching dupilumab 300 mg were provided in identically matched 2 mL pre-filled syringes that were visually indistinguishable. Syringes and box were labeled with a treatment kit number.

### Study Intervention Compliance

Investigator or his/her delegate had to ensure that IMP was administered to each participant according to the labeling instructions.

Intervention units were returned by the participant at each visit. The Investigator counted the number of remaining kit/pre-filled syringe, and filled in the IMP accountability and inventory forms. The Investigator or his/her delegate recorded the dosing information on the appropriate page(s) of the eCRF. Participant compliance with study intervention was assessed at each visit. Compliance was assessed by counting returned kit/pre-filled syringe. Deviation(s) from the prescribed dosage regimen was recorded in the eCRF.

### Concomitant Therapy

Any medication or vaccine (including over-the-counter or prescription medicines, vitamins, and/or herbal supplements) that the participant was receiving at the time of enrollment or received during the study had to be recorded along with: reason for use; dates of administration including start and end dates; and dosage information including dose and frequency.

The concomitant use of non-sedating antihistamine administration was allowed during the study except for treatment of AD or PN, but dose change of non-sedating antihistamine was not allowed both from week 11 to week 12 and from week 23 to week 24.

The concomitant use of the following therapies was prohibited during the entire study. Study treatment needed to be discontinued in participants receiving these treatments: systemic immunosuppressive/immunomodulating drugs (e.g., systemic corticosteroids, cyclosporine, mycophenolate-mofetil, interferon gamma, Janus kinase inhibitors, azathioprine, methotrexate, hydroxychloroquine, dapsone, sulfasalazine, colchicine, etc.); other monoclonal antibodies (which are biological modifiers); phototherapy, including tanning beds; naltrexone or other opioid antagonist; and gabapentin, pregabalin, and thalidomide.

The concomitant use of the following therapies was prohibited except if the dose had been stable for at least 3 months prior to screening, but study treatment did not need to be discontinued in participants receiving the following treatments: paroxetine, fluvoxamine, or other selective serotonin reuptake inhibitors (SSRIs); serotonin and norepinephrine reuptake inhibitors (SNRIs); and amitriptyline or other tricyclic or tetracyclic antidepressants. The dose needed to remain stable (can be reduced or discontinued if medically indicated), but should not be have been initiated or increased throughout the study.

The concomitant use of the following therapies was also prohibited during the entire study, but study treatment did not need to be discontinued in participants receiving the following treatments: intralesional corticosteroid injections and cryotherapy; sedating antihistamine; and non-sedating antihistamine used specifically for the treatment of itch secondary to AD or PN.

### Rescue Medicine

The following rescue medications could be used: dermatological preparations of high potency or superpotent TCS and TCI.

If medically necessary (i.e., to control intolerable PN symptoms), rescue treatment for PN could be provided to study patients at the discretion of the Investigator.

Although the use of rescue medications was allowed at any time during the study, the use of rescue medications should have been delayed, if possible, for at least 14 days following the initiation of the investigational treatment. The date and time of rescue medication administration as well as the name and dosage regimen of the rescue medication was recorded in the eCRF.

For the purpose of the efficacy responder analysis, a pre-specified algorithm was used to classify rescue (details in the SAP). In addition, a blinded review of all post-baseline medications to adjudicate rescue treatment, based on medical judgment, was performed to adjudicate rescue. Patients who received rescue treatment as per this adjudication during the study were considered treatment failures.

### Discontinuation of Study Intervention

In rare instances, it may have been necessary for a participant to permanently discontinue study intervention. If study intervention was permanently discontinued, the participant would remain in the study to be evaluated for safety.

The participants could withdraw from treatment with the IMP if he or she decided to do so, at any time and irrespective of the reason, or this may have been the investigator's decision. All efforts were made to document the reason(s) for treatment discontinuation and this should be documented in the eCRF.

Participants had to be permanently withdrawn from the study treatment for the following reasons: at their own request or at the request of their legally authorized representative (legally authorized representative means an individual or judicial or other body authorized under applicable law to consent on behalf of a prospective participant to the patient's participation in the procedure(s) involved in the research); if, in the investigator's opinion, continuation in the study would have been detrimental to the participant's well-being; at the specific request of the Sponsor; if they are treated with the specific prohibited medications; if they miss more than 2 consecutive IMP doses; in the event of a protocol deviation, at the discretion of the investigator or the Sponsor; any code broken at the requested of the investigator; pregnancy; anaphylactic reactions or systemic allergic reactions that are related to IMP and require treatment; diagnosis of a malignancy during study, excluding carcinoma in situ of the cervix, or squamous or basal cell carcinoma of the skin; any opportunistic infection or other infections whose nature or course may suggest an immunocompromised status; serum alanine aminotransferase (ALT)>3 upper limit of normal (ULN) and total bilirubin >2 ULN; or serum ALT >5 ULN if baseline ALT ≤2 ULN or ALT >8 ULN if baseline ALT >2 ULN.

### Efficacy Assessments

All screening evaluations had to be completed and reviewed to confirm that potential participants met all eligibility criteria. The Investigator maintained a screening log to record details of all participants screened and to confirm eligibility or record reasons for screening failure, as applicable.

Patient-Reported Outcome questionnaires including NRS were completed by the participants before the consultation and/or clinical tests, in a quiet place. The questionnaires were completed by the participants themselves, independently from their physician, the study nurse or any other medical personnel and without any help from friends or relatives.

### Worst-Itch Numeric Rating Scale

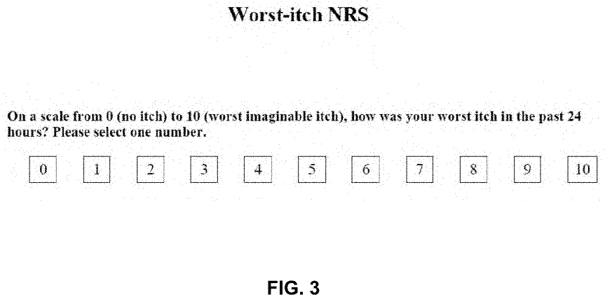

Worst-itch numerical rating scale (WI-NRS) is a PRO comprised of a single item rated on a scale from 0 (“No itch”) to 10 (“Worst imaginable itch”). Participants are asked to rate the intensity of their worst pruritus (itch) over the past 24 hours using this scale. The WI-NRS is shown in FIG. 3.

### Investigator's Global Assessment for Prurigo Nodularis

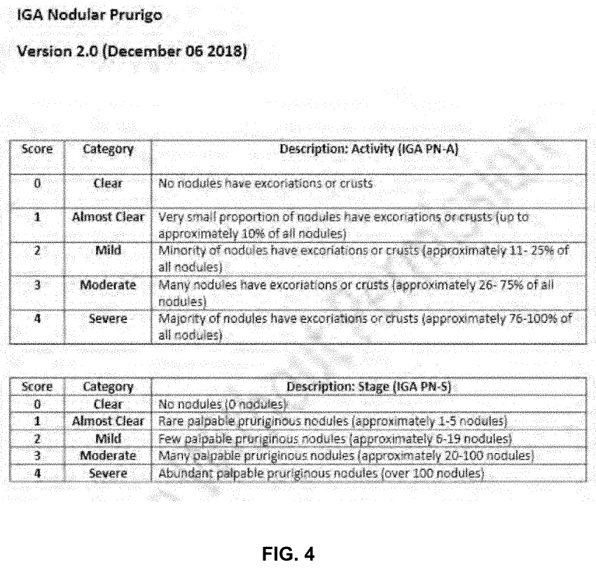

Investigator's global assessment for prurigo nodularis (IGA PN) is a clinician-reported outcome (ClinRO) that allows clinicians to assess the activity of PN (IGA PN-A) using a 5-point scale from 0 (clear) to 4 (severe); and the stage of the disease (IGA PN-S) using a 5-point scale from 0 (clear) to 4 (severe). The IGA PN is shown in FIG. 4.

### Prurigo Activity Score

The prurigo activity score (PAS) is a ClinRO measurement. The original PAS questionnaire Version 0.9 consists of 7 items, developed by expert clinicians in PN (Polking J, et al. Prurigo Activity Score (PAS): validity and reliability of a new instrument to monitor chronic prurigo. J Eur Acad Dermatol Venereol. 2018; 32(10):1754-60.) The items of the PAS evaluate the pruriginous lesions in terms of: type (visible lesions: Item 1a; predominant lesions: Item 1b); estimated number (Item 2); distribution (Item 3, 4); and size (biggest lesion: Item 6a; representative lesion: Item 6b).

Other items evaluate the representative body area and exact number of lesions (Item 5), the activity in terms of percentage of pruriginous lesions with excoriations/crusts on top (reflecting active scratching; Item 7a) and the percentage of healed pruriginous lesions (reflecting healing of chronic prurigo; Item 7b).

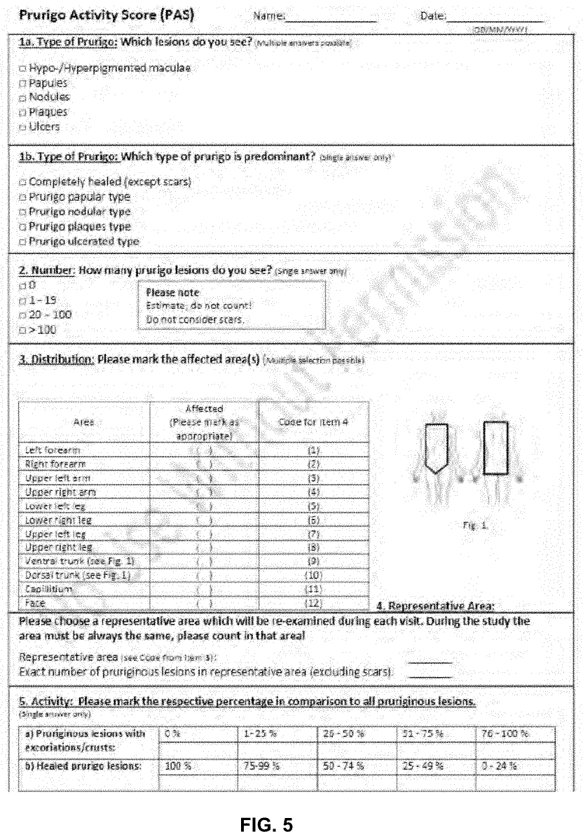

A 5-item simplified version of the PAS was used in the current study. In particular, Item 3 (lesion distribution) and Item 6 (lesion monitoring) of the original PAS were removed, and the response options were slightly modified and refined. This assessment tool is shown in FIG. 5. Clinicians completed the screening/baseline version at screening and baseline visits, and completed the follow-up version of the modified PAS at the other visits.

### Dermatology Life Quality Index

The dermatology life quality index (DLQI) is a PRO developed to measure dermatology-specific health-related quality of life (HRQoL) in adult patients (Finlay A Y, Khan G K. Dermatology Life Quality Index (DLQI)—a simple practical measure for routine clinical use. Clin Exp Dermatol. 1994; 19(3):210-6.) The instrument comprises 10 items assessing the impact of skin disease on patients' HRQoL over the previous week. The items cover symptoms, leisure activities, work/school or holiday time, personal relationships including intimate, the side effects of treatment, and emotional reactions to having a skin disease. It is a validated questionnaire used in clinical practice and clinical trials (Chemyshov P V. The evolution of quality of life assessment and use in dermatology. Dermatology. 2019; 235(3):167-74.) Response scale is a 4-point Likert scale (0=“not at all” and 3=“very much”) for nine items. The remaining one item about work/studying asks whether work/study has been prevented and then (if “No”) to what degree the skin condition has been a problem at work/study; the item is rated on a 3-point Likert scale (“Not at all” to “A lot”). Overall scoring ranges from 0 to 30, with a high score indicative of a poor HRQoL.

Efficacy data was collected via electronic devices. The e-diary was used for daily recording of participant's answers to the WI-NRS, pain-NRS, and sleep-NRS questionnaires. This device was dispensed at screening visit (Visit 1), including instructions for use. Participants were instructed on the use of the device. Recorded information was downloaded from this device daily. At end of study (EOS) Visit, the e-diary was downloaded and returned to the site.

Participants filled in the DLQI, HADS, EQ-5D-5L, PGIC, PGIS, and missed school/work days questionnaires during their site visit on a tablet that was provided to the site. This device was kept at the site during the study.

### Pain and Sleep Numeric Rating Scales

Participants were asked to rate their worst skin pain in the past 24 hours using a 0 to 10 numeric rating scale (NRS), with 0=No pain to 10=Worst pain possible.

In addition, participants were asked to rate their sleep quality on their past night upon awakening, using a 0 to 10 NRS, with 0=Worst possible sleep and 10=Best possible sleep.

Participants completed the skin pain NRS and sleep quality NRS once a day.

### Hospital Anxiety and Depression Scale

The hospital anxiety and depression scale (HADS) is a PRO instrument for screening anxiety and depression in non-psychiatric populations; repeated administration also provides information about changes to a patient's emotional state (Zigmond A S, Snaith R P. The hospital anxiety and depression scale. Acta Psychiatr. Scand. 1983; 67(6):361-70 and Herrmann C. International experiences with the Hospital Anxiety and Depression Scale—a review of validation data and clinical results. J Psychosom. Res. 1997; 42(1):17-41.) The HADS consists of 14 items, 7 each for anxiety and depression symptoms; possible scores range from 0 to 21 for each subscale. The following cut-off scores are recommended for both subscales: 0 to 7: normal; 8 to 10: borderline abnormal (borderline case); and 11 to 21: abnormal.

### Patient Global Impression of Change of Disease and Patient Global Impression of Severity

The patient global impression of change of disease (PGIC) is a one-item questionnaire that asks patients to provide the overall self-assessment of change in their PN overall on a 7-point scale, compared to just before patient started taking the study injection. Response choices are: 0=“very much better,” 1=“moderately better,” 2=“a little better,” 3=“no change,” 4=“A little worse,” 5=“moderately worse,” 6=“very much worse.”

The patient global impression of severity (PGIS) is a one-item questionnaire that asks patients to provide the overall self-assessment of their disease severity on a 4-point scale for the past week. Response choices are: 1=“none,” 2=“mild,” 3=“moderate,” 4=“severe.”

### EuroQol 5 Dimensions Questionnaire

The Euroqol-5 dimensions (EQ-5D) is a standardized PRO measure of health status developed by the EuroQol Group in order to provide a simple, generic measure of health for clinical and economic appraisal. (Herdman M, et al. Development and preliminary testing of the new five-level version of EQ-5D (EQ-5D-5L). Qual Life Res. 2011; 20(10):1727-36.) The EQ-5D consists of 2 parts: the descriptive system and the EQ visual analog scale (VAS). The EQ-5D descriptive system comprises the following 5 dimensions: mobility, self-care, usual activities, pain/discomfort, and anxiety/depression. Each dimension has 5 levels of perceived problems: “no problems,” “slight problems,” “moderate problems,” “severe problems” and “inability to do the activity.” The respondent is asked to indicate his/her health state by ticking (or placing a cross) in the box against the most appropriate statement in each of the 5 dimensions; this results in a 1-digit number expressing the level for that dimension. The digits for 5 dimensions can be combined in a 5-digit number describing the respondent's health state. The EQ VAS records the respondent's self-rated health on a vertical VAS where the endpoints are labeled “best imaginable health state (100)” and “worst imaginable health state (0).” This information can be used as a quantitative measure of health outcome as judged by the individual respondents.

### Missed School/Work Days

Participants who are employed or enrolled in school were asked to report the number of sick leave/missed school days since the last study assessment.

### Photography

Participants in selected sites who decide to participate in this sub-study need to provide separate consent. One or several lesions were photographed at baseline. The same lesions were photographed at subsequent visits to evaluate their progression.

### Safety Assessments

### Physical Examinations

A complete physical examination included, at a minimum, assessments of the skin (full body skin exam), nasal cavities, eyes, ears, respiratory, cardiovascular, gastrointestinal, neurological, lymphatic, and musculoskeletal systems. Investigators paid special attention to clinical signs related to previous serious illnesses. Any new finding or worsening of previous finding were reported as a new AE.

### Vital Signs

Vital signs were measured in a semi-supine or sitting position after 5 minutes rest and included body temperature, SBP and DBP, and pulse and respiratory rate. Blood pressure and pulse measurements were assessed using the same arm with a completely automated device. Manual techniques were used only if an automated device is not available. Body weight (kg) was measured at screening (Visit 1), EOT, and EOS visits. Height was measured at screening visit (Visit 1). Height and weight were measured with indoor clothing but without shoes.

### Electrocardiograms

Single 12-lead ECG was obtained using an ECG machine that automatically calculates the heart rate and measures PR, QRS, QT, and QTcF intervals. The ECG was recorded after 10 minutes of rest in the supine position.

### Results for PRIME/EFC16459

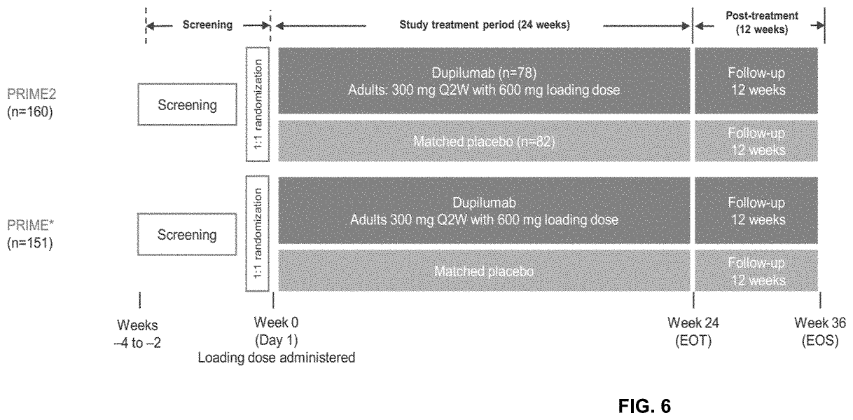

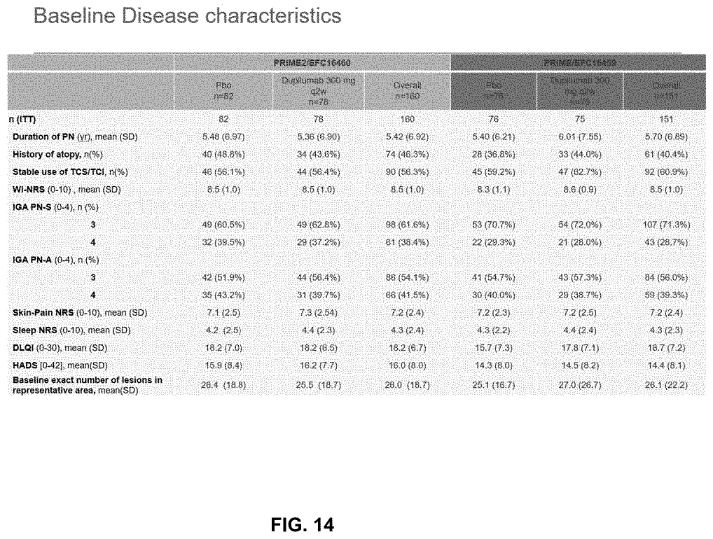

As shown in FIG. 6, PRIME/EFC16459 is one of two phase 3 studies. PRIME was a global trial with N=151 patients, 33%/50 from Asia, 27%/41 from Latin America, 25%/38 from Western Countries, 15%/22 from East Europe. The baseline disease characteristics for patients in the PRIME/EFC16459 study are shown in FIG. 14. The total enrolled patients had a mean (SD) WI-NRS of 8.5 (1.0) at baseline.

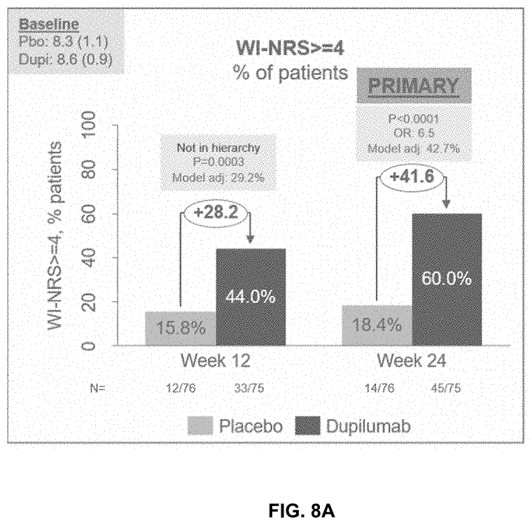

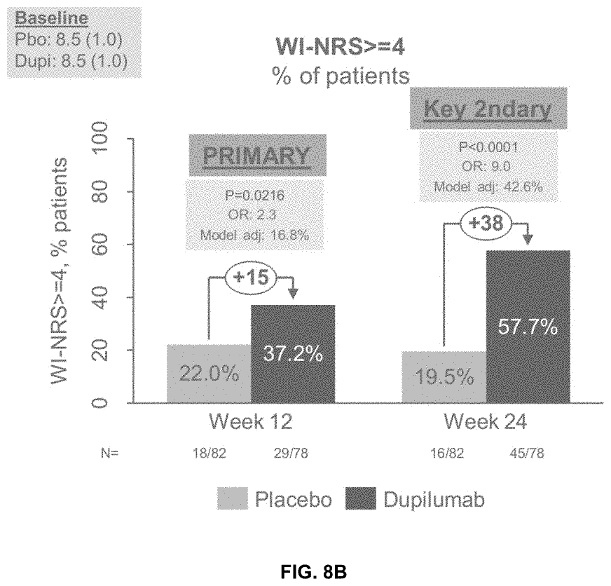

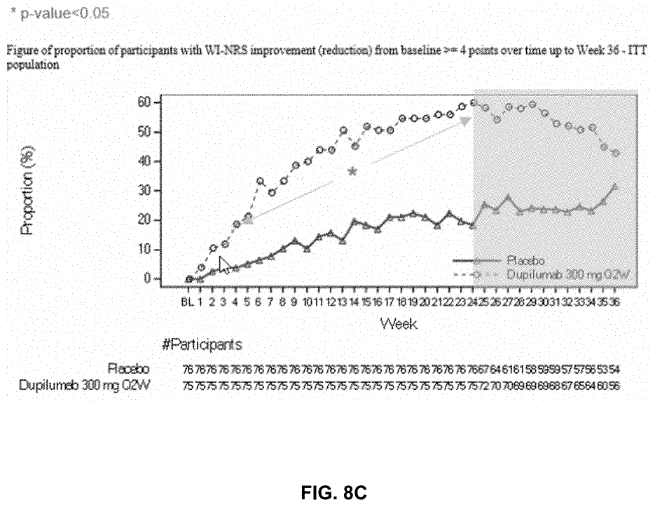

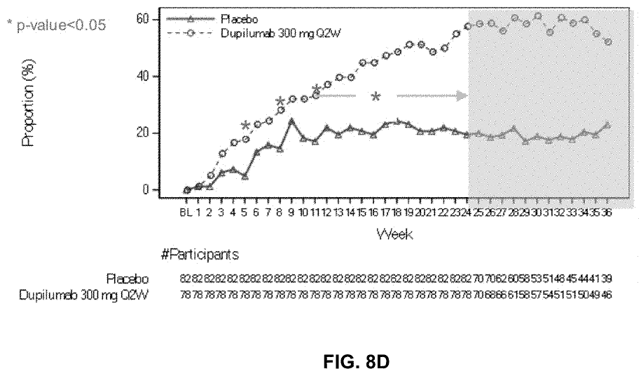

The primary endpoint was met with clinical and statistical significance. As shown in FIG. 8A, the proportion of participants who reached >4-point reduction of WI-NRS (0-10) at week 24 with dupilumab was 45 (60.0%) and with placebo was 14 (18.4%), p<0.0001. Thus, more than three times as many dupilumab treated participants experienced a clinically meaningful reduction in itch from baseline.

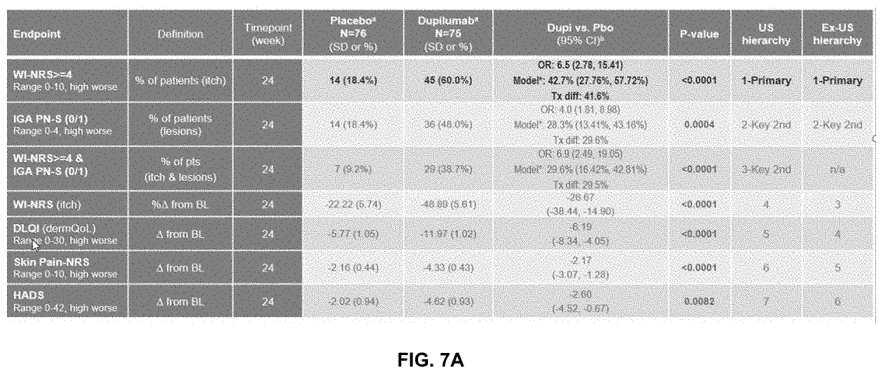

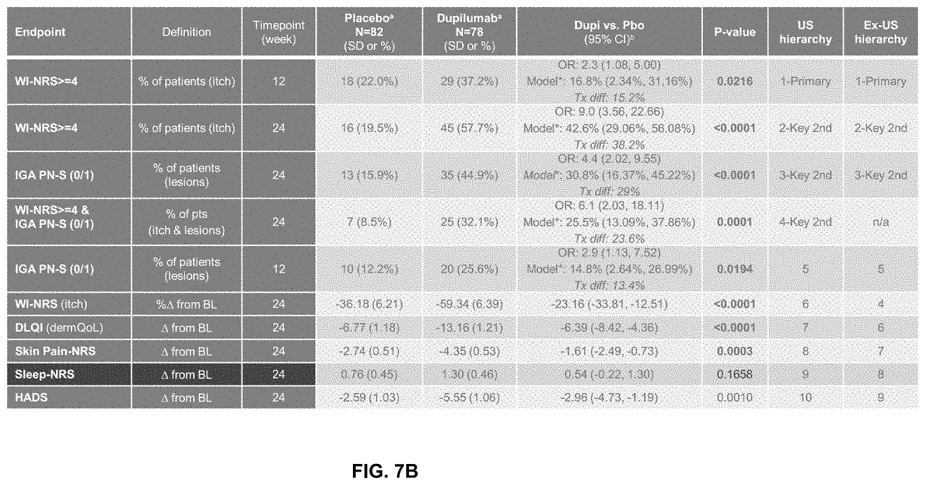

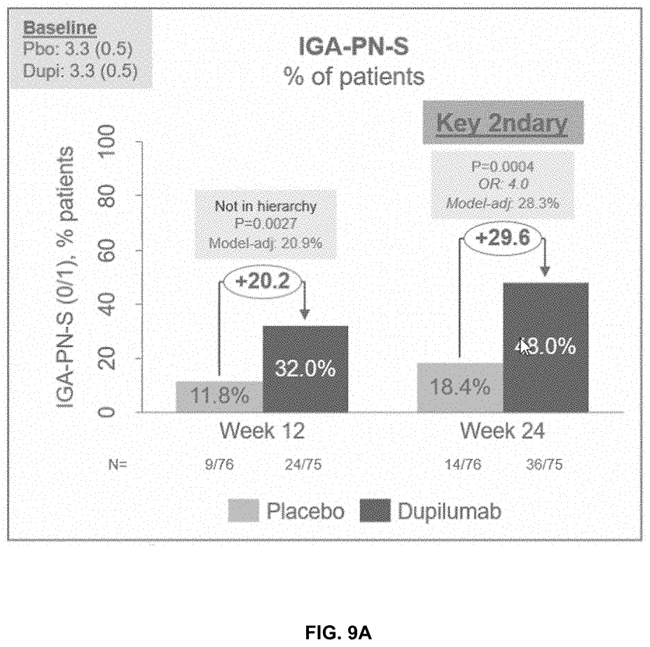

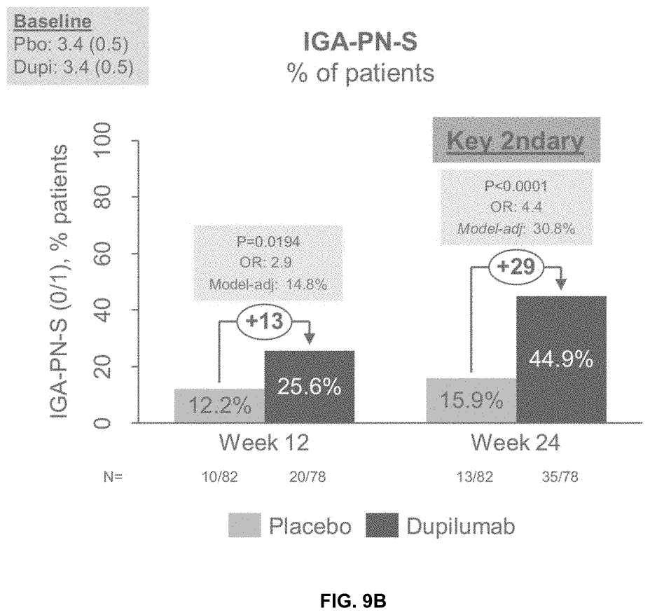

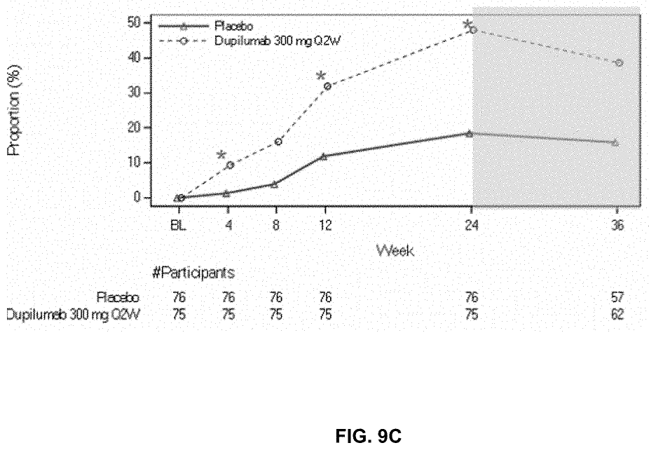

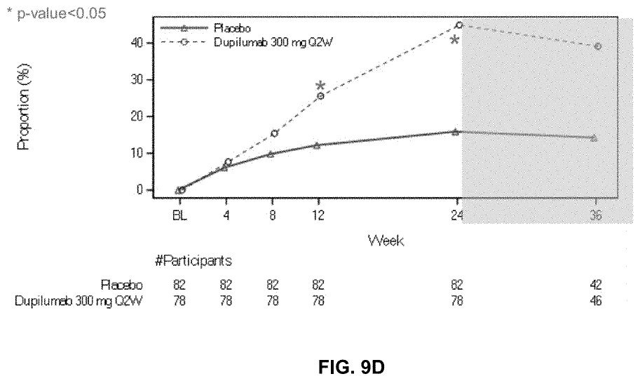

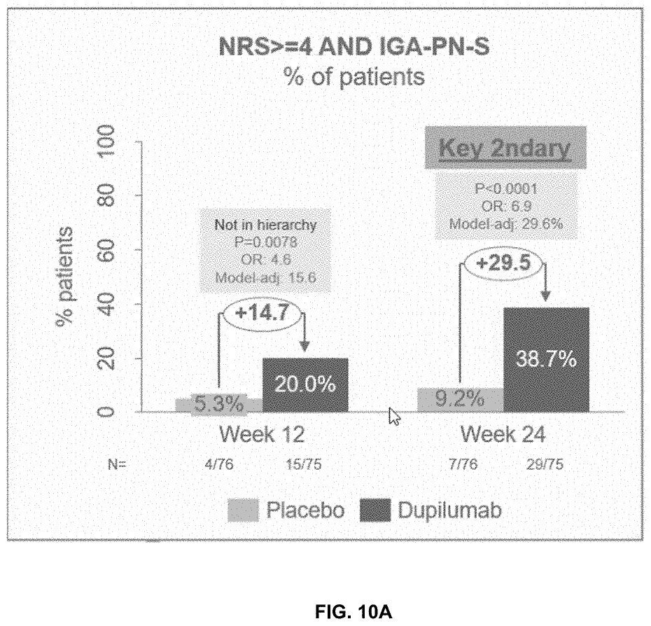

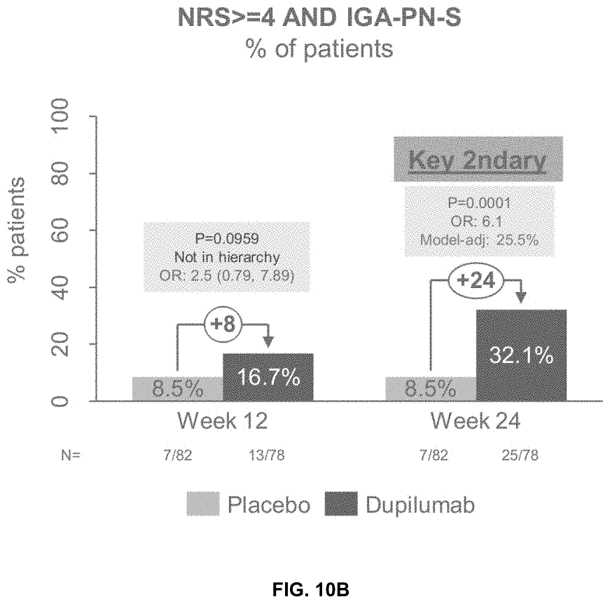

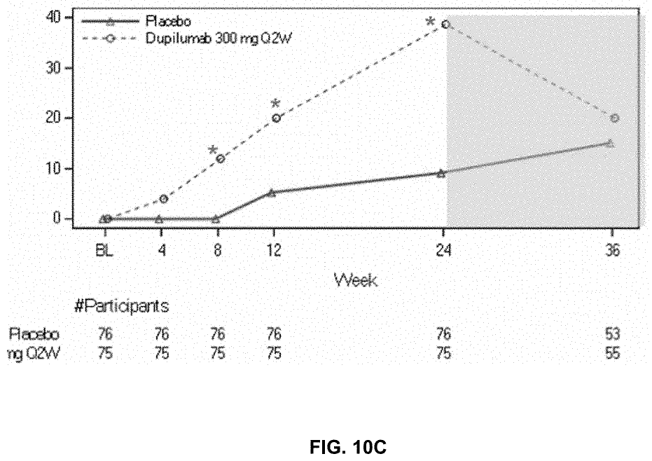

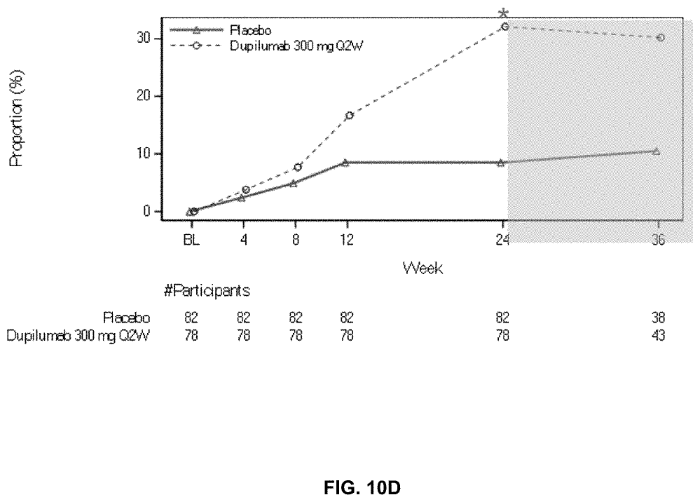

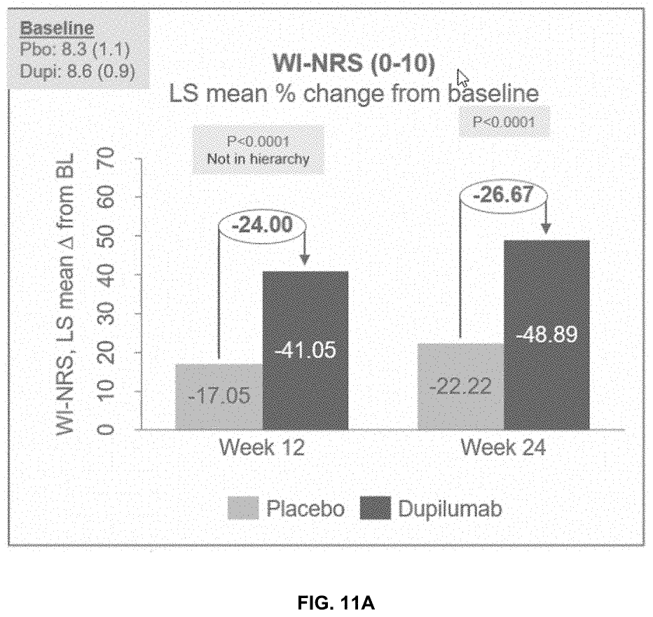

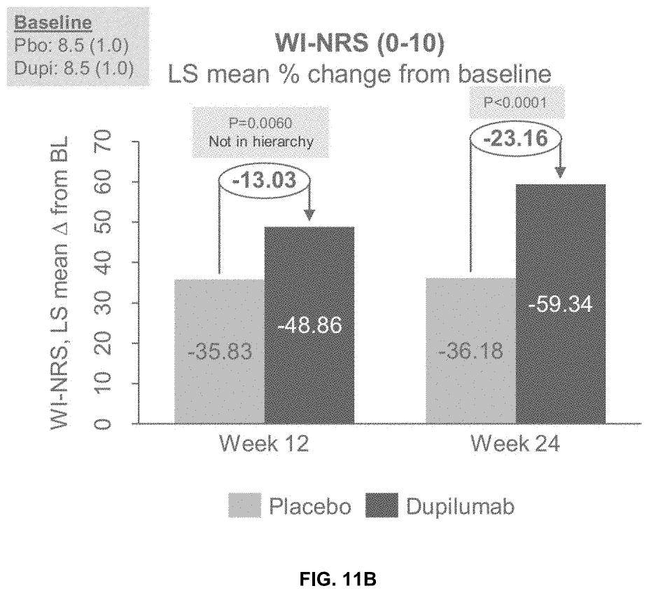

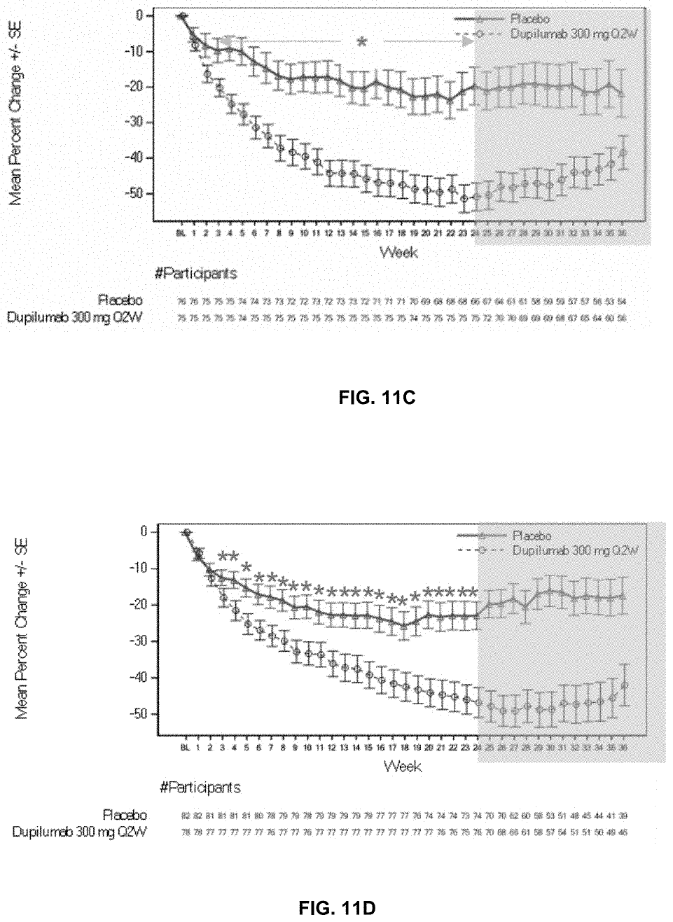

As shown in FIG. 7A, the primary endpoint and all multiplicity adjusted secondary endpoints were met with statistical significance including WI-NRS≥4, IGA PN-S score of 0 or 1, WI-NRS≥4 and IGA PN-S score of 0 or 1, WI-NRS (itch) percent change from baseline (FIG. 11A), DLQI change from baseline, skin pain-NRS change from baseline, and HADS (all at 24 weeks.) As compared to PRIME2, PRIME did not include 12-week endpoints or sleep-NRS in the hierarchy. As shown in FIG. 9A, the proportion of participants who reached an IGA PN-S score of 0 or 1 at week 24 with dupilumab was 48.0% and with placebo was 18.4%, (p=0.0004). Thus, nearly three times as many dupilumab treated participants achieved clear or almost clear skin at week 24, a key secondary endpoint. As shown in FIG. 10A, the proportion of participants with concomitant improvement (reduction) in WI-NRS by ≥4 from baseline to week 24 and an IGA PN-S score of 0 or 1 at week 24 with dupilumab was 38.7% and with placebo was 9.2%, (p<0.0001).

The week 12 itch and lesion responder analyses also met nominal statistical significance. Non-multiplicity adjusted secondary endpoints including the proportion of responders who reached ≥4-point reduction of WI-NRS at 12 weeks, the proportion of responders who reached an IGA PN-S score of 0 or 1 at 12 weeks, and LS mean change in sleep NRS were all p<0.003.

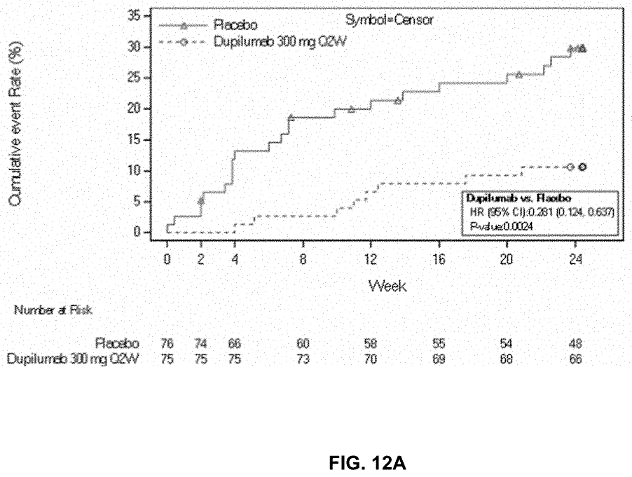

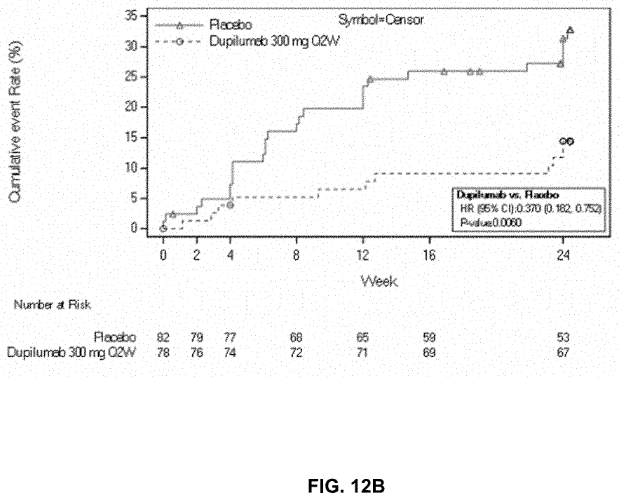

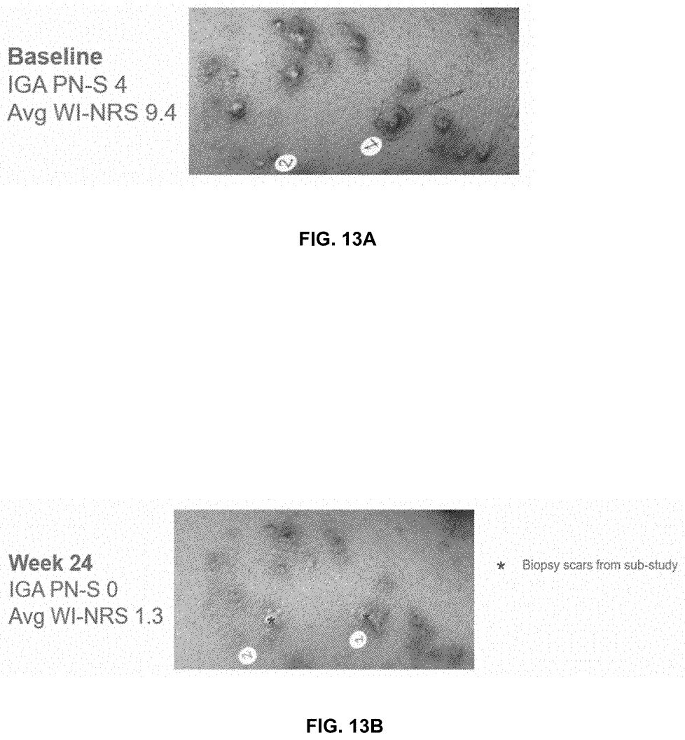

Additionally, as shown in FIG. 12A, dupilumab treatment as compared to placebo reduced the time to first use of rescue and/or prohibited medications or procedures. Overall, dupilumab treatment reduced the use of rescue/prohibited treatment throughout the 24-week intervention period.

Notably, treatment with dupilumab showed differentiation from placebo as early as week 4 in itch responder analyses, did not plateau by week 24, and was consistent regardless of atopic status. It was also noted that symptoms started to relapse after treatment discontinuation.

Dupilumab demonstrated an acceptable safety profile in PN patients with no new safety signal. The safety profile was consistent with the known safety profile of dupilumab observed in the approved populations and indications.

### Results for PRIME2/EFC16460

As shown in FIG. 6, PRIME2/EFC16460 is one of two phase 3 studies that included 78 participants for treatment with dupilumab and 82 participants for treatment with placebo and took place in the United States. 46% of patients enrolled in the trial had at least one coexisting type 2 inflammatory condition. The baseline disease characteristics for patients in the PRIME2/EFC16460 study are shown in FIG. 14. The total enrolled patients had a mean (SD) WI-NRS of 8.5 (1.0) at baseline. Almost all patients in the trial had severe itch with all patients having moderate-to-severe disease based on number of lesions. 62% of enrolled patients had ≥20 to 100 nodules and 38% had >100 nodules.

The primary endpoint was met with clinical and statistical significance. As shown in FIG. 8B, the proportion of participants who reached ≥4-point reduction of Worst-Itch Numeric Rating Scale, WI-NRS (0-10) at week 12 with placebo was 18 (22.0%), and with dupilumab was 29 (37.2%), p=0.0216.

All key secondary endpoints were met with clinical and statistical significance. As shown in FIG. 8B, the proportion of participants who reached ≥4-point reduction of WI-NRS (0-10) at week 24 with placebo was 19.5%, and with dupilumab was 57.7%, (p<0.0001). Thus, at week 24, nearly three times as many dupilumab treated participants experienced a clinically meaningful reduction in itch from baseline. As shown in FIG. 9B, the proportion of participants who reached an Investigator's Global Assessment PN-Stage (IGA PN-S) score of 0 or 1 (0-4) at week 24 with placebo was 15.9%, and with dupilumab was 44.9%, (p<0.0001). Thus, nearly three times as many dupilumab treated participants achieved clear or almost clear skin at week 24. As shown in FIG. 10B, the proportion of participants with concomitant improvement (reduction) in WI-NRS by ≥4 from baseline to week 24 and an IGA PN-S 0 or 1 score at week 24 with placebo was 8.5%, and with dupilumab was 32.1%, (p=0.0001).

As shown in FIG. 7B, several multiplicity adjusted secondary and other efficacy endpoints were also met including the proportion of responders who reached an IGA PN-S score of 0 or 1 at week 12, WI-NRS % mean A from baseline at week 24 (FIG. 11B), DLQI, and skin pain-NRS. The hierarchy broke at second to last (sleep-NRS not significant; HADS nominal).

Additionally, as shown in FIG. 12B, dupilumab treatment as compared to placebo reduced the time to first use of rescue and/or prohibited medications or procedures.

Mild active atopic dermatitis represented 9% of the atopic PN study population and 4.5% of the total study population.

Dupilumab was well-tolerated and demonstrated an acceptable safety profile in PN patients. The safety profile was consistent with the known safety profile of dupilumab observed in the approved populations and indications. No new safety signals and no malignancies were reported with dupilumab.

The pharmacokinetics (PK) and ADA were consistent with the known profile of dupilumab.

### Conclusions for PRIME/EFC16459 and PRIME2/EFC16460

Overall, dupilumab demonstrated clinically and statistically significant efficacy in patients with prurigo nodularis (PN) who were inadequately controlled on topical prescription therapies or for whom those therapies are not advisable. Dupilumab also demonstrated replication of efficacy between the PRIME and PRIME2 studies. Dupilumab was effective and safe for this population, demonstrating an acceptable safety profile.

Approximately three times as many patients on dupilumab experienced significantly reduced itch and skin lesions compared to placebo at 24 weeks. Dupilumab is the first and only biologic to demonstrate positive Phase 3 results in prurigo nodularis.

Dupilumab significantly improved itch and skin clearance at 12 weeks and nearly tripled both at 24 weeks. A significant reduction in itch and skin lesions was observed, which was important given that prior to enrollment nearly all patients had severe itch, and nearly 40% had 100 or more nodules covering their body.

A significant and continuous treatment effect with dupilumab was observed at week 24 across all disease components including itch and lesion severity, regardless of baseline atopic status. In addition, dupilumab improved quality of life and mental health. Dupilumab-treated patients experienced significantly greater improvements in measures of health-related quality of life and skin pain at 24 weeks. Dupilumab-treated patients experienced significantly greater improvements in measures of health-related quality of life, skin pain and symptoms of anxiety and depression.

Dupilumab treatment reduced the use of rescue/prohibited treatment throughout the 24-week intervention period.

About three times as many dupilumab patients (60% and 58%) experienced a clinically meaningful reduction in itch from baseline at 24 weeks, compared to 18% and 20% for placebo, the primary endpoint in PRIME.

44% and 37% of dupilumab patients experienced a clinically meaningful reduction in itch from baseline at 12 weeks, compared to 16% and 22% for placebo, the primary endpoint in PRIME2.

More than twice as many dupilumab patients (48% and 45%) achieved clear or almost clear skin at 24 weeks, compared to 18% and 16% for placebo.

More than three times as many dupilumab patients (39% and 32%) experienced both a clinically meaningful reduction in itch and clear or almost clear skin, compared to 9% and 9% of placebo patients at 24 weeks.

The U.S. Food and Drug Administration has approved dupilumab for the treatment of PN: “DUPIXENT is indicated for the treatment of adult patients with prurigo nodularis (PN). The recommended dosage for adult patients is an initial dose of 600 mg (two 300 mg injections), followed by 300 mg given every other week (Q2W).”

### Example 2: Validation of the Worst-Itch Numeric Rating Scale (WI-NRS) in Prurigo Nodularis (PN) Based on Clinical Studies of Dupilumab in Adults with PN

### Materials and Methods:

Content validity of WI-NRS was assessed through qualitative interviews with adult PN patients (N=20; age: 19-72 years). Measurement properties and within-patient clinical meaningful change (responder definition) of WI-NRS in patients with PN were evaluated using the pooled, blinded data from the phase-3 trials (N=311).

### Results:

Patients found the question, recall period and response scale easy to understand and relevant. Adequate test-retest reliability was observed between screening and baseline (intraclass correlation coefficient=0.72, using Patient Global Impression of Severity (PGIS) to define stable patients). Convergent and divergent validity was supported by moderate-to-strong correlations (r=0.34-0.73) with other conceptually-related measures and weaker correlations (r=0.06-0.32) with less-related measures, respectively. WI-NRS was sensitive to change, as demonstrated by differences in change from baseline among groups (per PGIS change and PGI-change (PGIC); P<0.001). Using anchor-based approach with PGIS and PGIC, the responder definition threshold for improvement was 4-points (range: 3.0-4.5).

### CONCLUSIONS

WI-NRS is a fit-for-purpose instrument to support efficacy endpoints measuring the intensity of pruritus in adults with PN uncontrolled on topical therapies.

1. A method for treating a subject having prurigo nodularis comprising administering to the subject:
an initial dose of about 600 mg of an antibody or an antigen-binding fragment thereof that specifically binds to interleukin-4 receptor (IL-4R) comprising three heavy chain complementarity determining region (HCDR) sequences comprising SEQ ID NOs: 3, 4, and 5, and three light chain complementarity determining region (LCDR) sequences comprising SEQ ID NOs: 6, 7, and 8, and
one or more secondary doses of about 300 mg of the antibody or the antigen-binding fragment thereof.

2. The method of claim 1, wherein the secondary doses are administered every other week (q2w).

3. A method for the treatment of prurigo nodularis that reduces or eliminates a prurigo nodularis patient's dependence on low to medium potency topical corticosteroids and/or topical calcineurin inhibitors comprising:
(a) selecting a patient with prurigo nodularis that is uncontrolled with a background therapy comprising low to medium potency topical corticosteroids and/or topical calcineurin inhibitors;
(b) administering to the patient a defined dose of an antibody or antigen-binding fragment thereof that specifically binds to an interleukin-4 receptor (IL-4R) at a defined frequency for an initial treatment period while maintaining the patient's background therapy for the initial treatment period; and
(c) gradually reducing or eliminating the dosage of low to medium potency topical corticosteroids and/or topical calcineurin inhibitors administered to the patient over the course of a subsequent treatment period while continuing to administer the antibody or antigen-binding fragment thereof at the defined frequency and dose used during the initial treatment period,
wherein the antibody or antigen-binding fragment thereof comprises three heavy chain complementarity determining region (HCDR) sequences comprising SEQ ID NOs: 3, 4, and 5, and three light chain complementarity determining region (LCDR) sequences comprising SEQ ID NOs: 6, 7, and 8.

4. The method of claim 3, wherein:
the antibody or antigen-binding fragment thereof is administered to the subject as an initial dose followed by one or more secondary doses;
the initial dose is about 300 mg and each secondary dose is about 300 mg;
the initial dose is about 600 mg and each secondary dose is about 300 mg; or
the secondary doses are administered every other week (q2w).

5-7. (canceled)

8. A method for treating a subject having prurigo nodularis, or a method for treating pruritis associated with prurigo nodularis, the method comprising administering to the subject:
an initial dose of an antibody or an antigen-binding fragment thereof that specifically binds to interleukin-4 receptor (IL-4R) comprising three heavy chain complementarity determining region (HCDR) sequences comprising SEQ ID NOs: 3, 4, and 5, and three light chain complementarity determining region (LCDR) sequences comprising SEQ ID NOs: 6, 7, and 8, and
one or more secondary doses of the antibody or the antigen-binding fragment thereof,
wherein the treatment with the antibody or antigen-binding fragment thereof results in a decrease in the need for treatment of the subject with superpotent topical corticosteroid rescue medication, or
wherein the treatment with the antibody or antigen-binding fragment thereof results in a decrease in the need for treatment of the subject with systemic immunosuppressants.

9. The method of claim 8, wherein:
the initial dose is about 300 mg and each secondary dose is about 300 mg;
the initial dose is about 600 mg and each secondary dose is about 300 mg; or
the secondary doses are administered every other week (q2w).

10-15. (canceled)

16. The method of claim 8, wherein the pruritus is refractory to topical therapy.

17-20. (canceled)

21. A method for treating a subject having prurigo nodularis comprising administering to the subject:
an initial dose of an antibody or an antigen-binding fragment thereof that specifically binds to interleukin-4 receptor (IL-4R) comprising three heavy chain complementarity determining region (HCDR) sequences comprising SEQ ID NOs: 3, 4, and 5, and three light chain complementarity determining region (LCDR) sequences comprising SEQ ID NOs: 6, 7, and 8, and
one or more secondary doses of the antibody or the antigen-binding fragment thereof, and
wherein the treatment results in the subject having a decrease in worst itch numeric rating scale (WI-NRS) score, or
wherein the treatment results in the subject having a decrease in investigator's global assessment for prurigo nodularis (IGA PN) score, or
wherein the subject has co-morbid mild atopic dermatitis.

22. The method of claim 21, wherein the decrease in WI-NRS score is selected from the group consisting of 4, 5, 6, 7, 8, 9, and 10.

23. The method of claim 21 or 22, wherein the decrease in WI-NRS score occurs with 12 weeks or with 24 weeks of treatment.

24. (canceled)

25. The method of claim 21, wherein the initial dose is about 300 mg and each secondary dose is about 300 mg, or wherein the initial dose is about 600 mg and each secondary dose is about 300 mg.

26. (canceled)

27. The method of claim 21, wherein the secondary doses are administered every other week (q2w).

28. (canceled)

29. The method of claim 21, wherein the decrease in IGA PN score is selected from the group consisting of 5, 4, 3, 2, and 1.

30. The method of claim 21, wherein the subject achieves an IGA PN score of 0 or 1.

31. The method of claim 21, wherein the decrease in IGA PN score occurs with 12 weeks or with 24 weeks of treatment.

32-39. (canceled)

40. The method of claim 1, wherein the subject:
was previously ineffectively treated with medium-to-superpotent topical corticosteroids;
has a baseline WI-NRS score that is equal to or greater than 7;
has a minimum of 20 PN nodules in total on both legs, and/or both arms and/or trunk at baseline;
has a baseline IGA PN score of greater than or equal to 3;
has PN that is not adequately controlled with topical therapies or when those therapies are not advisable; or
is a candidate for systemic therapy.

41-45. (canceled)

46. The method of claim 1, wherein the antibody or antigen-binding fragment thereof comprises a heavy chain variable region (HCVR) sequence of SEQ ID NO: 1 and a light chain variable region (LCVR) sequence of SEQ ID NO: 2, optionally wherein the antibody is dupilumab.

47. (canceled)

48. The method of claim 1, wherein the antibody or antigen-binding fragment thereof is administered using an autoinjector, a needle and syringe, or a pen, optionally wherein the antibody or antigen-binding fragment thereof is administered using a prefilled device, and/or wherein the antibody or antigen-binding fragment thereof is administered subcutaneously.

49. (canceled)

50. (canceled)

51. The method of claim 1, wherein the subject is an adult.

52. A method for treating a subject having prurigo nodularis comprising:
selecting a subject having prurigo nodularis; and
administering to the subject an initial dose of about 600 mg of an antibody or an antigen-binding fragment thereof that specifically binds to interleukin-4 receptor (IL-4R) comprising three heavy chain complementarity determining region (HCDR) sequences comprising SEQ ID NOs: 3, 4, and 5, and three light chain complementarity determining region (LCDR) sequences comprising SEQ ID NOs: 6, 7, and 8, and one or more secondary doses of about 300 mg of the antibody or the antigen-binding fragment thereof.

53. (canceled)

54. (canceled)


SEQUENCE LISTING (295 sequences)

SEQ ID NO: 1 - protein, 124 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, synthetic construct [sequence omitted]

SEQ ID NO: 3 - protein, 8 aa, synthetic construct
GFTFRDYA

SEQ ID NO: 4 - protein, 8 aa, synthetic construct
ISGSGGNT

SEQ ID NO: 5 - protein, 16 aa, synthetic construct
AKDRLSITIRPRYYGL

SEQ ID NO: 6 - protein, 11 aa, synthetic construct
QSLLYSIGYNY

SEQ ID NO: 7 - skipped

SEQ ID NO: 8 - protein, 9 aa, synthetic construct
MQALQTPYT

SEQ ID NO: 9 - protein, 451 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 219 aa, synthetic construct
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYGSSPPWTFGQGTKVEIK

SEQ ID NO: 12 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPWTFGQGTKVEIK

SEQ ID NO: 13 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYGSSPPWTFGQGTKVEIK

SEQ ID NO: 14 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPPWTFGQGTKVEIK

SEQ ID NO: 15 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYGSSPPWTFGQGTKVEIK

SEQ ID NO: 16 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYGSSPPWTFGQGTKVEIK

SEQ ID NO: 17 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSPPWTFGQGTKVEIK

SEQ ID NO: 18 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSAGWTFGQGTKVEIK

SEQ ID NO: 19 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSAGWTFGQGTKVEIK

SEQ ID NO: 20 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSPPWTFGQGTKVEIK

SEQ ID NO: 21 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSPPWTFGQGTKVEIK

SEQ ID NO: 22 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSPPWTFGQGTKVEIK

SEQ ID NO: 23 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYDHSAGWTFGQGTKVEIK

SEQ ID NO: 24 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYDHSAGWTFGQGTKVEIK

SEQ ID NO: 25 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYDHSAGWTFGQGTKVEIK

SEQ ID NO: 26 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSAGWTFGQGTKVEIK

SEQ ID NO: 27 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSAGWTFGQGTKVEIK

SEQ ID NO: 28 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSAGWTFGQGTKVEIK

SEQ ID NO: 29 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSPPWTFGQGTKVEIK

SEQ ID NO: 30 - protein, 109 aa, synthetic construct
EIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIK

SEQ ID NO: 31 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 32 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 33 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 34 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFDYWGQGTLVTVSS

SEQ ID NO: 35 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFDYWGQGTLVTVSS

SEQ ID NO: 36 - protein, 115 aa, synthetic construct
EVQLVESGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 37 - protein, 115 aa, synthetic construct
EVQLVESGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 38 - protein, 115 aa, synthetic construct
EVQLVQSGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 39 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 40 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 41 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 42 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 43 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 44 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 45 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 46 - protein, 115 aa, synthetic construct
EVQLVQSGGGLVHPGRSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 47 - protein, 115 aa, synthetic construct
EVQLVQSGGGLVHPGGSLRLTCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 48 - protein, 115 aa, synthetic construct
EVQLVQSGGGLVHPGGSLRLSCAGSGFTFSRNAMHWVRQAPGKGLEWVSGIGTGGATNYADSVKGRFTISRDNAKNSLYLQMNSLRAEDMAVYYCARGRYYFDYWGQGTLVTVSS

SEQ ID NO: 49 - protein, 115 aa, synthetic construct
EVQLVQSGGGLVHPGGSLRLSCAGSGFTFSRNAMFWVRQAPGEGLEWVSGIGTGGATNYADSVKGRFTISRDNAKNSLYLQMNSLRAEDMAVYYCARGRYYFDYWGQGTLVTVSS

SEQ ID NO: 50 - protein, 115 aa, synthetic construct
EVQLVQSGGGLVHPGGSLRLSCAGSGFTFSRNAMFWVRQAPGKGLEWVSGIGTGGATNYADSVKGRFTISRDEAKNSLYLQMNSLRAEDMAVYYCARGRYYFDYWGQGTLVTVSS

SEQ ID NO: 51 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAGDMAVYYCARGRYYFDYWGQGTLVTVSS

SEQ ID NO: 52 - protein, 115 aa, synthetic construct
EVQLVQSGGGLVHPGGSLRLSCAGSGFTFDDYAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 53 - protein, 115 aa, synthetic construct
EVQLVQSGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 54 - protein, 115 aa, synthetic construct
EVQLVESGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 55 - protein, 115 aa, synthetic construct
EVQLVESGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 56 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 57 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 58 - protein, 115 aa, synthetic construct
EVQLVQSGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 59 - protein, 115 aa, synthetic construct
EVQLVQSGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 60 - protein, 115 aa, synthetic construct
EVQLVESGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 61 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 62 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 63 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 64 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 65 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 66 - protein, 5 aa, synthetic construct
RNAMF

SEQ ID NO: 67 - protein, 16 aa, synthetic construct
GIGTGGATSYADSVKG

SEQ ID NO: 68 - protein, 7 aa, synthetic construct
GRYYFDY

SEQ ID NO: 69 - protein, 12 aa, synthetic construct
RASQSVSSSYLA

SEQ ID NO: 70 - protein, 7 aa, synthetic construct
GASSRAT

SEQ ID NO: 71 - protein, 10 aa, synthetic construct
QQYDHSAGWT

SEQ ID NO: 72 - protein, 7 aa, synthetic construct
GASSRAP

SEQ ID NO: 73 - protein, 10 aa, synthetic construct
QQYGSSPPWT

SEQ ID NO: 74 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLDYWGKGTLVTVSS

SEQ ID NO: 75 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSLSANYVFGTGTKLTVL

SEQ ID NO: 76 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYNWGKGTLVTVSS

SEQ ID NO: 77 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSQPPNPLFGTGTKLTVL

SEQ ID NO: 78 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKLLKNPWGKGTLVTVSS

SEQ ID NO: 79 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWFGTPASNYVFGTGTKLTVL

SEQ ID NO: 80 - protein, 116 aa, synthetic construct; identical to SEQ ID NO: 76

SEQ ID NO: 81 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSSPPQPIFGTGTKLTVL

SEQ ID NO: 82 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYDWGKGTLVTVSS

SEQ ID NO: 83 - protein, 111 aa, synthetic construct; identical to SEQ ID NO: 81

SEQ ID NO: 84 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 85 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTYHPIFGTGTKLTVL

SEQ ID NO: 86 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQYWGKGTLVTVSS

SEQ ID NO: 87 - protein, 111 aa, synthetic construct; identical to SEQ ID NO: 81

SEQ ID NO: 88 - protein, 116 aa, synthetic construct; identical to SEQ ID NO: 86

SEQ ID NO: 89 - protein, 111 aa, synthetic construct; identical to SEQ ID NO: 85

SEQ ID NO: 90 - protein, 116 aa, synthetic construct; identical to SEQ ID NO: 76

SEQ ID NO: 91 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTMYPLFGTGTKLTVL

SEQ ID NO: 92 - protein, 116 aa, synthetic construct; identical to SEQ ID NO: 82

SEQ ID NO: 93 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVLTPIFGTGTKLTVL

SEQ ID NO: 94 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWFYDWGKGTLVTVSS

SEQ ID NO: 95 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPSMIPLFGTGTKLTVL

SEQ ID NO: 96 - protein, 116 aa, synthetic construct; identical to SEQ ID NO: 94

SEQ ID NO: 97 - protein, 111 aa, synthetic construct; identical to SEQ ID NO: 91

SEQ ID NO: 98 - protein, 116 aa, synthetic construct; identical to SEQ ID NO: 82

SEQ ID NO: 99 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTLQPLFGTGTKLTVL

SEQ ID NO: 100 - protein, 116 aa, synthetic construct; identical to SEQ ID NO: 76

SEQ ID NO: 101 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPPTKPLFGTGTKLTVL

SEQ ID NO: 102 - protein, 116 aa, synthetic construct; identical to SEQ ID NO: 76

SEQ ID NO: 103 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTHRHPLFGTGTKLTVL

SEQ ID NO: 104 - protein, 116 aa, synthetic construct; identical to SEQ ID NO: 76

SEQ ID NO: 105 - protein, 111 aa, synthetic construct; identical to SEQ ID NO: 85

SEQ ID NO: 106 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQHWGKGTLVTVSS

SEQ ID NO: 107 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVDRPIFGTGTKLTVL

SEQ ID NO: 108 - protein, 116 aa, synthetic construct; identical to SEQ ID NO: 106

SEQ ID NO: 109 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTPMPVFGTGTKLTVL

SEQ ID NO: 110 - protein, 116 aa, synthetic construct; identical to SEQ ID NO: 106

SEQ ID NO: 111 - protein, 111 aa, synthetic construct; identical to SEQ ID NO: 85

SEQ ID NO: 112 - protein, 116 aa, synthetic construct; identical to SEQ ID NO: 84

SEQ ID NO: 113 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVWEWPFGTGTKLTVL

SEQ ID NO: 114 - protein, 116 aa, synthetic construct
QVQLVQSGAEVKKPGASVKVSCKASGYAFTSYYMHWARQAPGQGLEWMGIINPSGGSASYAQKFQGRVTMTRDTSTSTVYMELSSLRSEDTAVYYCARGKYWMYDWGKGTLVTVSS

SEQ ID NO: 115 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEAVYFCGTWDTSTVWEWPFGTGTKLTVL

SEQ ID NO: 116 - protein, 116 aa, synthetic construct; identical to SEQ ID NO: 84

SEQ ID NO: 117 - protein, 111 aa, synthetic construct
QPVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 118 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 119 - protein, 111 aa, synthetic construct
QSVLTQPPSVSAAPGQKVTISCSGGSSNIGNNYVSWYQQLPGTAPKLLIYDNNKRPPGIPDRFSGSKSGTSATLAITGLQTGDEADYYCGTWDTSTVWEWPFGTGTKLTVL

SEQ ID NO: 120 - protein, 116 aa, synthetic construct
QVQLVQSGAEVKKPGASVKVSCKASGYAFTSYYMHWARQAPGQGLEWMGIINPRGGSTSYAQKFQGRVTMTRDTSTSTVYMELSSLRSEDTAVYYCARGKYWMYDWGKGTLVTVSS

SEQ ID NO: 121 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 122 - protein, 116 aa, synthetic construct
QVQLVQSGAEVKKPGASVKVSCKASGYAFTSYYMHWARQAPGQGLEWMGIINPRGGSASYAQKFQGRVSMTRDTSTSTVYMELSSLRSEDTAVYYCARGKYWMYDWGKGTLVTVSS

SEQ ID NO: 123 - protein, 111 aa, synthetic construct
QSVLTQPPSVSAAPGQKVTISCSGGSSNIGNSYVSWYQQLPGTAPKLLIYDNNKRPSGIPDRFSGSKSGTTATLAITGLQTGDEADYYCGTWVTSTVWEWPFGTGTKLTVL

SEQ ID NO: 124 - protein, 116 aa, synthetic construct; identical to SEQ ID NO: 84

SEQ ID NO: 125 - protein, 111 aa, synthetic construct; identical to SEQ ID NO: 121

SEQ ID NO: 126 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTQVTVSS

SEQ ID NO: 127 - protein, 111 aa, synthetic construct
QSVLTQPPLVSAAPGQKVTISCSGGSSNIGNSYVSWYQRLPGTAPKLLIYDNNKRPSGIPDRFSGSKSGTSATLAITGLQTGDEADYYCGTWDTSTVWEWPFGTGTKLTVL

SEQ ID NO: 128 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGNGTLVTVSS

SEQ ID NO: 129 - protein, 111 aa, synthetic construct
LPVLTQPPSVSAAPGQKVTISCSGGSSSIGNSYVSWYQQLPGAAPKLLIYDNNKRPSGIPDRFSGFRSGTSATLAITGLQTGDEADYYCGTWDTSPVWEWPFGTGTKLTVL

SEQ ID NO: 130 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTRVTVSS

SEQ ID NO: 131 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVWEWPFGTGTKLTVL

SEQ ID NO: 132 - protein, 116 aa, synthetic construct; identical to SEQ ID NO: 84

SEQ ID NO: 133 - protein, 111 aa, synthetic construct
QSVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTVWEWPFGTGTKLTVL

SEQ ID NO: 134 - protein, 116 aa, synthetic construct; identical to SEQ ID NO: 84

SEQ ID NO: 135 - protein, 111 aa, synthetic construct
QSVLTQPPSVSAAPGQKVTISCSGGSSSIGNSYVSWYQQLPGTAPKLLIYDNNKRPSGIPDRFSGSKSGTSATLAITGLQTGDEADYYCGTWATSPVWEWPFGTGTKLTVL

SEQ ID NO: 136 - protein, 116 aa, synthetic construct; identical to SEQ ID NO: 84

SEQ ID NO: 137 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTAWEWPFGTGTKLTVL

SEQ ID NO: 138 - protein, 116 aa, synthetic construct
QVQLVQSGAEEKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 139 - protein, 111 aa, synthetic construct
QSALTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 140 - protein, 116 aa, synthetic construct
QVQLVQSGAEVKKPGASVKVSCKASGYAFTSYYMHWARQAPGQGLEWMGIINPSGGSTSYAQKFQGRVSMTRDTSTSTVYMELSSLRSEDTAVYYCARGKYWMYDWGKGTLVTVSS

SEQ ID NO: 141 - protein, 111 aa, synthetic construct; identical to SEQ ID NO: 121

SEQ ID NO: 142 - protein, 116 aa, synthetic construct; identical to SEQ ID NO: 84

SEQ ID NO: 143 - protein, 111 aa, synthetic construct; identical to SEQ ID NO: 131

SEQ ID NO: 144 - protein, 116 aa, synthetic construct; identical to SEQ ID NO: 114

SEQ ID NO: 145 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDSSTVWEWPFGTGTKLTVL

SEQ ID NO: 146 - protein, 116 aa, synthetic construct
QVQLVQSGAEVKKPGASVKVSCKASGYAFTSYYMHWARQAPGQGLEWMGIINPRGGSTSYAQKFQGRVAMTRDTSTSTVYMELSSLRPEDTAVYYCARGKYWMYDWGKGTLVTVSS

SEQ ID NO: 147 - protein, 111 aa, synthetic construct
QSVLTQPPSVSAAPGQKVTISCSGGGSSIGNSYVSWYQQLPGTAPKLLIYDNNKRPSGVPDRFSGSKSGTSATLAITGLQTGDEADYYCGTWDTSPVWEWPFGTGTKLTVL

SEQ ID NO: 148 - protein, 116 aa, synthetic construct; identical to SEQ ID NO: 114

SEQ ID NO: 149 - protein, 111 aa, synthetic construct; identical to SEQ ID NO: 121

SEQ ID NO: 150 - protein, 116 aa, synthetic construct; identical to SEQ ID NO: 120

SEQ ID NO: 151 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTAWEWPFGTGTKLTVL

SEQ ID NO: 152 - protein, 116 aa, synthetic construct; identical to SEQ ID NO: 84

SEQ ID NO: 153 - protein, 111 aa, synthetic construct; identical to SEQ ID NO: 145

SEQ ID NO: 154 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTLVTVSG

SEQ ID NO: 155 - protein, 111 aa, synthetic construct
QSVLTQPPSVSAAPGQKVTISCSGGSTNIGNSYVSWYQRLPGTAPKLLIYDNNKRPPGIPDRFSGSKSGTSATLAITGLQTGDEADYYCGTWDTSTVWEWPFGTGTKLTVL

SEQ ID NO: 156 - protein, 116 aa, synthetic construct
QVQLVQSGAEVKKPGASVKVSCKASGYAFTSYYMHWARQAPGQGLEWVGIINPSGGSTSYAQKFQGRVTMTRDTSTSTVYMELSSLRSGDTAVYYCARGKYWMYDWGKGTLVTVSS

SEQ ID NO: 157 - protein, 111 aa, synthetic construct
QAVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTGWEWPFGTGTKLTVL

SEQ ID NO: 158 - protein, 116 aa, synthetic construct
QVQLVQSGAEVKKPGASVKVSCKASGYAFTSYYMHWVRQAPGQGLEWMGIINPRGGSTSYAQKFQGRVTMTRDTSTSTVYMELSSLRSEDTAVYYCARGKYWMYDWGKGTLVTVSS

SEQ ID NO: 159 - protein, 111 aa, synthetic construct
QSVLTQPPSVSAAPGQKVTISCSGGGSSIGNSYVSWYQQLPGTAPKLLIYDNNKRPSGIPDRFSGSKSGTSATLGITGLQTGDEADYYCGTWDTSPVWEWPFGTGTKLTVL

SEQ ID NO: 160 - protein, 5 aa, synthetic construct
SYYMH

SEQ ID NO: 161 - protein, 17 aa, synthetic construct
IINPRGGSTSYAQKFQG

SEQ ID NO: 162 - protein, 7 aa, synthetic construct
GKYWMYD

SEQ ID NO: 163 - protein, 13 aa, synthetic construct
SGGGSSIGNSYVS

SEQ ID NO: 164 - protein, 7 aa, synthetic construct
DNNKRPS

SEQ ID NO: 165 - protein, 12 aa, synthetic construct
GTWDTSPVWEWP

SEQ ID NO: 166 - protein, 116 aa, synthetic construct
EVQLLESGGGLVQPGGSLRLSCAVSGFTFSNYAMSWVRQAPGKGLEWVSAISSGGGNIYYADSVKGRFTISRDNSKNTLYLQMNSLRAEDTAVYYCAKLRRYFDYWGQGTLVTVSS

SEQ ID NO: 167 - protein, 121 aa, synthetic construct
EVQLLESGGGLVQPGGSLRLSCAASGFTFSDYAMSWVRQAPGKGLEWVSAISSGGSSIYYADSVKGRFTISRDNSKNTLHLQMNSLRAEDTAVYYCARGPQRSATAVFDYWGQGTLVTVSS

SEQ ID NO: 168 - protein, 127 aa, synthetic construct
EVQLLESGGGLVQPGGSLRLSCAASGFTFSNYAMSWVRQAPGKGLEWVSWISPNSGNIYYADSVKGRFTISRDNSKNTLYLQMNSLRAEDTAVYYCARRPLSAAWSHSSYYNAMDVWGQGTLVTVSS

SEQ ID NO: 169 - protein, 116 aa, synthetic construct
EVQLLESGGGLVQPGGSLRLSCAASGFTFSGYAMSWVRQAPGKGLEWVSLISHSGSNTYYADSVKGRFTISRDNSKNTLYLQMNSLRAEDTAVYYCARPHRAFDYWGQGTLVTVSS

SEQ ID NO: 170 - protein, 116 aa, synthetic construct
EVQLLESGGGLVQPGGSLRLSCAASGFTFSNYAMSWVRQAPGKGLEWVSGISHGSGSIYYADSVKGRFTISRDNSKNTLYLQMNSLRAEDTAVYYCARPHRAFDYWGQGTLVTVSS

SEQ ID NO: 171 - protein, 116 aa, synthetic construct
EVQLLESGGGLVQPGGSLRLSCAASGFTFSNYAMSWVRQAPGKGLEWVSGISHGNGSIYYADSVKGRFTISRDNSKNTLYLQMNSLRAEDTAVYYCAKTGRHFDYWGQGTLVTVSS

SEQ ID NO: 172 - protein, 116 aa, synthetic construct
EVQLLESGGGLVQPGGSLRLSCAASGFTFSNYAMSWVRQAPGKGLEWVSSISPSGSSIYYADSVKGRFTISRDNSKNTLYLQMNSLRAEDTAVYYCARSYRAFDYWGQGTLVTVSS

SEQ ID NO: 173 - protein, 116 aa, synthetic construct
EVQLLESGGGLVQPGGSLRLSCAASGFTFSNYAMSWVRQAPGKGLEWVSAISPSGGSIYYADSVKGRFTISRDNSKNTLYLQMNSLRAEDTAVYYCARAKRAFDYWGQGTLVTVSS

SEQ ID NO: 174 - protein, 116 aa, synthetic construct
EVQLLESGGGLVQPGGSLRLSCAASGFTFSNYAMSWVRQAPGKGLEWVSAISPGSGSTYYADSVKGRFTISRDNSKNTLYLQMNSLRAEDTAVYYCAKFRRHFDYWGQGTLVTVSS

SEQ ID NO: 175 - protein, 116 aa, synthetic construct
EVQLLESGGGLVQPGGSLRLSCAASGFTFSNYAMSWVRQAPGKGLEWVSAISSGGGNIYYADSVKGRFTISRDNSKNTLYLQMNSLRAEDTAVYYCARVHRAFDYWGQGTLVTVSS

SEQ ID NO: 176 - protein, 116 aa, synthetic construct
EVQLLESGGGLVQPGGSLRLSCAASGFTFSNYAMSWVRQAPGKGLEWVSAITSSGRSIYYADSVKGRFTISRDNSKNTLYLQMNSLRAEDTAVYYCARVHRAFDYWGQGTLVTVSS

SEQ ID NO: 177 - protein, 116 aa, synthetic construct
EVQLLESGGGLVQPGGSLRLSCAASGFTFSNYAMSWVRQAPGKGLEWVSAITSSGANIYYADSVKGRFTISRDNSKNTLYLQMNSLRAEDTAVYYCARVHRAFDYWGQGTLVTVSS

SEQ ID NO: 178 - protein, 116 aa, synthetic construct
EVQLLESGGGLVQPGGSLRLSCAASGFTFSNYAMSWVRQAPGKGLEWVSAITSSGGNIYYADSVKGRFTISRDNSKNTLYLQMNSLRAEDTAVYYCARVHRAFDYWGQGTLVTVSS

SEQ ID NO: 179 - protein, 116 aa, synthetic construct
EVQLLESGGGLVQPGGSLRLSCAASGFTFSNYAMSWVRQAPGKGLEWVSAITAGGGSIYYADSVKGRFTISRDNSKNTLYLQMNSLRAEDTAVYYCARVHRAFDYWGQGTLVTVSS

SEQ ID NO: 180 - protein, 116 aa, synthetic construct
EVQLLESGGGLVQPGGSLRLSCAASGFTFSRHAMAWVRQAPGKGLEWVSAITSSGRSIYYADSVKGRFTISRDNSKNTLYLQMNSLRAEDTAVYYCARVHRAFDYWGQGTLVTVSS

SEQ ID NO: 181 - protein, 110 aa, synthetic construct
QSVLTQPPSASGTPGQRVTISCSGSSSNIGNNYVNWYQQLPGTAPKLLIYDNSHRPSGVPDRFSGSKSGTSASLAISGLRSEDEADYYCGTWDASLSAYVFGGGTKLTVL

SEQ ID NO: 182 - protein, 110 aa, synthetic construct
QSVLTQPPSASGTPGQRVTISCSGSSSNIGNNNVSWYQQLPGTAPKLLIYANSKRPSGVPDRFSGSKSGTSASLAISGLRSEDEADYYCGSWDDSLSAYVFGGGTKLTVL

SEQ ID NO: 183 - protein, 110 aa, synthetic construct
QSVLTQPPSAPGTPGQRVTISCTGSSSNIGSNSVNWYQQLPGTAPKLLIYDDSHRPSGVPDRFSGSKSGTSASLAISGLRSEDEADYYCDAWDSSLSAYVFGGGTKLTVL

SEQ ID NO: 184 - protein, 110 aa, synthetic construct
QSVLTQPPSASGTPGQRVTLSCTGSSSNIGSNYVSWYQQLPGTAPKLLIYADSQRPSGVPDRFSGSKSGTSASLAISGLRSEDEADYYCGTWDDSLSGYVFGGGTKLTVL

SEQ ID NO: 185 - protein, 110 aa, synthetic construct
QSVLTQPPSASGTPGQRVTISCSSSSSNIGSNYVSWYQQLPGTAPKLLIYSDSHRPSGVPDRFSGSKSGTSASLAISGLRSEDEADYYCGSWDYSLSAYVFGGGTKLTVL

SEQ ID NO: 186 - protein, 110 aa, synthetic construct
QSVLTQPPSASGTPGQRVTISCTGSSSNIGNNTVSWYQQLPGTAPKLLIYDNSHRPSGVPDRFSGSKSGTSASLAISGLQSEDEADYYCGSWDYSLSAYVFGGGTKLTVL

SEQ ID NO: 187 - protein, 110 aa, synthetic construct
QSVLTQPPSASGTPGQRVTISCTGSSSNIGNNDVNWYQQLPGTAPKLLIYYDSQRPSGVPDRFSGSKSGTSASLAISGLRSEDEADYYCATWDASLSAYVFGGGTKLTVL

SEQ ID NO: 188 - protein, 110 aa, synthetic construct
QSVLTQPPSASGTPGQRVTISCSGSSSNIGSNAVNWYQQLPGTAPKLLIYYDNQRPSGVPDRFSGSKSGTSASLAISGLRSEDEADYYCGTWDDSLNGYVFGGGTKLTVL

SEQ ID NO: 189 - protein, 110 aa, synthetic construct
QSVLTQPPSASGTPGQRVTISCSGSSSNIGNNAVTWYQQLPGTAPKLLIYDDSHRPSGVPDRFSGSKSGTSASLAISGLRSEDEADYYCGSWDYSLSAYVFGGGTKLTVL

SEQ ID NO: 190 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLSGYVLGGGTKLTVL

SEQ ID NO: 191 - protein, 110 aa, synthetic construct; identical to SEQ ID NO: 190

SEQ ID NO: 192 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLRGYVLGGGTKLTVL

SEQ ID NO: 193 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGYWDYSLSGYVLGGGTKLTVL

SEQ ID NO: 194 - protein, 110 aa, synthetic construct; identical to SEQ ID NO: 190

SEQ ID NO: 195 - protein, 111 aa, synthetic construct
QSVLTQPPSASGTPGQRVTISCSGSSANSRTDGFNWYQQLPGTAPKLLIYADSHRPSGVPDRFSGSKSGTSASLAISGLRSEDEADYYCGTWDYSLSGYVLGGGTKLTVLG

SEQ ID NO: 196 - protein, 111 aa, synthetic construct
QSVLTQPPSASGTPGQRVTISCSGSAQFGSRDNFNWYQQLPGTAPKLLIYADSHRPSGVPDRFSGSKSGTSASLAISGLRSEDEADYYCGTWDYSLSGYVLGGGTKLTVLG

SEQ ID NO: 197 - protein, 111 aa, synthetic construct
QSVLTQPPSASGTPGQRVTISCSGSTKQMHNYQFNWYQQLPGTAPKLLIYADSHRPSGVPDRFSGSKSGTSASLAISGLRSEDEADYYCGTWDYSLSGYVLGGGTKLTVLG

SEQ ID NO: 198 - protein, 111 aa, synthetic construct
QSVLTQPPSASGTPGQRVTISCSGSLLRGENLQFNWYQQLPGTAPKLLIYADSHRPSGVPDRFSGSKSGTSASLAISGLRSEDEADYYCGTWDYSLSGYVLGGGTKLTVLG

SEQ ID NO: 199 - protein, 111 aa, synthetic construct
QSVLTQPPSASGTPGQRVTISCSGSPLFPDSGSFNWYQQLPGTAPKLLIYADSHRPSGVPDRFSGSKSGTSASLAISGLRSEDEADYYCGTWDYSLSGYVLGGGTKLTVLG

SEQ ID NO: 200 - protein, 111 aa, synthetic construct
QSVLTQPPSASGTPGQRVTISCSGSAALDLSPSFNWYQQLPGTAPKLLIYADSHRPSGVPDRFSGSKSGTSASLAISGLRSEDEADYYCGTWDYSLSGYVLGGGTKLTVLG

SEQ ID NO: 201 - protein, 5 aa, synthetic construct
RHAMA

SEQ ID NO: 202 - protein, 17 aa, synthetic construct
AITSSGRSIYYADSVKG

SEQ ID NO: 203 - protein, 7 aa, synthetic construct
VHRAFDY

SEQ ID NO: 204 - protein, 13 aa, synthetic construct
SGSPLFPDSGSFN

SEQ ID NO: 205 - protein, 7 aa, synthetic construct
ADSHRPS

SEQ ID NO: 206 - protein, 11 aa, synthetic construct
GTWDYSLSGYV

SEQ ID NO: 207 - protein, 117 aa, synthetic construct
QVQLVQSGAEVKKPGASVKVSCKASGYTFTNYGISWVRQAPGQGLEWMGWISVYNGKTNYAQKLQGRVTMTTDTSTTTAYMEMRSLRSDDTAVYYCARGSGYDLDYWGQGTLVSVSS

SEQ ID NO: 208 - protein, 128 aa, synthetic construct
EVQLVESGGGLVQPGGSLRLSCAASGFTFSSFWMTWVRQAPGKGLEWVANIKQDGSEKYYVDSVKGRFTISRDNAKNSLYLQMNSLRAEDTAVYYCARDPGRTMVRGGIRYYYGMDVWGQGTTVTVSS

SEQ ID NO: 209 - protein, 122 aa, synthetic construct
EVKLAESGGGLVQPGGSLRLSCAASGFTFSSHWMNWVRQAPGKGLEWVANIKQDGSDKYYVDSVKGRFTISRDNAKNSLYLQLNSLIAEDTAVYYCARDRGVRPPRGAFDIWGQGTMVTVSS

SEQ ID NO: 210 - protein, 128 aa, synthetic construct
QVQLVQSGAEVKKPGASVKVSCKASGYTFNSYGISWVRQAPGQGLEWMGWIRTYNGNTNYAQKLQGRVTMTTDTSTSTAYMELRSLRSDDTAVYYCARDEARIVVAGTTPYYYGMDVWGQGTTVTVSS

SEQ ID NO: 211 - protein, 117 aa, synthetic construct
QVQLVESGGGLVQPGGSLRLSCAVSGFTISDHYMSWIRQAPGKGLEWISYISSSGSKIYYADSVKGRFTISRDNAKNSLFLQMNSLRAEDTAVYYCARTRQLVGDYWGQGTLVTVSS

SEQ ID NO: 212 - protein, 124 aa, synthetic construct
EVQLVESGGGLVQPGRSLRLSCAASGFTFDNYAMHWVRQAPGKGLEWVSGIRWNSGSIGYADSVKGRFTISRDNAKNSLYLQMNSLRAEDTALYYCAKEGGYSGYRPGPFFDYWGQGTLVTVSS

SEQ ID NO: 213 - protein, 117 aa, synthetic construct
QVQLVQSGAEVKKPGASVKVSCKASGYTFTNYGISWVRQAPGQGLEWMGWISVYNGHTNYAQKLQGRVTMTTDTSTSTAYMELRSLRSDDTAVYYCARGSGYDFDSWGQGTLVTVSS

SEQ ID NO: 214 - protein, 116 aa, synthetic construct
QVQLVQSGAEVKKPGASVKVSCKASRYTFTSYDINWVRQATGQGLEWMGWMNPNSGNTGYAQKFQGRVTMTRNTSTSTAYMELSSLRSEDTAVYYCARVRRFFDYWGQGTLVTVSS

SEQ ID NO: 215 - protein, 117 aa, synthetic construct
QVQLVQSGPEVKKPGASVKVSCKASGYTFTNYGISWVRQAPGQGLEWMGWISVYNGNINYAQKLQGRVTMTTDTSTSTAYMDLRSLRSDDTAVYYCARGSGYDFDYWGQGTLVTVSS

SEQ ID NO: 216 - protein, 122 aa, synthetic construct
QVQLVQSGAEVKKPGASVKVSCKDSAYTFNRYGISWVRQAPGQGLEWMGWISAYTGNTVYAQKLQGRVTMTTDNSTSTAYMELRSLRSDDTAVYYCARDKSIFGVVRGFDYWGQGTLVTVSS

SEQ ID NO: 217 - protein, 107 aa, synthetic construct
AIQMTQSPSSLSASVGDRVTITCRASQGIRNALGWYQQKPGKAPKLLIYAASSLQSGVPSRFSGSGSGTDFTLTFSSLQPEDFATYYCLQDFNYPYTFGQGTKLEIK

SEQ ID NO: 218 - protein, 107 aa, synthetic construct
DIQMTQSPSSVSASVGDRVTISCRASQGVSSWLAWYQQKPGNAPKLLISAASSIQSGVPSRFSGSGSGTDFTLTISSLQPEDFATYYCQQANSFPLTFGGGTKVEIK

SEQ ID NO: 219 - protein, 107 aa, synthetic construct
DIQMTQSPSSVSASVGDRVTITCRASQGISSWLAWYQQKPGKAPKLLIYAASSFQSGVPSRFSGSGSGTDFTLTISSLQPEDFATYFCQQANSFPLTFGGGTTVEIK

SEQ ID NO: 220 - protein, 107 aa, synthetic construct
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLATK

SEQ ID NO: 221 - protein, 107 aa, synthetic construct
DIQLTQSPSFLSASVGDRVTITCWASQGISSYLAWYQQKPGKAPKLLIFAASTLQSGVPSRFSGSGSGTEFTLTISSLQPEDFATYYCQQLNSYPLTFGGGTKVEIR

SEQ ID NO: 222 - protein, 107 aa, synthetic construct
EIVMTQSPATLSVSPGERATLSCRASQSVNYNLAWYQHKPGQAPRLLIYGASTRATGIPARFSGSGSGTEFTLTISSLQSEDFAVYYCQQYNNWPLTFGGGTKVEIK

SEQ ID NO: 223 - protein, 107 aa, synthetic construct
AIQMTQSSSSLSASVGDRVTITCRASQAIRNALGWYQQKPGKAPKVLIYAASSLQSGIPSRFSGSGSGTDFTLTISSLQPEDFATYYCLQDYDYPYTFGQGTKLEIK

SEQ ID NO: 224 - protein, 107 aa, synthetic construct
DIQLTQSPSFLSASVGDRVTITCWASQGIISYLAWYQQKPGKAPKLLIYAASTLHSGVPSRFSGSGSGTEFTLTISSLQPEDFATYYCHQLKSYPITFGQGTRLEIK

SEQ ID NO: 225 - protein, 107 aa, synthetic construct
AIQMTQSPSSLSASVGDRVTITCRASQDIRNALGWYQQKPGKAPKLLIYAASSLQSGVPSRFSGSASGTDFTLTISSLQPEDFAAYYCLQDYNYPYTFGQGTKLEIK

SEQ ID NO: 226 - protein, 107 aa, synthetic construct
EIVMTQSPVTLSLSPGERATLPCRASQSVSSSLAWYQQKAGQSPRLLIYGASTRATGIPARFSGSGSGTEFTLTISNLQSEDFAVYYCQQYNNWPLTFGGGTKVEIK

SEQ ID NO: 227 - protein, 120 aa, synthetic construct
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVKVGYRGGMDVWGQGTTVTVSS

SEQ ID NO: 228 - protein, 120 aa, synthetic construct
EVQLLESGGGLVQPGGSLRLSCAASGFTLSSYAMHWVRQAPGKGLEYVSGISPSGSSTYYANSVKGRFTISRDNPKNTLFLQMSSLRAEDTAVYYCVRSKVRYRGGMDVWGQGTTVTVSS

SEQ ID NO: 229 - protein, 120 aa, synthetic construct
EVQLLESGGGLVQPGGSLRLSCAASGFTLSSYAMHWVRQAPGKGLEYVSGISPSGVSTYYANSVKGRFTISRDNPKNTLFLQMSSLRAEDTAVYYCVRVKVKYRGGMDVWGQGTTVTVSS

SEQ ID NO: 230 - protein, 120 aa, synthetic construct
EVQLLESGGGLVQPGGSLRLSCAASGFTLSSYAMHWVRQAPGKGLEYVSGISPTSGSTYYANSVKGRFTISRDNPKNTLFLQMSSLRAEDTAVYYCVRVKVRYRGGMDVWGQGTTVTVSS

SEQ ID NO: 231 - protein, 120 aa, synthetic construct
EVQLLESGGGLVQPGGSLRLSCAASGFTLSSYAMHWVRQAPGKGLEYVSGISPTGTSTYYANSVKGRFTISRDNPKNTLFLQMSSLRAEDTAVYYCVRVKGAYRGGMDVWGQGTTVTVSS

SEQ ID NO: 232 - protein, 120 aa, synthetic construct
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKVAYRGGMDVWGQGTTVTVSS

SEQ ID NO: 233 - protein, 120 aa, synthetic construct
EVQLLESGGGLVQPGGSLRLSCAASGFTLSSYAMHWVRQAPGKGLEYVSGISPSSTSTYYANSVKGRFTISRDNPKNTLFLQMSSLRAEDTAVYYCVRVKVLYRGGMDVWGQGTTVTVSS

SEQ ID NO: 234 - protein, 120 aa, synthetic construct
EVQLLESGGGLVQPGGSLRLSCAASGFTLSSYAMHWVRQAPGKGLEYVSGISPSSASTYYANSVKGRFTISRDNPKNTLFLQMSSLRAEDTAVYYCVRVKSKYRGGMDVWGQGTTVTVSS

SEQ ID NO: 235 - protein, 120 aa, synthetic construct
EVQLLESGGGLVQPGGSLRLSCAASGFTLSSYAMHWVRQAPGKGLEYVSGISGNSASTYYANSVKGRFTISRDNPKNTLFLQMSSLRAEDTAVYYCVRVKLKYRGGMDVWGQGTTVTVSS

SEQ ID NO: 236 - protein, 120 aa, synthetic construct
EVQLLESGGGLVQPGGSLRLSCAASGFTLSSYAMHWVRQAPGKGLEYVSGISHSGTSTYYANSVKGRFTISRDNPKNTLFLQMSSLRAEDTAVYYCVRVRVLYRGGMDVWGQGTTVTVSS

SEQ ID NO: 237 - protein, 120 aa, synthetic construct; identical to SEQ ID NO: 229

SEQ ID NO: 238 - protein, 120 aa, synthetic construct
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVFVRYRGGMDVWGQGTTVTVSS

SEQ ID NO: 239 - protein, 120 aa, synthetic construct
EVQLLESGGGLVQPGGSLRLSCAASGFTLSSYAMHWVRQAPGKGLEYVSGISPTSASTYYANSVKGRFTISRDNPKNTLFLQMSSLRAEDTAVYYCVRVKGRYRGGMDVWGQGTTVTVSS

SEQ ID NO: 240 - protein, 120 aa, synthetic construct
EVQLLESGGGLVQPGGSLRLSCAASGFTLSSYAMHWVRQAPGKGLEYVSGISPTGGSTYYANSVKGRFTISRDNPKNTLFLQMSSLRAEDTAVYYCVRVKGRYRGGMDVWGQGTTVTVSS

SEQ ID NO: 241 - protein, 120 aa, synthetic construct
EVQLLESGGGLVQPGGSLRLSCAASGFTLSSYAMHWVRQAPGKGLEYVSGISHSGNSTYYANSVKGRFTISRDNPKNTLFLQMSSLRAEDTAVYYCVRVKRRYRGGMDVWGQGTTVTVSS

SEQ ID NO: 242 - protein, 120 aa, synthetic construct
EVQLLESGGGLVQPGGSLRLSCAASGFTLSSYAMHWVRQAPGKGLEYVSGISPSSNSTYYANSVKGRFTISRDNPKNTLFLQMSSLRAEDTAVYYCVRVKVRYRGGMDVWGQGTTVTVSS

SEQ ID NO: 243 - protein, 120 aa, synthetic construct
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKPAYRGGMDVWGQGTTVTVSS

SEQ ID NO: 244 - protein, 120 aa, synthetic construct
EVQLLESGGGLVQPGGSLRLSCAASGFTLSSYAMHWVRQAPGKGLEYVSGISYSSASTYYANSVKGRFTISRDNPKNTLFLQMSSLRAEDTAVYYCVRVKVRYRGGMDVWGQGTTVTVSS

SEQ ID NO: 245 - protein, 108 aa, synthetic construct
ETTLTQSPDTLPLSPGDRASLSCRASQSVSSAYLAWYQQKPGQAPRLLIYGTSRRATGVPGRFSGSGSGTDFTLTISRLEPEDFAVYYCQLYGSSSVTFGQGTKLEIK

SEQ ID NO: 246 - protein, 108 aa, synthetic construct
EIVLTQSPGTLSLSPGERATLSCRASQGISSAYLAWYQQKPGQAPRLLIYGTSRRATGIPDRFSGSGSGTDFTLTISRLEPEDFAVYYCQLYGATSVTFGQGTKLEIK

SEQ ID NO: 247 - protein, 108 aa, synthetic construct; identical to SEQ ID NO: 246

SEQ ID NO: 248 - protein, 108 aa, synthetic construct
EIVLTQSPGTLSLSPGERATLSCRASQSVSSAYLAWYQQKPGQAPRLLIYGTSRRATGIPDRFSGSGSGTDFTLTISRLEPEDFAVYYCQLYGASSVTFGQGTKLEIK

SEQ ID NO: 249 - protein, 108 aa, synthetic construct; identical to SEQ ID NO: 248

SEQ ID NO: 250 - protein, 108 aa, synthetic construct; identical to SEQ ID NO: 246

SEQ ID NO: 251 - protein, 108 aa, synthetic construct; identical to SEQ ID NO: 248

SEQ ID NO: 252 - protein, 108 aa, synthetic construct
EIVLTQSPGTLSLSPGERATLSCRASQSISTAYLAWYQQKPGQAPRLLIYGTSRRATGIPDRFSGSGSGTDFTLTISRLEPEDFAVYYCQLYGASSVTFGQGTKLEIK

SEQ ID NO: 253 - protein, 108 aa, synthetic construct
EIVLTQSPGTLSLSPGERATLSCRASQDISSAYLAWYQQKPGQAPRLLIYGTSRRATGIPDRFSGSGSGTDFTLTISRLEPEDFAVYYCQLYGATSVTFGQGTKLEIK

SEQ ID NO: 254 - protein, 108 aa, synthetic construct
EIVLTQSPGTLSLSPGERATLSCRASQDVSSAYLAWYQQKPGQAPRLLIYGTSRRATGIPDRFSGSGSGTDFTLTISRLEPEDFAVYYCQLYGATSVTFGQGTKLEIK

SEQ ID NO: 255 - protein, 108 aa, synthetic construct
EIVLTQSPGTLSLSPGERATLSCRASQNISTAYLAWYQQKPGQAPRLLIYGTSRRATGIPDRFSGSGSGTDFTLTISRLEPEDFAVYYCQLYGATSVTFGQGTKLEIK

SEQ ID NO: 256 - protein, 108 aa, synthetic construct
EIVLTQSPGTLSLSPGERATLSCRASQDASNAYLAWYQQKPGQAPRLLIYGTSRRATGIPDRFSGSGSGTDFTLTISRLEPEDFAVYYCQLYGSSSVTFGQGTKLEIK

SEQ ID NO: 257 - protein, 108 aa, synthetic construct
EIVLTQSPGTLSLSPGERATLSCRASQGVSSAYLAWYQQKPGQAPRLLIYGTSRRATGIPDRFSGSGSGTDFTLTISRLEPEDFAVYYCQLYGRSSVTFGQGTKLEIK

SEQ ID NO: 258 - protein, 108 aa, synthetic construct
EIVLTQSPGTLSLSPGERATLSCRASQNISTAYLAWYQQKPGQAPRLLIYGTSRRATGIPDRFSGSGSGTDFTLTISRLEPEDFAVYYCQLYGTSSVTFGQGTKLEIK

SEQ ID NO: 259 - protein, 108 aa, synthetic construct
EIVLTQSPGTLSLSPGERATLSCRASQSVSTAYLAWYQQKPGQAPRLLIYGTSRRATGIPDRFSGSGSGTDFTLTISRLEPEDFAVYYCQLYGATSVTFGQGTKLEIK

SEQ ID NO: 260 - protein, 108 aa, synthetic construct; identical to SEQ ID NO: 253

SEQ ID NO: 261 - protein, 108 aa, synthetic construct
EIVLTQSPGTLSLSPGERATLSCRASQGVSTAYLAWYQQKPGQAPRLLIYGTSRRATGIPDRFSGSGSGTDFTLTISRLEPEDFAVYYCQLYGATSVTFGQGTKLEIK

SEQ ID NO: 262 - protein, 108 aa, synthetic construct
EIVLPQSPGTLSLSPGERATLSCRASQGVSSAYLAWYQQKPGQAPRLLIYGTSRRATGIPDRFSGSGSGTDFTLTISRLEPEDFAVYYCQLYGSTSVTFGQGTKLEIK

SEQ ID NO: 263 - protein, 118 aa, synthetic construct
EVQLVESGGGLVQPKGSLKLSCAASGFTFNTYGMHWVRQAPGKGLEWVAHIRSKSSNYATYYADSVKDRFTISRDDSQSMLYLQMNNLKTEDTAMYYCVRWFRAMDYWGQGTSVTVSS

SEQ ID NO: 264 - protein, 120 aa, synthetic construct
EVQLIESGGGLVQPKGSLKLSCAASGFTFNMYAMDWVRQAPGKGLEWVARIRSKGSNFETNYADSVKDRFTISRDDSQSMVYLQMINLKTEDTAMYYCVRHRGGAWFAYWGQGTLVSVSA

SEQ ID NO: 265 - protein, 121 aa, synthetic construct
QVQLVETGGGLVRPGNSLKLSCVTSGFTFSNYRMHWLRQPPGKRLEWIAVITVKSNNYGANYAESVKGRFAISRDDSKSSVYLEMNRLREEDTATYFCSRERAYGNPFDYWGQGTTLTVSS

SEQ ID NO: 266 - protein, 118 aa, synthetic construct
EVQLVESGGGLVQPKGSLKLSCAASGFTFNMYAMNWVRQAPGQGLEWVARIRSKSNNYATYYADSVKDRFIISRDDSESMVYLQMSNLRAADTAMYYCVRHLRAMDYWGQGTSVTVSS

SEQ ID NO: 267 - protein, 118 aa, synthetic construct
EVQLVESGGGLVQPKGSLKLSCAASGFSFNMYAMNWVRQAPGKGLEWVARIRTKSNHYSTYYADSVKDRFTISRDDSASMFYLQMNNLKTEDTAMYFCVRHLRAMDYWGQGTSVTVSS

SEQ ID NO: 268 - protein, 120 aa, synthetic construct
EVQLIESGGGLVQPKGSLKLSCAASGFTFNMYAMDWVRQAPGKGLEWVARIRSKGSNFETNYADSVKDRFTISRDDSQSMVYLQMNNLKTEDTAMYYCVRHRGGAWFAYWGQGTLVTVSA

SEQ ID NO: 269 - protein, 118 aa, synthetic construct
EVQLVESGGGLVRPKGSLKLSCAASGFSFNTYAMNWVRQAPGKGLEWIVWIRSKSHNYATYYADSVKDRFTISRDDSESMLYLQMNNLKTEDTAMYYCVRHLRAMDYWGQGTSVTVSS

SEQ ID NO: 270 - protein, 118 aa, synthetic construct
EVRLVESGGGLVQPKGSLKLSCEASGFSFNMYAMNWVRQAPGKGLEWITHIRSKSNNYATYYADSVKDRFIISRDDSESMVYLQMNNLKTEDTAMYYCVRLLRALDYWGQGTSVTVSS

SEQ ID NO: 271 - protein, 118 aa, synthetic construct
EVQLVESGGGLVQPKGSLKLSCAASGFTFNMYGMHWVRQAPGKGLEWVAHIRSKSSNYATYYADSVKDRLTISRDDSQSMLYLQMNNLKTEDTAMYYCVRWFRAMDYWGQGTSVTVSS

SEQ ID NO: 272 - protein, 118 aa, synthetic construct
EVQLVESGGGLVKPGGSLRLSCAASGFTFSMYGMHWVRQAPGKGLEWVGHIRSKSSNYATYYADSVKDRFTISRDDSKNTLYLQMNSLKTEDTAVYYCTTWFRAMDYWGQGTLVTVSS

SEQ ID NO: 273 - protein, 118 aa, synthetic construct; identical to SEQ ID NO: 272

SEQ ID NO: 274 - protein, 118 aa, synthetic construct
EVQLVESGGGLVQPGGSLRLSCAASGFTFSMYGMHWVRQAPGKGLEWVSHIRSKSSNYATYYADSVKDRFTISRDNAKNSLYLQMNSLRAEDTAVYYCARWFRAMDYWGQGTLVTVSS

SEQ ID NO: 275 - protein, 118 aa, synthetic construct
EVQLVESGGGLVQPGGSLKLSCAASGFTFSMYGMHWVRQASGKGLEWVGHIRSKSSNYATYYADSVKDRFTISRDDSKNTAYLQMNSLKTEDTAVYYCTRWFRAMDYWGQGTLVTVSS

SEQ ID NO: 276 - protein, 118 aa, synthetic construct
EVQLVESGGGLVQPGGSLRLSCAASGFTFSMYGMHWVRQAPGKGLEWVGHIRSKSSNYATYYADSVKDRFTISRDDSKNSLYLQMNSLKTEDTAVYYCARWFRAMDYWGQGTLVTVSS

SEQ ID NO: 277 - protein, 118 aa, synthetic construct; identical to SEQ ID NO: 274

SEQ ID NO: 278 - protein, 118 aa, synthetic construct
EVQLVESGGGLEQPGGSLRLSCAGSGFTFRMYGMHWVRQAPGKGLEWVSHIRSKSSNYATYYADSVKDRFTISRDNSKNTLYLQMNSLRAEDTAVYYCAKWFRAMDYWGQGTTVTVSS

SEQ ID NO: 279 - protein, 118 aa, synthetic construct
EVQLVESGGGLVQPGGSLKLSCAASGFTFSMYGMHWVRQASGKGLEWVGHIRSKSSNYATYYADSVKDRFTISRDDSKNTAYLQMNSLKTEDTAVYYCTRWFRAMDYWGQGTTVTVSS

SEQ ID NO: 280 - protein, 107 aa, synthetic construct
DIVMTQSHKFMSTSVGDRVSITCKASQDVSTAVAWYQEKPGQSPKLLIYWASTRHTGVPDRFTGSGSGTDYTLTISSVQAEDLALYYCQQHYSTPLTFGAGTKLELK

SEQ ID NO: 281 - protein, 111 aa, synthetic construct
DIVVTQSPASLAVSLGQRATISCRASKSVSTSGYSYMHWYQQKPGQPPKLLIYLASNLESGVPARFSGSGSGTDFTLNIHPVEEEDVAIYYCQHSRELPLTFGAGTKLELK

SEQ ID NO: 282 - protein, 107 aa, synthetic construct
DIQMTQSPSSLSASLGERVSLTCRASQEISGYLSWLQQKPDGTIKRLIYAASTLDSGVPKRFSGSRSGSDYSLTISSLESEDFADYYCLQYGSYPYTFGGGTKLEIK

SEQ ID NO: 283 - protein, 111 aa, synthetic construct
DIVLTQSPASLTVSLGQRATISCRASKSVSTSGYSYMHWYQQKPGQPPKLLIYLASNLESGVPARFSGSGSGTDFTLNIHPVEEEDAATYYCQHSRELPITFGSGTKLEIK

SEQ ID NO: 284 - protein, 111 aa, synthetic construct
DIVLTQSPASLVVSLGQRATISCRASQSVSTSGYSYMHWYQQKPGQPPKLLIYLASNVQSGVPARFSGSGSGTDFTLNIHPVEEEDVATYYCHHNRDLPFTFGSGTKLEIK

SEQ ID NO: 285 - protein, 111 aa, synthetic construct; identical to SEQ ID NO: 281

SEQ ID NO: 286 - protein, 111 aa, synthetic construct
DIVLTQSPASLAVSLGQRATISCRASKSVSASGYSYMHWYQQKPGQPPKLLIYLASNLQSGVPARFSGSGSGTDFTLNIHPVEEEDAATYYCQHSRELPPTFGGGTKLEIK

SEQ ID NO: 287 - protein, 111 aa, synthetic construct
DIVLTQSPASLAVFLGQRATISCRASKSVSTSGYSYMHWYQQKAGQPPKLLIYLASNLESGVPARFSGSGSGTDFTLNIHPVEEEDAATYYCHHSRELPITFGSGTKLEMK

SEQ ID NO: 288 - protein, 107 aa, synthetic construct; identical to SEQ ID NO: 280

SEQ ID NO: 289 - protein, 107 aa, synthetic construct
EIVLTQSPGTLSLSPGERATLSCKASQDVSTAVAWYQQKPGQAPRLLIYWASTRHTGIPDRFSGSGSGTDFTLTISRLEPEDFAVYYCQQHYSTPLTFGQGTKVEIK

SEQ ID NO: 290 - protein, 107 aa, synthetic construct
DIVMTQSPLSLPVTPGEPASISCKASQDVSTAVAWYLQKSGQSPQLLIYWASTRHTGVPDRFSGSGSGTDFTLKISRVEAEDVGFYYCQQHYSTPLTFGQGTKLEIK

SEQ ID NO: 291 - protein, 107 aa, synthetic construct
DIVMTQSPDSLAVSLGERATINCKASQDVSTAVAWYQQKPGQPPKLLIYWASTRHTGVPDRFSGSGSGTDFTLTISSLQAEDVAVYYCQQHYSTPLTFGGGTKVEIK

SEQ ID NO: 292 - protein, 107 aa, synthetic construct
DIQMTQSPSSLSASVGDRVTITCKASQDVSTAVAWYQQKPGKVPKLLIYWASTRHTGVPSRFSGSGSGTDFTLTISSLQPEDVATYYCQQHYSTPLTFGGGTKVEIK

SEQ ID NO: 293 - protein, 107 aa, synthetic construct
DIQLTQSPSFLSASVGDRVTITCKASQDVSTAVAWYQQKPGKAPKLLIYWASTRHTGVPSRFSGSGSGTEFTLTISSLQPEDFATYYCQQHYSTPLTFGGGTKVEIK

SEQ ID NO: 294 - protein, 107 aa, synthetic construct
DIQMTQSPSSLSASVGDRVTITCKASQDVSTAVAWYQQKPGKAPKLLLYWASTRHTGVPSRFSGSGSGTDYTLTISSLQPEDFATYYCQQHYSTPLTFGGGTKVEIK

SEQ ID NO: 295 - protein, 107 aa, synthetic construct
AIRMTQSPFSLSASVGDRVTITCKASQDVSTAVAWYQQKPAKAPKLFIYWASTRHTGVPSRFSGSGSGTDYTLTISSLQPEDFATYYCQQHYSTPLTFGGGTKVEIK